(19) United States
(12) Patent Application Publication
(10) Pub. No.: US 2025/0032591 A1
(43) Pub. Date: Jan. 30, 2025

(54) PHARMACEUTICAL COMPOSITIONS AND METHODS FOR TREATING CONNECTIVE TISSUE CONDITIONS WITH COLLAGEN POLYPEPTIDES

(71) Applicant: Geltor, Inc., Emeryville, CA (US)

(72) Inventors: Nikolay OUZOUNOV, Lafayette, CA (US); Jeffrey R. MELLIN, Richmond, CA (US); Julia CO, Oakland, CA (US)

(21) Appl. No.: 18/420,597

(22) Filed: Jan. 23, 2024

Related U.S. Application Data
(63) Continuation of application No. PCT/US2022/038568, filed on Jul. 27, 2022.
(60) Provisional application No. 63/226,591, filed on Jul. 28, 2021.

(51) Int. Cl. A61K 38/39; C07K 14/78

(52) U.S. Cl. CPC A61K 38/39; C07K 14/78

(57) ABSTRACT

Provided herein are therapeutic compositions comprising non-naturally occurring polypeptides for the treatment of skin conditions.

### CROSS-REFERENCE

This application is a continuation of International Application No. PCT/US2022/038568, filed Jul. 27, 2022, which claims the benefit of U.S. Provisional Application No. 63/226,591, filed Jul. 28, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 19, 2024, is named 57607_719_301_SL.xml and is 116,127 bytes in size.

### BACKGROUND

Collagen is one of the most abundant proteins found in various connective tissues in the body including tendons, ligaments, skin, and hair. Collagens or collagen supplements are popular in medical, cosmetic, and/or health purposes (e.g., stimulating skin growth, promoting wound healing, strengthening nails or joints, etc.). Collagens for most collagen supplements are derived from animals as a byproduct of the animal processing industry. Yet, such animal-derived collagens may increase the risk of illness transmission as well as allergies. Moreover, certain consumers are generally interested in animal-free products for a variety of other reasons. Thus, there remains a need for improved compositions and methods of collagens derived from non-animal sources.

### SUMMARY

In one aspect, a method of treating a wound in a subject is provided, the method comprising: administering to the subject a therapeutically effective amount of a polypeptide comprising or consisting of an amino acid sequence having at least 80% sequence identity to SEQ ID NO: 32, or a polypeptide comprising or consisting of an amino acid sequence having at least 80% sequence identity to a truncate of SEQ ID NO: 32, thereby treating the wound. In some embodiments, the wound exhibits impaired wound healing. In some embodiments, the administering comprises administering the polypeptide to the wound or to skin adjacent to the wound.

In another aspect, a method of treating a proliferative disorder of the skin is provided, the method comprising: administering to the subject a therapeutically effective amount of a polypeptide comprising or consisting of an amino acid sequence having at least 80% sequence identity to SEQ ID NO: 32, or a polypeptide comprising or consisting of an amino acid sequence having at least 80% sequence identity to a truncate of SEQ ID NO: 32, thereby treating the proliferative disorder of the skin, wherein the proliferative disorder of the skin is characterized by abnormal proliferation, migration, and/or adhesion of skin cells (e.g., fibroblasts, keratinocytes). In some embodiments, the abnormal proliferation, migration, and/or adhesion is decreased or reduced proliferation, migration, and/or adhesion. In some embodiments, the skin condition is epidermal thinning, epidermal atrophy, dermal atrophy, epidermal degeneration, acantholysis, pemphigus foliaceus, pemphigus vulgaris, acantholytic dyskeratosis, Darier disease, Hailey-Hailey disease, Grover disease, lichen sclerosus, hyalinisation of collagen, or a combination thereof.

In any of the preceding embodiments, the polypeptide comprises or consists of an amino acid sequence having at least 85% sequence identity to SEQ ID NO: 32, or comprises or consists of an amino acid sequence having at least 85% sequence identity to a truncate of SEQ ID NO: 32. In any of the preceding embodiments, the polypeptide comprises or consists of an amino acid sequence having at least 90% sequence identity to SEQ ID NO: 32, or comprises or consists of an amino acid sequence having at least 90% sequence identity to a truncate of SEQ ID NO: 32. In any of the preceding embodiments, the polypeptide comprises or consists of an amino acid sequence having at least 95% sequence identity to SEQ ID NO: 32, or comprises or consists of an amino acid sequence having at least 95% sequence identity to a truncate of SEQ ID NO: 32. In any of the preceding embodiments, the polypeptide comprises or consists of an amino acid sequence having at least 98% sequence identity to SEQ ID NO: 32, or comprises or consists of an amino acid sequence having at least 98% sequence identity to a truncate of SEQ ID NO: 32. In any of the preceding embodiments, the polypeptide comprises or consists of an amino acid sequence having 100% sequence identity to SEQ ID NO: 32, or comprises or consists of an amino acid sequence having 100% sequence identity to a truncate of SEQ ID NO: 32. In any of the preceding embodiments, the truncate of SEQ ID NO: 32 comprises an N-terminal truncation, a C-terminal truncation, or both, relative to SEQ ID NO: 32. In any of the preceding embodiments, the N-terminal truncation is an N-terminal truncation of 50 amino acids to 750 amino acids relative to SEQ ID NO: 32. In any of the preceding embodiments, the C-terminal truncation is a C-terminal truncation of 50 amino acids to 600 amino acids relative to SEQ ID NO: 32. In any of the preceding embodiments, the polypeptide comprises or consists of the amino acid sequence of SEQ ID NO: 8. In any of the preceding embodiments, the polypeptide has a total truncation of 50 amino acids to 1250 amino acids. In any of the preceding embodiments, the polypeptide is at least 50 amino acids in length. In any of the preceding embodiments, the polypeptide is 50 amino acids to 250 amino acids in length. In any of the preceding embodiments, the polypeptide does not comprise one or more of: a laminin G domain, a Von Willebrand factor type A (vWA) domain, and a fibrillar collagen C-terminal domain. In any of the preceding embodiments, the polypeptide comprises one or more collagen triple helix repeats. In any of the preceding embodiments, the polypeptide is monomeric. In any of the preceding embodiments, the polypeptide does not form a stable triple helix structure of a naturally occurring collagen. In any of the preceding embodiments, the polypeptide is substantially free of other collagen chains. In any of the preceding embodiments, the polypeptide has a non-naturally occurring level of hydroxylation relative to a naturally-occurring collagen. In any of the preceding embodiments, fewer than 10% of prolines present in the polypeptide are hydroxylated. In any of the preceding embodiments, the polypeptide is non-hydroxylated. In any of the preceding embodiments, the polypeptide has a non-naturally occurring level of glycosylation relative to a naturally-occurring collagen. In any of the preceding embodiments, the polypeptide comprises less than 5 wt. % glycosylation. In any of the preceding embodiments, the polypeptide is administered as a pharmaceutical composition. In any of the preceding embodiments, the pharmaceutical composition further comprises a pharmaceutically acceptable excipient. In any of the preceding embodiments, the pharmaceutically acceptable excipient is selected from the group consisting of: an antiadherent, a binder, a coating, a color, a disintegrant, a flavor, a glidant, a lubricant, a preservative, a sorbent, a vehicle, and any combination thereof. In any of the preceding embodiments, the pharmaceutical composition is formulated for topical administration. In any of the preceding embodiments, the pharmaceutical composition is formulated as a gel, a cream, a lotion, an oil, a foam, an ointment, a serum, and any combination thereof. In any of the preceding embodiments, after the administering, keratinocyte growth and/or regeneration in the skin is increased (e.g., relative to prior to the administering) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In any of the preceding embodiments, after the administering, collagen production in the skin is increased (e.g., relative to prior to the administering) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In any of the preceding embodiments, after the administering, fibroblast migration, proliferation, and/or adhesion in the skin is increased (e.g., relative to prior to the administering) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In any of the preceding embodiments, after the administering, keratinocyte viability after exposure to urban dust is increased (e.g., relative to prior to the applying) by at least about 5%, at least about 10%, at least about 15%, at least about 20%, at least about 25%, at least about 30%, at least about 35%, at least about 40%, at least about 45%, at least about 50%, at least about 55%, at least about 60%, at least about, at least about 65%, at least about 70%, or at least about 75%. In any of the preceding embodiments, after the administering, expression (e.g., by keratinocytes, fibroblasts) of one or more genes involved in a signaling pathway selected from the group consisting of: VEGFA/VEGFR2 signaling pathway, focal adhesion signaling pathway, endothelin signaling pathway, EGF/EGFR signaling pathway, TGF-beta signaling pathway, and any combination thereof, is increased. In any of the preceding embodiments, the one or more genes involved in VEGFA/VEGFR2 signaling pathway is selected from the group consisting of: MYOC1, FLII, ROCK1, ROCK2, CLTC, LIMK 1, EGR1, and any combination thereof. In any of the preceding embodiments, the one or more genes involved in focal adhesion signaling pathway is selected from the group consisting of: ITGA3, TNC, LAMC1, FLNA, TLN1, ZYX, DIAPH1, and any combination thereof. In any of the preceding embodiments, the one or more genes involved in endothelin signaling pathway is selected from the group consisting of: TRIOBP, WNK1, MMP2, VCAN, ACTA2, GNA12, EGR1, and any combination thereof. In any of the preceding embodiments, the one or more genes involved in EGF/EGFR signaling pathway is selected from the group consisting of: ATXN2, JAK1, RPS6KA2, ROCK1, SHC1, IQGAP1, PLCG1, and any combination thereof. In any of the preceding embodiments, the one or more genes involved in TGF-beta signaling pathway is selected from the group consisting of: SMURF1, SPTBN1, PAK2, ROCK1, SHC1, TGFBR3, TGFBR1, and any combination thereof.

In another aspect, a pharmaceutical composition is provided comprising a polypeptide comprising or consisting of an amino acid sequence having at least 80% sequence identity to SEQ ID NO: 32, or a polypeptide comprising or consisting of an amino acid sequence having at least 80% sequence identity to a truncate of SEQ ID NO: 32; and a pharmaceutically acceptable excipient. In some embodiments, the polypeptide comprises or consists of an amino acid sequence having at least 85% sequence identity to SEQ ID NO: 32, or comprises or consists of an amino acid sequence having at least 85% sequence identity to a truncate of SEQ ID NO: 32. In some embodiments, the polypeptide comprises or consists of an amino acid sequence having at least 90% sequence identity to SEQ ID NO: 32, or comprises or consists of an amino acid sequence having at least 90% sequence identity to a truncate of SEQ ID NO: 32. In some embodiments, the polypeptide comprises or consists of an amino acid sequence having at least 95% sequence identity to SEQ ID NO: 32, or comprises or consists of an amino acid sequence having at least 95% sequence identity to a truncate of SEQ ID NO: 32. In some embodiments, the polypeptide comprises or consists of an amino acid sequence having at least 98% sequence identity to SEQ ID NO: 32, or comprises or consists of an amino acid sequence having at least 98% sequence identity to a truncate of SEQ ID NO: 32. In some embodiments, the polypeptide comprises or consists of an amino acid sequence having 100% sequence identity to SEQ ID NO: 32, or comprises or consists of an amino acid sequence having 100% sequence identity to a truncate of SEQ ID NO: 32. In some embodiments, the truncate of SEQ ID NO: 32 comprises an N-terminal truncation, a C-terminal truncation, or both, relative to SEQ ID NO: 32. In some embodiments, the N-terminal truncation is an N-terminal truncation of 50 amino acids to 750 amino acids relative to SEQ ID NO: 32. In some embodiments, the C-terminal truncation is a C-terminal truncation of 50 amino acids to 600 amino acids relative to SEQ ID NO: 32. In some embodiments, the polypeptide comprises or consists of the amino acid sequence of SEQ ID NO: 8. In some embodiments, the polypeptide has a total truncation of 50 amino acids to 1250 amino acids. In some embodiments, the polypeptide is at least 50 amino acids in length. In some embodiments, the polypeptide is 50 amino acids to 250 amino acids in length. In some embodiments, the polypeptide does not comprise one or more of: a laminin G domain, a Von Willebrand factor type A (vWA) domain, and a fibrillar collagen C-terminal domain. In some embodiments, the polypeptide comprises one or more collagen triple helix repeats. In some embodiments, the polypeptide is monomeric. In some embodiments, the polypeptide does not form a stable triple helix structure of a naturally occurring collagen. In some embodiments, the polypeptide is substantially free of other collagen chains. In some embodiments, the polypeptide has a non-naturally occurring level of hydroxylation relative to a naturally-occurring collagen. In some embodiments, fewer than 10% of prolines present in the polypeptide are hydroxylated. In some embodiments, the polypeptide is non-hydroxylated. In some embodiments, the polypeptide has a non-naturally occurring level of glycosylation relative to a naturally-occurring collagen. In some embodiments, the polypeptide comprises less than 5 wt. % glycosylation. In some embodiments, the pharmaceutically acceptable excipient is selected from the group consisting of: an antiadherent, a binder, a coating, a color, a disintegrant, a flavor, a glidant, a lubricant, a preservative, a sorbent, a vehicle, and any combination thereof. In some embodiments, the pharmaceutical composition is formulated for topical administration. In some embodiments, the pharmaceutical composition is formulated as a gel, a cream, a lotion, an oil, a foam, an ointment, a serum, and any combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1 depicts alignment of non-naturally occurring polypeptides of the disclosure with corresponding naturally occurring collagens. FIG. 1 discloses SEQ ID NO: 33 (a subsection of SEQ ID NO: 31).

FIG. 2 depicts alignment of non-naturally occurring polypeptides of the disclosure with corresponding naturally occurring collagens. FIG. 2 discloses SEQ ID NO: 34 (a subsection of SEQ ID NO: 32).

FIG. 3 depicts an image of two SDS-PAGE gels showing bands of collagen proteins in supernatant samples from microbial cell cultures. The identities of each protein are indicated above each band.

FIGS. 4A-4C depict images of SDS-PAGE gels showing bands of non-naturally occurring polypeptides of the disclosure before and after pH 3.0 treatment.

FIGS. 5A-5C depict viability of an immortalized human keratinocyte cell line, human primary fibroblasts, and human primary keratinocytes after exposure to an exemplary non-naturally occurring polypeptide of the disclosure.

FIG. 6 depicts a dose-dependent increase in proliferation of human primary keratinocytes after exposure to an exemplary non-naturally occurring polypeptide of the disclosure.

FIG. 7 depicts a dose-dependent increase in collagen I production by primary human fibroblasts after exposure to an exemplary non-naturally occurring polypeptide of the disclosure.

FIG. 8 depicts wound healing activity of human dermal fibroblasts after exposure to an exemplary non-naturally occurring polypeptide of the disclosure.

### DETAILED DESCRIPTION

The terminology used herein is for the purpose of describing particular cases only and is not intended to be limiting. As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The terms “about” or “approximately” mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual”, “patient”, or “subject” are used interchangeably herein. None of the terms require or are limited to a situation characterized by the supervision (e.g., constant or intermittent) of a health care worker (e.g., a doctor, a registered nurse, a nurse practitioner, a physician's assistant, an orderly, or a hospice worker).

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of”. The phrase “consisting essentially of” is used herein to require the specified feature(s) as well as those which do not materially affect the character or function of the claimed disclosure. As used herein, the term “consisting” is used to indicate the presence of the recited feature alone.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as any individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terms “treatment” or “treating” are used herein interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject is still afflicted with the underlying disorder. For prophylactic benefit, the compositions are, in some embodiments, administered to a subject at risk of developing a particular disease or condition, or to a subject reporting one or more of the physiological symptoms of a disease, even though a diagnosis of this disease has not been made.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “pharmaceutically acceptable excipient” or “pharmaceutically acceptable carrier” are used interchangeably herein and refer to a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “truncated collagen” as used herein generally refers to a polypeptide that is smaller than a full-length (e.g., natural) collagen wherein one or more portions of the full-length (e.g., natural) collagen is not present. The non-naturally occurring polypeptides provided herein may be truncated at the C-terminal end, the N-terminal end, truncated by removal of internal portion(s) of the full-length collagen sequence (e.g., an internal truncation), truncated at both the C-terminal end and the N-terminal end, or may have one or both of a C-terminal truncation and an N-terminal truncation as well as an internal truncation. In a non-limiting embodiment, a truncated collagen may comprise an amino acid sequence according to SEQ ID NO: 2, or a homolog thereof. In another non-limiting embodiment, a truncated collagen may comprise an amino acid sequence according to SEQ ID NO: 8, or a homolog thereof.

When used in reference to an amino acid position, a “truncation” is inclusive of said amino acid position. For example, an N-terminal truncation at amino acid position 100 relative to a full-length polypeptide means a truncation of 100 amino acids from the N-terminus of the full-length polypeptide (i.e., the truncated polypeptide is missing amino acid positions 1 through 100 of the full-length polypeptide). Similarly, a C-terminal truncation at amino acid position 901 of a full-length polypeptide (assuming a 1000 amino acid full-length polypeptide) means a truncation of 100 amino acids from the C-terminus (i.e., the truncated polypeptide is missing amino acid positions 901 through 1000 of the full-length polypeptide). Similarly, an internal truncation at amino acid positions 101 and 200 means an internal truncation of 100 amino acids of the full-length polypeptide (i.e., the truncated polypeptide is missing amino acid positions 101 to 200 of the full-length polypeptide).

The section headings used herein are for organizational purposes only and are not to be construed as limiting the subject matter described.

Provided herein, in certain embodiments, are compositions (e.g., pharmaceutical compositions) and methods for treating various skin disorders, diseases, and/or conditions. The skin disorders, diseases, and/or conditions may be, in some cases, characterized by abnormal or impaired wound healing. The skin disorders, diseases, and/or conditions may be, in some cases, characterized by impaired keratinocyte and/or fibroblast proliferation, migration, and/or adhesion. The compositions (e.g., pharmaceutical compositions) and methods provided herein generally involve the use of any polypeptide provided herein. In a non-limiting example, the polypeptide comprises or consists of an amino acid sequence having at least 80% sequence identity to SEQ ID NO: 32, or comprises or consists of an amino acid sequence having at least 80% sequence identity to a truncate of SEQ ID NO: 32 (e.g., as described herein).

Further provided in certain embodiments herein are, by way of non-limiting example, compositions, methods, and systems for manufacturing non-naturally occurring polypeptides, such as, e.g., animal-free collagen polypeptides or collagen-like polypeptides, as well as collagen fragments, and/or truncated collagens, such as that are expressed in and/or by genetically engineered microorganisms. Thus, in various aspects of the disclosure, the non-naturally occurring polypeptides provided herein include collagen or collagen-like polypeptides, recombinant collagens, collagen fragments, or truncated collagens. In certain embodiments, the non-naturally occurring polypeptides described herein (e.g., recombinant collagens, collagen fragments, or truncated collagens) are derived from any suitable source, such as from mammalian or non-mammalian sources. For example, in some embodiments, the non-naturally occurring polypeptides described herein (e.g., recombinant collagens, collagen fragments, or truncated collagens), or at least a portion thereof, are derived from (e.g., modified, truncated, fragments of, or the like) collagens of a bird or an avian animal (e.g., Gallus gallus collagen), a freshwater- or saltwater-fish (e.g., Acipenser schrenckii collagen), or any combination thereof.

The non-naturally occurring polypeptides provided herein are not normally found in nature. Generally, the non-naturally occurring polypeptides described herein exhibit one or more differences from naturally occurring collagens. In certain aspects, the non-naturally occurring polypeptides provided herein may have a different amino acid sequence from naturally occurring polypeptides (e.g., a truncated collagen). In some cases, the non-naturally occurring polypeptides may have a different structure from a naturally occurring collagen. The quaternary structure of natural collagen is a triple helix, typically composed of three polypeptides. In some aspects, the non-naturally occurring polypeptides described herein may not have or may not form a quaternary structure of natural collagen. For example, in some instances, the non-naturally occurring polypeptides described herein may not form the stable triple helical structure of naturally occurring collagen. In certain instances, of the three polypeptides that form natural collagen, two are usually identical and are designated as the alpha chain. The third polypeptide is designated as the beta chain. In certain instances, a typical natural collagen can be designated as AAB, wherein the collagen is composed of two alpha (“A”) strands and one beta (“B”) strand. In some aspects, the non-naturally occurring polypeptides described herein do not have the AAB structure of natural collagen. In some instances, the non-naturally occurring polypeptides described herein are free from or substantially free from different collagen chains (e.g., a non-naturally occurring polypeptide described herein may comprise an alpha chain collagen and may be free or substantially free from a beta chain collagen). In some aspects, the non-naturally occurring polypeptides described herein are monomeric and/or do not form multimeric structures. In other aspects, the non-naturally occurring polypeptides described herein may, in some instances, form multimeric structures with identical monomers (e.g., homodimers, homotrimers, etc.).

In some aspects, the non-naturally occurring polypeptides are recombinant polypeptides (e.g., prepared recombinantly in a host cell). The non-naturally occurring polypeptide is, in one embodiment, a truncated collagen. Other non-naturally occurring collagen polypeptides include chimeric collagens. A chimeric collagen is a polypeptide wherein one portion of a collagen polypeptide is contiguous with a portion of a second collagen polypeptide. For example, a collagen molecule comprising a portion of a collagen from one species contiguous with a portion of a collagen from another species is a chimeric collagen. In another embodiment, the non-naturally occurring polypeptide comprises a fusion polypeptide that includes additional amino acids such as a secretion tag, histidine tag, green fluorescent protein, protease cleavage site, GEK repeats, GDK repeats, and/or beta-lactamase.

In some embodiments, the non-naturally occurring polypeptides (e.g., recombinant polypeptides) provided herein have a non-naturally occurring level of glycosylation, for example, relative to a corresponding natural collagen or naturally present collagen. For example, in some embodiments, the non-naturally occurring polypeptide (e.g., recombinant polypeptide) comprises less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, less than about 1 wt. %, less than about 0.9 wt. %, less than about 0.8 wt. %, less than about 0.7 wt. %, less than about 0.6 wt. %, less than about 0.5 wt. %, less than about 0.4 wt. %, less than about 0.3 wt. %, less than about 0.2 wt. %, or less than about 0.1 wt. % glycosylation. Alternatively and/or additionally, the non-naturally occurring polypeptide (e.g., recombinant polypeptide) comprises less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% of total glycosylation of the corresponding natural collagen or naturally present collagen. For example, where the naturally present collagen ABC from a species XYZ has 20 glycosylations (throughout the full length of the collagen ABC or a portion thereof), it is contemplated that the non-naturally occurring polypeptide (e.g., recombinant polypeptide) comprises less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, or less than 1 glycosylations. In some embodiments, those lower levels of glycosylation can be specific to one or more types of glycosylation (e.g., O-glycosylation or N-glycosylation, etc.) and/or the glycosylation residues (e.g., galactosylhydroxylysine (Gal-Hyl), glucosyl galactosylhydroxylsine (GlcGal-Hyl), etc.). Non-naturally occurring polypeptides produced recombinantly (e.g., in a recombinant host cell), in some instances, may have a glycosylation level and/or a glycosylation pattern that differs from naturally occurring collagen.

In some aspects, a non-naturally occurring polypeptide provided herein has a non-naturally occurring amount of hydroxyprolines. In some cases, a non-naturally occurring polypeptide provided herein lacks hydroxyprolines. In some cases, a non-naturally occurring polypeptide provided herein comprises fewer hydroxyprolines than a naturally-occurring collagen. Hydroxyprolines include, without limitation, 3-hydroxyproline, 4-hydroxyproline, and 5-hydroxyproline. In some cases, less than about 50% (e.g., less than about 45%, less than about 40%, less than about 35%, less than about 30%, less than about 25%, less than about 20%, less than about 15%, less than about 10%, or less) of the prolines present in the amino acid sequence of a non-naturally occurring polypeptide provided herein are hydroxyprolines. In some aspects, a non-naturally occurring polypeptide produced recombinantly (e.g., in a recombinant host cell) may have fewer hydroxyprolines than a naturally occurring collagen. In some cases, a recombinant polypeptide as provided herein is recombinantly expressed in a recombinant host cell (e.g., bacterial cell, yeast cell, fungal cell) that lacks an enzyme that hydroxylates one or more amino acids (e.g., proline) of the recombinant polypeptide. In some cases, a recombinant polypeptide as provided herein is recombinantly expressed in a host cell (e.g., bacterial cell, yeast cell, fungal cell) that lacks prolyl 4-hydroxylase and/or prolyl 3-hydroxylase.

In some aspects, the non-naturally occurring polypeptides provided herein lack or substantially lack lysyl oxidation. Lysyl oxidation involves the conversion of lysine residues into highly reactive aldehydes that can form cross-links with other proteins. Naturally occurring collagens may have some level of lysyl oxidation. Thus, the non-naturally occurring polypeptides may be different from natural collagens in that they lack or substantially lack lysyl oxidation. In some cases, less than about 50% (e.g., less than about 45%, less than about 40%, less than about 35%, less than about 30%, less than about 25%, less than about 20%, less than about 15%, less than about 10%, or less) of the lysines present in the amino acid sequence of a non-naturally occurring polypeptide provided herein are oxidized.

Generally, the non-naturally occurring polypeptides provided herein (e.g., truncated collagens) may have a function and/or provide a benefit (e.g., as provided herein) similar or substantially similar to that of a natural or a full-length collagen. In some cases, the non-naturally occurring polypeptides provided herein (e.g., truncated collagens) may have improved or increased function and/or benefit (e.g., as provided herein) as compared to a natural or a full-length collagen. In some embodiments, the non-naturally occurring polypeptides provided herein may have one or more different functions as compared to a natural or a full-length collagen.

The non-naturally occurring polypeptides disclosed herein often have advantageous properties related to their monomeric structure and/or lack of amino acids capable of cross-linking with other collagen strands, e.g., the lack of hydroxyproline residues. In addition, collagen hydrolysates of the non-naturally occurring polypeptides disclosed herein are also produced with increased solubility as compared to full-length or natural collagens. Moreover, monomeric structures, as opposed to natural triple helix collagens, are more readily digestible and bioavailable, or broken down by digestive proteases. Other advantageous properties include improved physical properties in liquid compositions and in purification processes, since full-length or natural collagens or collagen strands interact to form stronger structures that can precipitate due to the presence of hydroxyproline residues.

In certain preferred embodiments, the non-naturally occurring polypeptides provided herein (e.g., truncated collagens) comprise an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to at least a portion of the naturally existing mammalian or non-mammalian collagens from which those are derived from. In some instances, a portion or portions of a natural amino acid sequence is deleted, but the remainder of the sequence is substantially similar or identical to the natural amino acid sequence. In certain exemplary embodiments, the non-naturally occurring polypeptide has an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to a Gallus gallus Type 21 alpha 1 collagen or a truncate or a fragment thereof. In another example, the non-naturally occurring polypeptide has an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to a Acipenser schrenckii Type 2 alpha 1 collagen or a truncate or a fragment thereof.

In some embodiments, the recombinant polypeptide is a truncated collagen. In certain instances, a truncated collagen is a polypeptide that is smaller than a full-length (e.g., natural) collagen wherein one or more portions (e.g., internal and/or terminal portion(s)) of the full-length (e.g., natural) collagen is not present. In various instances, the non-naturally occurring polypeptides provided herein (e.g., truncated collagens) are truncated at the C-terminal end, the N-terminal end, truncated by removal of internal portion(s) of the full-length collagen polypeptide (e.g., internal truncation), truncated at both the C-terminal end and the N-terminal end, or comprise one or both of a C-terminal truncation and an N-terminal truncation as well as an internal truncation. In some instances, the non-naturally occurring polypeptide is a fragment of a naturally occurring collagen that retains at least about 50%, at least about 60%, at least about 70%, at least about 80%, or at least about 90% of a function (e.g., of interest) of natural or naturally-present corresponding collagens. In some instances, the term truncated collagen is interchangeably used with the term collagen fragment. In some instances, the truncated collagen includes any contiguous collagen fragments that are at least about 10%, at least about 20%, at least about 30%, at least about 40%, at least about 50%, at least about 60%, at least about 70%, or at least about 80% of full-length natural or naturally-present corresponding collagens. In some embodiments, the truncation is an internal truncation, a truncation at the N-terminal portion of the collagen, a truncation at the C-terminal portion of the collagen, a truncation of an internal portion, or a truncation at both the C-terminal end and the N-terminal end. A truncated collagen provided herein may be truncated by 50 amino acids to 1250 amino acids, 50 amino acids to 1200 amino acids, 50 amino acids to 1150 amino acids, 50 amino acids to 1100 amino acids, 50 amino acids to 1050 amino acids, 50 amino acids to 1000 amino acids, 50 amino acids to 950 amino acids, 50 amino acids to 900 amino acids, 50 amino acids to 850 amino acids, 50 amino acids to 800 amino acids, 50 amino acids to 750 amino acids, 50 amino acids to 700 amino acids, 50 amino acids to 650 amino acids, 50 amino acids to 600 amino acids, 50 amino acids to 550 amino acids, 50 amino acids to 500 amino acids, 50 amino acids to 450 amino acids, 50 amino acids to 400 amino acids, 50 amino acids to 350 amino acids, 50 amino acids to 300 amino acids, 50 amino acids to 250 amino acids, 50 amino acids to 200 amino acids, 50 amino acids to 150 amino acids, or 50 amino acids to 100 amino acids (e.g., relative to a full-length collagen). In another embodiment, a truncated collagen is truncated by 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900, 950, 1000, 1050, 1100, 1150, 1200, or 1250 amino acids (e.g., relative to a full-length collagen).

In some embodiments, a polypeptide provided herein (e.g., amino acid sequence thereof) may be truncated at the C-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein (e.g., amino acid sequence thereof) may be truncated at the C-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

In some embodiments, a polypeptide provided herein (e.g., amino acid sequence thereof) may be truncated at the N-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein may be truncated at the N-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

In some embodiments, a polypeptide provided herein (e.g., amino acid sequence thereof) may be truncated at both the N-terminal end and the C-terminal end relative to a full-length collagen. In some instances, a polypeptide provided herein may be truncated at the N-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like; and may be truncated at the C-terminal end (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein may be truncated at the N-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids; and may be truncated at the C-terminal end (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

In some embodiments, a polypeptide provided herein (e.g., amino acid sequence thereof) may be internally truncated (relative to a full-length collagen) by any suitable number of amino acid residues, such as up to 10, 10 to 800, 10 to 700, 10 to 600, 10 to 500, 10 to 400, 10 to 300, 10 to 200, 10 to 100, 50 to 800, 50 to 700, 50 to 600, 50 to 500, 50 to 400, 50 to 300, 50 to 200, 50 to 100, or the like. In some cases, a polypeptide provided herein may be internally truncated (relative to a full-length collagen) by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more amino acids.

A non-naturally occurring polypeptide (e.g., truncated collagen) disclosed herein may comprise a truncation relative to a full-length (e.g., natural) collagen. In some embodiments, a truncated collagen disclosed herein may comprise a truncation relative to a full-length (e.g., natural) chicken (Gallus gallus) type 21 alpha 1 collagen (e.g., SEQ ID NO: 31). In some embodiments, a truncated collagen disclosed herein may comprise the amino acid sequence of SEQ ID NO: 31, or an amino acid sequence having at least about 70% sequence identity (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater) to the amino acid sequence of SEQ ID NO: 31, with an N-terminal truncation, a C-terminal truncation, an internal truncation, or a combination thereof. In some embodiments, a truncated collagen disclosed herein may comprise a truncation relative to a full-length (e.g., natural) Japanese sturgeon (Acipenser schrenckii) type 2 alpha 1 collagen (e.g., SEQ ID NO: 32). In some embodiments, a truncated collagen disclosed herein may comprise the amino acid sequence of SEQ ID NO: 32, or an amino acid sequence having at least about 70%-sequence identity (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater) to the amino acid sequence of SEQ ID NO: 32, with an N-terminal truncation, a C-terminal truncation, an internal truncation, or a combination thereof. In some embodiments, a truncated collagen disclosed herein may comprise a truncation relative to a full-length (e.g., natural) jellyfish (Hydrozoan) collagen (e.g., SEQ ID NO: 39). In some embodiments, a truncated collagen disclosed herein may comprise the amino acid sequence of SEQ ID NO: 39, or an amino acid sequence having at least about 70% sequence identity (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater) to the amino acid sequence of SEQ ID NO: 39, with an N-terminal truncation, a C-terminal truncation, an internal truncation, or a combination thereof. In some embodiments, a truncated collagen disclosed herein may comprise a truncation relative to a full-length (e.g., natural) human type 21 alpha 1 collagen (e.g., SEQ ID NO: 40). In some embodiments, a truncated collagen disclosed herein may comprise the amino acid sequence of SEQ ID NO: 40, or an amino acid sequence having at least about 70% sequence identity (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater) to the amino acid sequence of SEQ ID NO: 40, with an N-terminal truncation, a C-terminal truncation, an internal truncation, or a combination thereof. Non-limiting examples of full-length (e.g., natural) collagens are provided in Table 1 below.

In some cases, a polypeptide provided herein may be at least 50 amino acids, at least 75 amino acids, at least 100 amino acids, at least 125 amino acids, at least 150 amino acids, at least 175 amino acids, at least 200 amino acids, at least 225 amino acids, at least 250 amino acids, at least 275 amino acids, at least 300 amino acids, at least 350 amino acids, at least 400 amino acids, at least 450 amino acids, or at least 500 amino acids in length.

In other embodiments, polypeptides as disclosed herein may be truncated collagen polypeptides comparable to fish collagens, including from other species of sturgeon, or from other species producing roe suitable for caviar, including salmon, steelhead, trout, lumpfish, whitefish, or carp, as well as other fish such as tilapia and sharks. Suitable comparable sequences from Acipenser schrenckii (Japanese sturgeon) include NCBI accession numbers BAO58965.1, BAO58966.1, BAO58967.1, BAT51012.1, BAR72360.1, BAR72359.1, BAR72358.1, BAR72357.1 and BAR72356.1. Suitable sequences from Acipenser ruthenus (Sterlet sturgeon) include NCBI accession numbers A0A444UGW0, A0A444TZM6, A0A444UC45, A0A444UC53, A0A662YTX1, A0A662Z270, A0A662YZ39, A0A444UIF5, A0A444UJK3, A0A444UNU0, X5HZZ7, X5IHC1, A0A444UPK8, A0A444UBS1, A0A444UYQ7, A0A444TWQ3, A0A444ULY4, A0A444TZ23, A0A662YS48, A0A444U4C8, A0A444UD64, A0A662YX10, A0A662YXI2, A0A444TXQ4, A0A444TZ42, A0A444U8N8, A0A444UJU3, A0A444UQ51, A0A444U2T2, A0A662YJ50, A0A444VIV9, A0A444V113, A0A662YWR6, A0A662YW91, A0A444U5J5, A0A662YR93, A0A444UJB0, A0A444UFS4, A0A444UVK2, A0A444UJU1, A0A444ULY9, A0A444UKA7, A0A444U5L7, A0A444V6M4, A0A444V788, A0A444UFS9, A0A444UVP7, A0A444U4D9, A0A444UHN6, A0A662YJC1, A0A444VIE8, A0A444UPM0, A0A662YU87, A0A444TZS8, A0A444U200, A0A444V2E3, A0A662YXD3, A0A662YQA4, A0A444U1H9, A0A444V715, A0A444UFX8, A0A444V7B8, A0A444U2K4, A0A444V762, A0A444UQ49, A0A662YMD3, A0A662YWF2, A0A444UE44, A0A444UAR6, A0A444UX46, A0A444U5P4, A0A662YRG8, A0A444USC3, A0A444UK09, A0A444UNQ7, A0A444UN69, A0A444V5D9, E6Y298, A0A444TZY1, A0A444TYS0, and E6Y299.

In other embodiments, polypeptides may be truncated collagen polypeptides comparable to chicken collagens, or other poultry collagens, such as from domestic fowls, including chickens, turkeys, geese, and ducks. Suitable comparable sequences from Gallus gallus (chicken) include NCBI accession numbers V9GZR2, Q9PSS5, A0A3Q2UDI3, Q90802, A0A1D5PNH7, Q4TZW6, Q90803, Q91014, A0A1D5PPI0, A0A1D5P1A5, A0A3Q2U6K2, A0A3Q2U8F9, Q90689, A0A3Q2U3U6, P13731, A0A1D5PFE0, A0A3Q2TXZ7, Q5FY72, A0A1D5PR16, A0A1D5PKR6, FINDF5, Q90589, P08125, FINRH2, P32017, A0A1D5PW49, Q90800, P12108, E1C353, Q7LZR2, P02460, A0A1L1RNI7, Q90796, P12106, FINQ20, Q919K3, P20785, A0A1D5PWN6, P15988, P12105, F1NIL4, 093419, P02467, A0A5H1ZRJ7, A0A1D5PKQ4, A0A5H1ZRK9, Q90W37, A0A1D5NY11, A0A1D5P959, P02457, A0A1D5PYU1, A0A1D5PE57, Q90ZA0, Q90584, A0A1L1RZW7, A0A1D5NVM0, A0A1D5P8P3, F1NIP0, F1P2Q3, A0A1D5PE74, Q9IAU4, A0A3Q2TTC1, F1NHH4, P32018, A0A1D5P0F4, R4GHP9, A0A3Q2UD12, A0A3Q2UMJ2, A0A3Q2U4U7, F1NX22, A0A1D5P818, A0A1L1RPW4, P13944, P15989, F1P2F0, A0A1D5PGD5, and A0A3Q3AR07.

TABLE 1
Full-length collagen amino acid sequences

| Collagen | Amino Acid Sequence |
|---|---|
| Gallus gallus | MAQLLRLFQTLLILLLRDYISAEDGETR |
| (chicken) | ASCRTAPADLVFILDGSYSVGPENFEII |
| type 21 | KSWLVNITRNFDIGPKFIQVGVVQYSDY |
| alpha 1 | PVLEIPLGTHESTENLIKEMESIHYLGG |
| collagen | NTKTGRAIQFAYDHLFAKSSRFLTKIAV |
| | VLTDGKSQDEVKDVAAEARKNKITLFAI |
| | GVGSEIEEDELKAIANKPSSTYVFYVED |
| | YIAISRIKEVIKQKLCEESVCPTRIPVA |
| | ARDEKGFDILVGLGVKKRVKKRIQIPTT |
| | NAKAYEVTSRVDLSELTRNVFPEGLPPS |
| | YVFVSTQRFKVKKTWDLWRVLSLDKRPQ |
| | IAVTINGEEKTLSFTTTSLINGTQVITF |
| | AAPRVKTLFDEGWHQIRLLVTEDFVTLY |
| | IDDQEIETKPLHPVLGIYISGLTQIGKY |
| | SGKEETVQFDIQKLRIYCDPEQNNRETV |
| | CEIPGFNGECMNGPSDVGSTPAPCICPP |
| | GKQGPPGPKGDPGQPGNHGYPGQPGPDG |
| | KPGYQGSAGTPGIPGTPGVQGPRGLPGI |
| | KGEPGKDGTKGDRGLPGFPGLHGMPAPK |
| | GERGPKGDQGVPGIYGKKGSKGEKGDTG |
| | FPGMPGRSGDPGRSGKDGLPGSPGFKGE |
| | VGQPGSPGLEGHRGEPGIPGIPGNQGAK |
| | GQKGEIGPPGLPGAKGSPGETGLMGPEG |
| | SFGLPGAPGPKGDKGEPGLQGKPGSSGA |
| | KGEPGGPGAPGEPGYPGIPGTQGIKGDK |
| | GSQGESGIQGRKGEKGRQGNPGLQGTEG |
| | LRGEQGEKGEKGDPGIRGINGQKGESGI |
| | QGLVGPPGVRGQPGDRGPPGPPGSDGKP |
| | AREFSEEFIRQVCSDVLRTQLPVILQSG |
| | RLQNCNHCQSQSASPGLPGPPGPRGPEG |
| | PRGFPGLPGNDGVPGLTGIPGRPGARGT |
| | RGLPGKNGAKGNQGIGVPGIQGPPGPPG |
| | PEGPPGMSKEGRPGERGQPGKDGDRGSP |
| | GMPGPVGPPGICDPSLCFSVIVGRDPFR |
| | KGPNY |
| | (SEQ ID NO: 31) |
| Acipenser | MFSFVDSRTVLLLAAIQLCLLAVVKCQD |
| schrenckii | VEVQQPGRKGQKGEPGDITDVVGPRGPG |
| (Japanese | GPMGPPGEQGPRGERGDKGDKGGPGPRG |
| sturgeon) | RDGEPGTPGNPGPPGPPGPNGPPGLGGN |
| type 2 | FAAQMAGGFDEKAGGAQMGVMQGPMGPM |
| alpha 1 | GPRGPPGPTGAPGPQGFQGNPGEPGEPG |
| collagen | AAGPLGPRGPPGPSGKPGEDGEAGKPGK |
| | SGERGSPGPQGARGFPGTPGLPGIKGHR |
| | GYPGLDGAKGEAGAAGSKGEAGSSGENG |
| | APGPMGPRGLPGERGRNGPSGAAGARGN |
| | DGLPGPAGPPGPVGPAGAPGFPGSPGSK |
| | GEAGPTGARGPEGAQGPRGESGTPGSPG |
| | PSGASGNPGTDGIPGAKGSAGAPGIAGA |
| | PGFPGPRGPPGPQGATGPLGPKGQQGDP |
| | GIPGFKGEHGPKGEHGPAGPQGAPGPAG |
| | EEGKRGARGEPGAAGPLGPPGERGAPGN |
| | RGFPGQDGLAGPKGAPGERGQPGVGGPK |
| | GANGDPGRPGEPGLPGARGLTGRPGDAG |
| | PQGKGGPSGAAGEDGRPGPPGPQGARGQ |
| | PGVMGFPGPKGANGEPGKAGEKGLVGPP |
| | GLRGLSGKDGETGAAGPPGPSGPAGERG |
| | EQGPPGPSGFQGLPGPPGPPGEGGKPGD |
| | QGVPGEAGAAGRAGPRGERGFPGERGSP |
| | GAQGLQGPRGLPGTPGTDGPKGATGPSG |
| | ALGAQGPPGLQGMPGERGASGIAGAKGD |
| | RGDVGEKGPEGASGKDGSRGLTGPIGPP |
| | GPAGPNGEKGESGPSGPPGAAGTRGAPG |
| | DRGENGPPGPAGFAGPPGADGQPGAKGE |
| | QGEGGQKGDAGAPGPQGPSGAPGPQGPT |
| | GVSGPKGARGAQGPPGATGFPGAAGRVG |
| | PPGPNGNPGPSGPAGSAGKDGPKGVRGD |
| | AGPPGRAGDAGLQGAAGPPGEKGEPGED |
| | GPPGPDGPSGPQGLGGNRGIVGLPGQRG |
| | ERGFPGLPGPSGEPGKQGAPGGAGDRGP |
| | PGPVGPPGLSGPSGEPGREGNPGSDGPP |
| | GRDGSAGIKGDRGQTGPAGAPGAPGAPG |
| | SPGPVGPTGKQGDRGESGAQGPAGPSGP |
| | AGARGMAGPQGPRGDKGEAGETGERGQK |
| | GHRGFTGLQGLPGPPGTAGDQGAAGPAG |
| | PTGARGPPGPVGPHGKDGSNGQPGPIGP |
| | PGPRGRSGEVGPAGPPGNAGPPGPPGPP |
| | GPGIDMSAFAGLAAPEKAPDPMRYMRAD |
| | EASSSLRQHDAEVDATLKSINNQIENIR |
| | SPEGSKKNPARTCRDLKLCHPDWKSGDY |
| | WIDPNQGCAVDAIKVFCNMESGETCVYP |
| | NPASIPRKNWWTSKSADCKHVWFGETMN |
| | GGFHFSYGDDSLAPNTASIQMTFLRLLS |
| | TEASQNLTYHCKNSIAYMDQSAGNLKKA |
| | VLLQGSNDVEIRAEGNSRFTYNVLEDGC |
| | TKHTDRWGKTVIEYKSQKTSRLPIVDIA |
| | PLDIGGSDQEFGVDIGPVCY |
| | (SEQ ID NO: 32) |
| Jellyfish | GPQGVVGADGKDGTPGEKGEQGRTGAAG |
| (Hydrozoan) | KQGSPGADGARGPLGSIGQQGARGEPGD |
| collagen | PGSPGLRGDTGLAGVKGVAGPSGRPGQP |
| | GANGLPGVNGRGGLRGKPGAKGIAGSDG |
| | EAGESGAPGQSGPTGPRGQRGPSGEDGN |
| | PGLQGLPGSDGEPGEEGQPGRSGQPGQQ |
| | GPRGSPGEVGPRGSKGPSGDRGDRGERG |
| | VPGQTGSAGNVGEDGEQGGKGVDGASGP |
| | SGALGARGPPGSRGDTGAVGPPGPTGRS |
| | GLPGNAGQKGPSGEPGSPGKAGSAGEQG |
| | PPGKDGSNGEPGSPGKEGERGLAGPPGP |
| | DGRRGETGSPGIAGALGKPGLEGPKGYP |
| | GLRGRDGTNGKRGEQGETGPDGVRGIPG |
| | NDGQSGKPGIDGIDGTNGQPGEAGYQGG |
| | RGTRGQLGETGDVGQNGDRGAPGPDGSK |
| | GSAGRPGLR(SEQ ID NO: 39) |
| Human type 21 | MAHYITFLCMVLVLLLQNSVLAEDGEVR |
| alpha 1 | SSCRTAPTDLVFILDGSYSVGPENFEIV |
| collagen | KKWLVNITKNFDIGPKFIQVGVVQYSDY |
| | PVLEIPLGSYDSGEHLTAAVESILYLGG |
| | NTKTGKAIQFALDYLFAKSSRFLTKIAV |
| | VLTDGKSQDDVKDAAQAARDSKITLFAI |
| | GVGSETEDAELRAIANKPSSTYVFYVED |
| | YIAISKIREVMKQKLCEESVCPTRIPVA |
| | ARDERGFDILLGLDVNKKVKKRIQLSPK |
| | KIKGYEVTSKVDLSELTSNVFPEGLPPS |
| | YVFVSTQRFKVKKIWDLWRILTIDGRPQ |
| | IAVTLNGVDKILLFTTTSVINGSQVVTF |
| | ANPQVKTLFDEGWHQIRLLVTEQDVTLY |
| | IDDQQIENKPLHPVLGILINGQTQIGKY |
| | SGKEETVQFDVQKLRIYCDPEQNNRETA |
| | CEIPGFNGECLNGPSDVGSTPAPCICPP |
| | GKPGLQGPKGDPGLPGNPGYPGQPGQDG |
| | KPGYQGIAGTPGVPGSPGIQGARGLPGY |
| | KGEPGRDGDKGDRGLPGFPGLHGMPGSK |
| | GEMGAKGDKGSPGFYGKKGAKGEKGNAG |
| | FPGLPGPAGEPGRHGKDGLMGSPGFKGE |
| | AGSPGAPGQDGTRGEPGIPGFPGNRGLM |
| | GQKGEIGPPGQQGKKGAPGMPGLMGSNG |
| | SPGQPGTPGSKGSKGEPGIQGMPGASGL |
| | KGEPGATGSPGEPGYMGLPGIQGKKGDK |
| | GNQGEKGIQGQKGENGRQGIPGQQGIQG |
| | HHGAKGERGEKGEPGVRGAIGSKGESGV |
| | DGLMGPAGPKGQPGDPGPQGPPGLDGKP |
| | GREFSEQFIRQVCTDVIRAQLPVLLQSG |
| | RIRNCDHCLSQHGSPGIPGPPGPIGPEG |
| | PRGLPGLPGRDGVPGLVGVPGRPGVRGL |
| | KGLPGRNGEKGSQGFGYPGEQGPPGPPG |
| | PEGPPGISKEGPPGDPGLPGKDGDHGKP |
| | GIQGQPGPPGICDPSLCFSVIARRDPFR |
| | KGPNY(SEQ ID NO: 40) |

In some cases, a non-naturally occurring polypeptide (e.g., truncated collagen) as described herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 31) from amino acid positions 1 to 537; from amino acid positions 1 to 542; from amino acid positions 1 to 547; from amino acid positions 1 to 552; from amino acid positions 1 to 557; from amino acid positions 1 to 562; from amino acid positions 1 to 567; from amino acid positions 1 to 572; or from amino acid positions 1 to 577. In some cases, a non-naturally occurring polypeptide (e.g., truncated collagen) as described herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with a C-terminal truncation at any amino acid position (relative to SEQ ID NO: 31) from amino acid positions 726 to 957; from amino acid positions 731 to 957; from amino acid positions 736 to 957; from amino acid positions 741 to 957; from amino acid positions 746 to 957; from amino acid positions 751 to 957; from amino acid positions 756 to 957; from amino acid positions 761 to 957; from amino acid positions 766 to 957; from amino acid positions 769 to 957; from amino acid positions 774 to 957; from amino acid positions 779 to 957; or from amino acid positions 784 to 957. In some cases, a non-naturally occurring polypeptide as described herein (e.g., a truncated collagen) may comprise both an N-terminal truncation and a C-terminal truncation. For example, a non-naturally occurring polypeptide (e.g., truncated collagen) as described herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 31) from amino acid positions 1 to 537; from amino acid positions 1 to 542; from amino acid positions 1 to 547; from amino acid positions 1 to 552; from amino acid positions 1 to 557; from amino acid positions 1 to 562; from amino acid positions 1 to 567; from amino acid positions 1 to 572; or from amino acid positions 1 to 577; and with a C-terminal truncation at any amino acid position (relative to SEQ ID NO: 31) from amino acid positions 726 to 957; from amino acid positions 731 to 957; from amino acid positions 736 to 957; from amino acid positions 741 to 957; from amino acid positions 746 to 957; from amino acid positions 751 to 957; from amino acid positions 756 to 957; from amino acid positions 761 to 957; from amino acid positions 766 to 957; from amino acid positions 769 to 957; from amino acid positions 774 to 957; from amino acid positions 779 to 957; or from amino acid positions 784 to 957. In a specific embodiment, a non-naturally occurring polypeptide (e.g., truncated collagen) disclosed herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at amino acid position 557 (relative to SEQ ID NO: 31); and with a C-terminal truncation at amino acid position 746 (relative to SEQ ID NO: 31). In another specific embodiment, a non-naturally occurring polypeptide (e.g., truncated collagen) disclosed herein may comprise the amino acid sequence of SEQ ID NO: 31, or an amino acid sequence having at least about 80% (e.g., at least about 85%, at least about 90%, at least about 95%, at least about 98%, or greater) sequence identity thereto, with an N-terminal truncation at amino acid position 557 (relative to SEQ ID NO: 31); and with a C-terminal truncation at amino acid position 769 (relative to SEQ ID NO: 31).

In some cases, a non-naturally occurring polypeptide (e.g., truncated collagen) as described herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 32) from amino acid positions 1 to 660; from amino acid positions 1 to 665; from amino acid positions 1 to 670; from amino acid positions 1 to 675; from amino acid positions 1 to 680; from amino acid positions 1 to 685; from amino acid positions 1 to 690; from amino acid positions 1 to 695; or from amino acid positions 1 to 700. In some cases, a non-naturally occurring polypeptide (e.g., truncated collagen) as described herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with a C-terminal truncation at any amino acid position (relative to SEQ ID NO: 32) from amino acid positions 855 to 1420; from amino acid positions 860 to 1420; from amino acid positions 865 to 1420; from amino acid positions 870 to 1420; from amino acid positions 875 to 1420; from amino acid positions 880 to 1420; from amino acid positions 885 to 1420; from amino acid positions 890 to 1420; from amino acid positions 895 to 1420; or from amino acid positions 900 to 1420. In some cases, a non-naturally occurring polypeptide as described herein (e.g., a truncated collagen) may comprise both an N-terminal truncation and a C-terminal truncation. For example, a non-naturally occurring polypeptide (e.g., truncated collagen) as described herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with an N-terminal truncation at any amino acid position (e.g., relative to SEQ ID NO: 32) from amino acid positions 1 to 660; from amino acid positions 1 to 665; from amino acid positions 1 to 670; from amino acid positions 1 to 675; from amino acid positions 1 to 680; from amino acid positions 1 to 685; from amino acid positions 1 to 690; from amino acid positions 1 to 695; or from amino acid positions 1 to 700; and with a C-terminal truncation at any amino acid position (relative to SEQ ID NO: 32) from amino acid positions 855 to 1420; from amino acid positions 860 to 1420; from amino acid positions 865 to 1420; from amino acid positions 870 to 1420; from amino acid positions 875 to 1420; from amino acid positions 880 to 1420; from amino acid positions 885 to 1420; from amino acid positions 890 to 1420; from amino acid positions 895 to 1420; or from amino acid positions 900 to 1420. In a specific embodiment, a non-naturally occurring polypeptide (e.g., truncated collagen) disclosed herein may comprise the amino acid sequence of SEQ ID NO: 32, or an amino acid sequence having at least about 80% (e.g., at least about 85%, at least about 90%, at least about 95%, at least about 98%, or greater) sequence identity thereto, with an N-terminal truncation at amino acid position 680 (relative to SEQ ID NO: 32); and with a C-terminal truncation at amino acid position 880 (relative to SEQ ID NO: 32).

In some cases, a truncated collagen as described herein may comprise an internal truncation at any amino acid position between amino acid positions 16 and 240; between amino acid positions 16 and 245; between amino acid positions 16 and 250; between amino acid positions 16 and 255; between amino acid positions 16 and 260; between amino acid positions 16 and 265; between amino acid positions 6 and 255; between amino acid positions 11 and 255; between amino acid positions 21 and 255; between amino acid positions 26 and 255; between amino acid positions 31 and 255; between amino acid positions 21 and 250; between amino acid positions 21 and 245; between amino acid positions 26 and 250; between amino acid positions 26 and 245; between amino acid positions 31 and 250; or between amino acid positions 31 and 245 of SEQ ID NO: 39. In a specific embodiment, a truncated collagen as described herein may comprise an internal truncation at amino acid positions 16 and 255 of SEQ ID NO: 39.

In some cases, a truncated collagen as described herein may comprise an N-terminal truncation at any amino acid position between amino acid positions 1 and 548; between amino acid positions 1 and 553; between amino acid positions 1 and 558; between amino acid positions 1 and 563; between amino acid positions 1 and 568; or between amino acid positions 1 and 573 of SEQ ID NO: 40. In some cases, a truncated collagen as described herein may comprise a C-terminal truncation at any amino acid position between amino acid positions 726 and 957; between amino acid positions 731 and 957; between amino acid positions 736 and 957; between amino acid positions 741 and 957; between amino acid positions 746 and 957; between amino acid positions 751 and 957; or between amino acid positions 756 and 957 of SEQ ID NO: 40. In some cases, a truncated collagen as described herein may comprise both an N-terminal truncation and a C-terminal truncation. For example, a truncated collagen as described herein may comprise an N-terminal truncation at any amino acid position between amino acid positions 1 and 548; between amino acid positions 1 and 553; between amino acid positions 1 and 558; between amino acid positions 1 and 563; between amino acid positions 1 and 568; or between amino acid positions 1 and 573 of SEQ ID NO: 40; and a C-terminal truncation at any amino acid position between amino acid positions 726 and 957; between amino acid positions 731 and 957; between amino acid positions 736 and 957; between amino acid positions 741 and 957; between amino acid positions 746 and 957; between amino acid positions 751 and 957; or between amino acid positions 756 and 957. In a specific embodiment, a truncated collagen disclosed herein may comprise an N-terminal truncation at amino acid position 558 of SEQ ID NO: 40; and a C-terminal truncation at amino acid position 746 of SEQ ID NO: 40.

In some cases, a non-naturally occurring polypeptide (e.g., truncated collagen) may comprise any amino acid sequence provided herein. In some cases, a non-naturally occurring polypeptide (e.g., truncated collagen) may consist of any amino acid sequence provided herein. In some cases, a non-naturally occurring polypeptide (e.g., truncated collagen) may consist essentially of any amino acid sequence provided herein. In specific embodiments, the non-naturally occurring polypeptide has or comprises an amino acid sequence of any one of SEQ ID NO: 2, SEQ ID NO: 4, SEQ ID NO: 6, SEQ ID NO: 8, SEQ ID NO: 35, and SEQ ID NO: 37. In some embodiments, a non-naturally occurring polypeptide (e.g., truncated collagen) comprises an amino acid sequence having at least about 80%, at least about 85%, at least about 90%, at least about 95%, or at least about 98% sequence identity to any one of SEQ ID NO: 2, SEQ ID NO: 4, SEQ ID NO: 6, SEQ ID NO: 8, SEQ ID NO: 35, and SEQ ID NO: 37. In some embodiments, the non-naturally occurring polypeptide consists of or consists essentially of an amino acid sequence of any one of SEQ ID NO: 2, SEQ ID NO: 4, SEQ ID NO: 6, SEQ ID NO: 8, SEQ ID NO: 35, and SEQ ID NO: 37.

In some aspects, the non-naturally occurring polypeptide may include any chimeric collagen that includes at least one non-continuous collagen fragment. For example, the non-naturally occurring polypeptide can be a chimeric collagen in which a portion of N-terminus collagen is contiguous with a portion of C-terminus collagen where the portion of N-terminus collagen and the portion of C-terminus collagen are not contiguous in the natural or naturally-present corresponding collagens. In another example, the non-naturally occurring polypeptide can be a chimeric collagen in which a portion of C-terminus collagen is contiguous with a portion of N-terminus collagen (e.g., in a flipped or reverse order-C terminus collagen is located in the N-terminus of the portion of N-terminus collagen) where the portion of C-terminus collagen and the portion of N-terminus collagen are contiguous or non-contiguous in the natural or naturally-present corresponding collagens. In another example, the non-naturally occurring polypeptide can be a chimeric collagen in which one portion of a collagen polypeptide is contiguous with a portion of a second collagen polypeptide (e.g., a collagen molecule comprising a portion of a collagen from a first species contiguous with a portion of a collagen from a second species is a chimeric collagen, etc.).

Exemplary amino acid sequences of or nucleic acid sequences encoding the recombinant polypeptides are provided below:

A nucleotide sequence encoding a truncated
collagen type 21 alpha 1
polypeptide from Gallus gallus (chicken)
SEQ ID NO: 1
GATACCGGTTTTCCGGGTATGCCTGGTCGTAGCGGTGATCCGGGT
CGTAGCGGTAAAGATGGTCTGCCTGGTAGCCCGGGTTTTAAAGGT
GAAGTTGGTCAGCCAGGTAGCCCTGGTCTGGAAGGTCATCGTGGT
GAACCGGGTATTCCAGGTATTCCGGGTAATCAGGGTGCAAAAGGT
CAGAAAGGCGAAATTGGTCCTCCGGGTCTGCCAGGTGCCAAAGGT
TCTCCGGGTGAAACCGGTCTGATGGGTCCTGAAGGTAGCTTTGGC
CTGCCTGGTGCACCGGGTCCGAAAGGTGACAAAGGTGAACCTGGT
CTGCAGGGTAAACCGGGTAGCAGCGGTGCAAAAGGCGAACCAGGT
GGTCCGGGTGCTCCGGGTGAACCAGGCTATCCGGGTATTCCTGGT
ACTCAGGGTATTAAAGGCGATAAAGGTAGCCAGGGTGAAAGCGGT
ATTCAGGGTCGTAAGGGTGAAAAAGGCCGTCAGGGTAATCCAGGC
CTGCAGGGCACCGAAGGTCTGCGTGGCGAACAGGGCGAAAAAGGT
GAGAAGGGTGACCCAGGCATTCGT
Amino acid sequence of a truncated
collagen type 21 alpha 1 polypeptide
from Gallus gallus (chicken)
SEQ ID NO: 2
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKGDKGSQGESGIQGRKGEKGRQGNPGLQGTEGLRGEQGEKG
EKGDPGIR
A nucleotide sequence encoding a truncated
collagen type 21 alpha 1
polypeptide from Gallus gallus (chicken)
SEQ ID NO: 3
GATACTGGTTTCCCGGGGATGCCTGGGCGCTCAGGTGATCCGGGG
CGTAGTGGAAAAGACGGTCTGCCGGGGTCCCCGGGCTTTAAGGGT
GAGGTGGGTCAGCCCGGTAGTCCAGGTTTAGAAGGTCACCGCGGA
GAGCCCGGGATTCCAGGCATTCCTGGCAACCAGGGTGCCAAGGGA
CAGAAAGGCGAAATTGGTCCGCCCGGCCTACCGGGCGCGAAAGGT
TCTCCTGGTGAAACCGGTCTCATGGGTCCGGAAGGTAGCTTCGGC
CTGCCCGGCGCACCTGGTCCGAAGGGCGATAAGGGGGAGCCTGGG
CTGCAAGGTAAACCGGGTAGTTCTGGCGCCAAAGGTGAACCCGGC
GGTCCCGGTGCGCCAGGGGAACCAGGTTATCCTGGTATTCCTGGA
ACCCAAGGAATTAAAGGTGACAAAGGCTCACAGGGCGAAAGTGGT
ATACAGGGTCGCAAGGGCGAAAAAGGACGTCAGGGCAATCCAGGC
CTGCAGGGTACTGAAGGCCTGCGTGGAGAACAGGGTGAGAAAGGT
GAAAAAGGAGATCCTGGTATTCGC
Amino acid sequence of a truncated
collagen type 21 alpha 1 polypeptide
from Gallus gallus (chicken)
SEQ ID NO: 4
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKGDKGSQGESGIQGRKGEKGRQGNPGLQGTEGLRGEQGEKG
EKGDPGIR
The nucleotide sequence encoding
a truncated collagen type 21 alpha 1
polypeptide from Gallus gallus (chicken)
SEQ ID NO: 5
GATACTGGTTTCCCGGGGATGCCTGGGCGCTCAGGTGATCCGGGG
CGTAGTGGAAAAGACGGTCTGCCGGGGTCCCCGGGCTTTAAGGG
TGAGGTGGGTCAGCCCGGTAGTCCAGGTTTAGAAGGTCACCGCGG
AGAGCCCGGGATTCCAGGCATTCCTGGCAACCAGGGTGCCAAGGG
ACAGAAAGGCGAAATTGGTCCGCCCGGCCTACCGGGCGCGAAAGG
TTCTCCTGGTGAAACCGGTCTCATGGGTCCGGAAGGTAGCTTCGG
CCTGCCCGGCGCACCTGGTCCGAAGGGCGATAAGGGGGAGCCTGG
GCTGCAAGGTAAACCGGGTAGTTCTGGCGCCAAAGGTGAACCCGG
CGGTCCCGGTGCGCCAGGGGAACCAGGTTATCCTGGTATTCCTGG
AACCCAAGGAATTAAAGGTGACAAAGGCTCACAGGGCGAAAGTGG
TATACAGGGTCGCAAGGGCGAAAAAGGACGTCAGGGCAATCCAGG
CCTGCAGGGTACTGAAGGCCTGCGTGGAGAACAGGGTGAGAAAGG
TGAAAAAGGAGATCCTGGTATTCGCGGCATTAACGGTCAAAAGGG
TGAAAGTGGGATACAAGGTCTTGTCGGTCCGCCCGGAGTTAGAGG
CCAG
Amino acid sequence of a truncated
collagen type 21 alpha 1 polypeptide
from Gallus gallus (chicken)
SEQ ID NO: 6
DTGFPGMPGRSGDPGRSGKDGLPGSPGFKGEVGQPGSPGLEGHRG
EPGIPGIPGNQGAKGQKGEIGPPGLPGAKGSPGETGLMGPEGSFG
LPGAPGPKGDKGEPGLQGKPGSSGAKGEPGGPGAPGEPGYPGIPG
TQGIKGDKGSQGESGIQGRKGEKGRQGNPGLQGTEGLRGEQGEKG
EKGDPGIRGINGQKGESGIQGLVGPPGVRGQ
The nucleotide sequence encoding a
truncated collagen type 2 alpha 1
polypeptide from Acipenser schrenckii
(Japanese sturgeon)
SEQ ID NO: 7
GTCTGCAGGGTATGCCTGGTGAACGTGGTGCAAGCGGTATTGCCG
GTGCAAAAGGTGATCGTGGTGATGTTGGTGAAAAAGGTCCGGAAG
GTGCCAGCGGTAAAGATGGTAGCCGTGGTCTGACCGGTCCGATTG
GTCCGCCTGGTCCGGCAGGTCCGAATGGCGAAAAAGGTGAAAGCG
GTCCGAGCGGTCCTCCGGGTGCAGCAGGTACTCGTGGTGCACCGG
GTGATCGCGGTGAAAATGGTCCACCGGGTCCTGCCGGTTTTGCAG
GTCCGCCAGGTGCAGATGGTCAGCCTGGTGCCAAAGGCGAACAAG
GCGAAGGTGGTCAGAAAGGTGATGCAGGCGCTCCGGGTCCGCAGG
GTCCTTCTGGTGCACCTGGTCCTCAGGGTCCGACCGGTGTTTCTG
GTCCGAAAGGCGCACGTGGTGCCCAGGGTCCACCTGGTGCGACCG
GTTTTCCTGGCGCAGCAGGTCGTGTTGGTCCTCCAGGTCCTAATG
GTAATCCGGGTCCAAGCGGTCCTGCAGGTAGCGCAGGCAAAGATG
GTCCTAAAGGTGTACGCGGTGATGCTGGTCCTCCTGGCCGTGCCG
GTGATGCCGGT
Amino acid sequence of a truncated
collagen type 2 alpha 1 polypeptide from
Acipenser schrenckii (Japanese sturgeon)
SEQ ID NO: 8
GLQGMPGERGASGIAGAKGDRGDVGEKGPEGASGKDGSRGLTGPI
GPPGPAGPNGEKGESGPSGPPGAAGTRGAPGDRGENGPPGPAGFA
GPPGADGQPGAKGEQGEGGQKGDAGAPGPQGPSGAPGPQGPTGVS
GPKGARGAQGPPGATGFPGAAGRVGPPGPNGNPGPSGPAGSAGKD
GPKGVRGDAGPPGRAGDAG
The nucleotide sequence encoding a
secretion signal sequence named
Secretion Signal Sequence 1
SEQ ID NO: 9
ATGAAAAAGATTTGGCTGGCGCTGGCTGGTTTAGTTTTAGCGTTT
AGCGCATCGGCG
Amino acid sequence of a Secretion
Signal Sequence 1
SEQ ID NO: 10
MKKIWLALAGLVLAFSASA
The nucleotide sequence encoding a
secretion signal sequence named
Secretion Signal Sequence 2
SEQ ID NO: 11
ATGAAAAAAGGTTTCATGCTGTTCACCCTCCTCGCTGCGTTCTCT
GGTTTCGCGCAGGCT
Amino acid sequence of a Secretion Signal
Sequence 2
SEQ ID NO: 12
MKKGFMLFTLLAAFSGFAQA
The nucleotide sequence encoding a
secretion signal sequence named
Secretion Signal Sequence 3
SEQ ID NO: 13
ATGATGATCACCCTGCGTAAACTGCCGCTGGCTGTTGCTGTTGCT
GCTGGTGTTATGTCTGCTCAGGCTATGGCT
Amino acid sequence of a Secretion
Signal Sequence 3
SEQ ID NO: 14
MMITLRKLPLAVAVAAGVMSAQAMA
The nucleotide sequence encoding a
secretion signal sequence named
Secretion Signal Sequence 4
SEQ ID NO: 15
ATGAAAAAAACCGCTATCGCTATCGCTGTTGCTCTGGCTGGTTTC
GCTACCGTTGCTCAGGCT
Amino acid sequence of a Secretion Signal
Sequence 4
SEQ ID NO: 16
MKKTAIAIAVALAGFATVAQA
The nucleotide sequence encoding a
secretion signal sequence named
Secretion Signal Sequence 5
SEQ ID NO: 17
ATGAAAGTTAAAGTTCTGTCTCTGCTGGTTCCGGCTCTGCTGGTT
GCTGGTGCTGCTAACGCT
Amino acid sequence of a Secretion Signal
Sequence 5
SEQ ID NO: 18
MKVKVLSLLVPALLVAGAANA
The nucleotide sequence encoding a
secretion signal sequence named
Secretion Signal Sequence 6
SEQ ID NO: 19
ATGAAAAAAAACATCCTGTCTCTGTCTATGGTTGCTCTGTCTCTG
TCTCTGGCTCTGGGTTCTGTTTCTGTTACCGCT
Amino acid sequence of a Secretion
Signal Sequence 6
SEQ ID NO: 20
MKKNILSLSMVALSLSLALGSVSVTA
The nucleotide sequence encoding a
secretion signal sequence named
Secretion Signal Sequence 7
SEQ ID NO: 21
ATGCTGAACCCGAAAGTTGCTTACATGGTTTGGATGACCTGCCTG
GGTCTGACCCTGCCGTCTCAGGCT
Amino acid sequence of a Secretion Signal
Sequence 7
SEQ ID NO: 22
MLNPKVAYMVWMTCLGLTLPSQA
The nucleotide sequence encoding a
secretion signal sequence named
Secretion Signal Sequence 8
SEQ ID NO: 23
ATGAAACAGGCTCTGCGTGTAGCGTTCGGTTTCCTGATACTGTGG
GCTTCTGTTCTGCACGCT
Amino acid sequence of a Secretion Signal
Sequence 8
SEQ ID NO: 24
MKQALRVAFGFLILWASVLHA
A codon-optimized nucleotide sequence
encoding a truncated collagen type
2 alpha 1 polypeptide from
Acipenser schrenckii (Japanese sturgeon)
SEQ ID NO: 25
GGTCTGCAGGGTATGCCGGGTGAACGTGGTGCCAGCGGTATTGCA
GGTGCCAAAGGTGATCGTGGTGATGTTGGTGAAAAAGGTCCGGAA
GGTGCAAGCGGTAAAGATGGTAGCCGTGGTCTGACCGGTCCGATT
GGTCCGCCGGGTCCGGCCGGTCCGAATGGTGAAAAAGGTGAAAGC
GGTCCGAGCGGTCCGCCGGGTGCAGCCGGTACCCGTGGTGCACCG
GGTGATCGTGGTGAAAATGGTCCGCCGGGTCCGGCCGGTTTTGCA
GGTCCGCCGGGTGCCGATGGTCAGCCGGGTGCAAAAGGTGAACAG
GGTGAAGGTGGTCAGAAAGGTGATGCCGGTGCACCGGGTCCGCAG
GGTCCGAGCGGTGCCCCGGGTCCGCAGGGTCCGACCGGTGTTAGC
GGTCCGAAAGGTGCACGTGGTGCCCAGGGTCCGCCGGGTGCAACC
GGTTTTCCGGGTGCCGCAGGTCGTGTTGGTCCGCCGGGTCCGAAT
GGTAATCCGGGTCCGAGCGGTCCGGCAGGTAGCGCCGGTAAAGAT
GGTCCGAAAGGTGTTCGTGGTGATGCAGGTCCGCCGGGTCGTGCC
GGTGATGCAGGTTAA
A codon-optimized nucleotide sequence
encoding a truncated collagen type
2 alpha 1 polypeptide from
Acipenser schrenckii (Japanese sturgeon)
SEQ ID NO: 26
GGCCTGCAAGGCATGCCAGGCGAGCGCGGCGCGTCTGGCATCGCG
GGCGCGAAGGGCGACCGCGGCGACGTGGGCGAGAAGGGCCCTGAG
GGCGCGTCCGGCAAGGACGGCTCTCGCGGCCTGACAGGCCCAATC
GGCCCTCCAGGCCCTGCGGGCCCAAACGGCGAGAAGGGCGAGTCC
GGCCCTTCTGGCCCACCTGGCGCGGCGGGCACACGCGGCGCGCCA
GGCGACCGCGGCGAGAACGGCCCTCCAGGCCCTGCGGGCTTCGCG
GGCCCACCTGGCGCGGACGGCCAACCAGGCGCGAAGGGCGAGCAA
GGCGAGGGCGGCCAAAAGGGCGACGCGGGCGCGCCTGGCCCACAA
GGCCCTTCTGGCGCGCCAGGCCCTCAAGGCCCAACAGGCGTGTCC
GGCCCTAAGGGCGCGCGCGGCGCGCAAGGCCCACCTGGCGCGACA
GGCTTCCCAGGCGCGGCGGGCCGCGTGGGCCCTCCAGGCCCTAAC
GGCAACCCAGGCCCTTCTGGCCCAGCGGGCTCCGCGGGCAAGGAC
GGCCCTAAGGGCGTGCGCGGCGACGCGGGCCCACCTGGCCGCGCG
GGCGACGCGGGCTGA
A codon-optimized nucleotide sequence
encoding a truncated collagen type
2 alpha 1 polypeptide from
Acipenser schrenckii (Japanese sturgeon)
SEQ ID NO: 27
GGTTTGCAAGGTATGCCAGGGGAACGGGGTGCGTCCGGGATAGCC
GGGGCAAAAGGTGATCGAGGCGATGTAGGAGAAAAAGGCCCAGAA
GGGGCGTCAGGTAAGGACGGATCTCGCGGCTTGACGGGACCTATC
GGGCCTCCAGGTCCCGCCGGCCCTAATGGGGAAAAAGGCGAGAGT
GGGCCGTCTGGTCCGCCCGGCGCCGCTGGCACACGTGGAGCGCCG
GGCGATCGTGGTGAGAACGGACCACCGGGTCCTGCTGGTTTTGCG
GGACCTCCGGGAGCAGACGGCCAGCCGGGCGCTAAAGGTGAACAG
GGTGAAGGTGGCCAAAAAGGCGATGCAGGCGCACCGGGTCCGCAG
GGCCCTTCAGGTGCACCGGGTCCACAGGGCCCAACTGGCGTTTCA
GGGCCGAAAGGCGCAAGAGGTGCTCAGGGTCCGCCCGGGGCAACT
GGGTTTCCTGGAGCGGCCGGCCGTGTTGGACCTCCGGGGCCGAAC
GGAAACCCTGGACCGTCTGGACCAGCCGGTTCAGCGGGTAAGGAT
GGTCCTAAGGGTGTAAGGGGTGACGCAGGTCCCCCTGGACGTGCA
GGGGATGCGGGGTAG
A codon-optimized nucleotide sequence
encoding a truncated collagen type
2 alpha 1 polypeptide from
Acipenser schrenckii (Japanese sturgeon)
SEQ ID NO: 28
GGGTTACAAGGTATGCCGGGAGAACGTGGAGCGTCAGGAATTGCT
GGGGCCAAAGGTGATCGTGGTGATGTTGGCGAGAAAGGGCCCGAA
GGCGCATCTGGTAAAGATGGCTCACGCGGGTTAACTGGACCAATC
GGACCACCAGGCCCCGCTGGGCCTAATGGTGAAAAGGGTGAAAGT
GGCCCTTCTGGACCCCCAGGAGCCGCCGGTACACGTGGAGCGCCA
GGCGATCGTGGCGAAAACGGACCGCCCGGACCTGCAGGTTTTGCG
GGACCCCCTGGAGCAGACGGCCAACCAGGAGCAAAAGGTGAGCAA
GGTGAAGGTGGACAAAAGGGAGATGCCGGAGCGCCAGGCCCCCAA
GGCCCATCAGGAGCTCCAGGACCTCAAGGTCCAACTGGTGTATCA
GGGCCTAAGGGTGCGCGCGGCGCTCAAGGACCGCCTGGCGCAACT
GGCTTTCCGGGAGCTGCTGGTCGTGTGGGCCCGCCTGGCCCAAAC
GGAAATCCAGGCCCTTCAGGCCCGGCGGGCTCAGCCGGAAAAGAC
GGTCCGAAGGGAGTCCGTGGAGATGCGGGACCGCCAGGACGCGCT
GGCGATGCAGGCTAA
A codon-optimized nucleotide sequence
encoding a truncated collagen type
2 alpha 1 polypeptide from
Acipenser schrenckii (Japanese sturgeon)
SEQ ID NO: 29
GGTTTACAGGGAATGCCAGGGGAACGCGGCGCCTCAGGGATTGCC
GGTGCTAAAGGAGATCGTGGCGACGTGGGTGAAAAGGGTCCCGAG
GGAGCATCAGGTAAGGATGGTTCCCGTGGTTTGACGGGACCTATT
GGACCTCCGGGTCCTGCAGGTCCGAACGGCGAAAAGGGGGAAAGC
GGGCCTAGTGGTCCACCCGGCGCCGCAGGTACCCGTGGTGCCCCA
GGCGACCGCGGGGAGAATGGACCGCCTGGCCCTGCCGGTTTTGCG
GGTCCTCCAGGAGCCGATGGGCAGCCCGGTGCAAAAGGAGAGCAG
GGAGAGGGAGGTCAAAAGGGAGATGCCGGCGCCCCGGGCCCTCAG
GGACCAAGCGGTGCGCCAGGCCCCCAGGGTCCTACGGGTGTTAGC
GGGCCGAAAGGCGCACGCGGAGCGCAGGGCCCACCTGGTGCAACA
GGCTTCCCAGGAGCTGCGGGGCGCGTCGGACCTCCGGGACCCAAT
GGAAACCCAGGTCCGTCAGGGCCGGCAGGCTCCGCAGGGAAAGAT
GGTCCCAAAGGCGTGCGTGGAGACGCAGGGCCCCCCGGACGCGCC
GGCGATGCGGGATAA
A codon-optimized nucleotide sequence
encoding a truncated collagen type
21 polypeptide from Gallus gallus
SEQ ID NO: 30
GATACTGGTTTCCCGGGGATGCCTGGGCGCTCAGGTGATCCGGGG
CGTAGTGGAAAAGACGGTCTGCCGGGGTCCCCGGGCTTTAAGGGT
GAGGTGGGTCAGCCCGGTAGTCCAGGTTTAGAAGGTCACCGCGGA
GAGCCCGGGATTCCAGGCATTCCTGGCAACCAGGGTGCCAAGGGA
CAGAAAGGCGAAATTGGTCCGCCCGGCCTACCGGGCGCGAAAGGT
TCTCCTGGTGAAACCGGTCTCATGGGTCCGGAAGGTAGCTTCGGC
CTGCCCGGCGCACCTGGTCCGAAGGGCGATAAGGGGGAGCCTGGG
CTGCAAGGTAAACCGGGTAGTTCTGGCGCCAAAGGTGAACCCGGC
GGTCCCGGTGCGCCAGGGGAACCAGGTTATCCTGGTATTCCTGGA
ACCCAAGGAATTAAAGGTGACAAAGGCTCACAGGGCGAAAGTGGT
ATACAGGGTCGCAAGGGCGAAAAAGGACGTCAGGGCAATCCAGGC
CTGCAGGGTACTGAAGGCCTGCGTGGAGAACAGGGTGAGAAAGGT
GAAAAAGGAGATCCTGGTATTCGC
Amino acid sequence of a truncated collagen
polypeptide from jellyfish
SEQ ID NO: 35
GPQGVVGADGKDGTPGNAGQKGPSGEPGSPGKAGSAGEQGPPGKD
GSNGEPGSPGKEGERGLAGPPGPDGRRGETGSPGIAGALGKPGLE
GPKGYPGLRGRDGTNGKRGEQGETGPDGVRGIPGNDGQSGKPGID
GIDGTNGQPGEAGYQGGRGTRGQLGETGDVGQNGDRGAPGPDGSK
GSAGRPGLR
A codon-optimized nucleotide sequence
encoding a truncated collagen
polypeptide from jellyfish
SEQ ID NO: 36
GGTCCGCAGGGTGTTGTTGGTGCAGATGGTAAAGACGGTACCCCG
GGTAATGCAGGTCAGAAAGGTCCGTCAGGTGAACCTGGCAGCCC
TGGTAAAGCAGGTAGTGCCGGTGAGCAGGGTCCGCCGGGCAAAGA
TGGTAGTAATGGTGAGCCGGGTAGCCCTGGCAAAGAAGGTGAACG
TGGTCTGGCAGGACCGCCGGGTCCTGATGGTCGCCGCGGTGAAAC
GGGTTCACCGGGTATTGCCGGTGCCCTGGGTAAACCAGGTCTGGA
AGGTCCGAAAGGTTATCCTGGTCTGCGCGGTCGTGATGGTACCAA
TGGCAAACGTGGCGAACAGGGCGAAACCGGTCCAGATGGTGTTCG
TGGTATTCCGGGTAACGATGGTCAGAGCGGTAAACCGGGCATTGA
TGGTATTGATGGCACCAATGGTCAGCCTGGCGAAGCAGGTTATCA
GGGTGGTCGCGGTACCCGTGGTCAGCTGGGTGAAACAGGTGATGT
TGGTCAGAATGGTGATCGCGGCGCACCGGGTCCGGATGGTAGCAA
AGGTAGCGCCGGTCGTCCGGGTTTACGTTAA
Amino acid sequence of a truncated type 21
alpha 1 collagen polypeptide from human
SEQ ID NO: 37
AGFPGLPGPAGEPGRHGKDGLMGSPGFKGEAGSPGAPGQDGTRGE
PGIPGFPGNRGLMGQKGEIGPPGQQGKKGAPGMPGLMGSNGSPGQ
PGTPGSKGSKGEPGIQGMPGASGLKGEPGATGSPGEPGYMGLPGI
QGKKGDKGNQGEKGIQGQKGENGRQGIPGQQGIQGHHGAKGERGE
KGEPGVR
A codon-optimized nucleotide sequence
encoding a truncated type 21 alpha
1 collagen polypeptide from human
SEQ ID NO: 38
TGCAGGTTTTCCGGGTCTGCCTGGTCCGGCAGGCGAACCGGGTCG
TCATGGTAAAGATGGTCTGATGGGTAGTCCGGGTTTTAAAGGTGA
AGCAGGTTCACCGGGTGCACCTGGTCAGGATGGCACCCGTGGTGA
ACCGGGTATTCCGGGATTTCCGGGTAATCGTGGCCTGATGGGTCA
GAAAGGTGAAATTGGTCCGCCTGGTCAGCAGGGTAAAAAAGGCGC
ACCGGGTATGCCAGGACTGATGGGTTCAAATGGCAGTCCGGGTCA
GCCAGGCACACCGGGTTCAAAAGGTAGCAAAGGCGAACCTGGTAT
TCAGGGTATGCCTGGTGCAAGCGGTCTGAAAGGCGAGCCAGGTGC
CACCGGTTCTCCGGGTGAACCAGGTTATATGGGTCTGCCAGGTAT
CCAAGGCAAAAAAGGTGATAAAGGTAATCAGGGCGAAAAAGGCAT
TCAGGGCCAGAAAGGCGAAAATGGCCGTCAGGGTATTCCAGGCCA
GCAGGGCATCCAGGGTCATCATGGTGCAAAAGGTGAACGTGGTGA
AAAGGGCGAACCAGGTGTTCGTTAA

In some embodiments, the non-naturally occurring polypeptide comprises an amino acid sequence of any one of SEQ ID NOs: 2, 4, 6, 8, 35, and 37. In some embodiments, the non-naturally occurring polypeptide comprises an amino acid sequence having a sequence identity of at least about 80%, at least about 85%, at least about 90%, at least about 95%, or at least about 98% thereof, or the like, to the amino acid sequence of any one of SEQ ID NOs: 2, 4, 6, 8, 35, and 37. Alternatively and/or additionally, the non-naturally occurring polypeptide is encoded by a nucleic acid sequence of any one of SEQ ID NOs: 1, 3, 5, 7, 25-30, 36, and 38. In some embodiments, the non-naturally occurring polypeptide is encoded by a nucleic acid having sequence identity of at least about 65%, at least about 70%, at least about 75%, at least about 80%, at least about 85%, at least about 90%, at least about 95%, or at least about 98% thereof, or the like, to the nucleic acid sequence of any one of SEQ ID NOs: 1, 3, 5, 7, 25-30, 36, and 38.

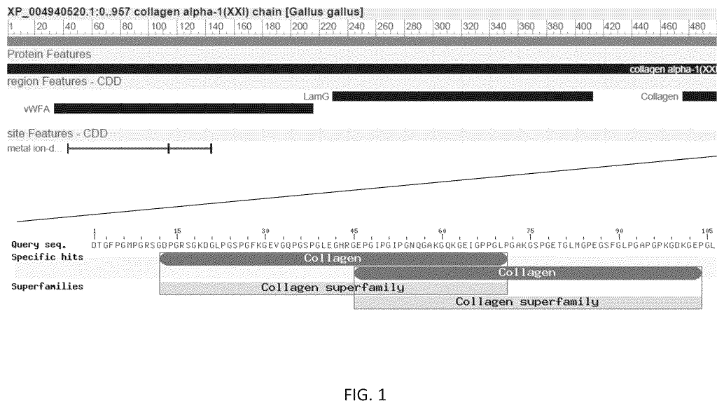

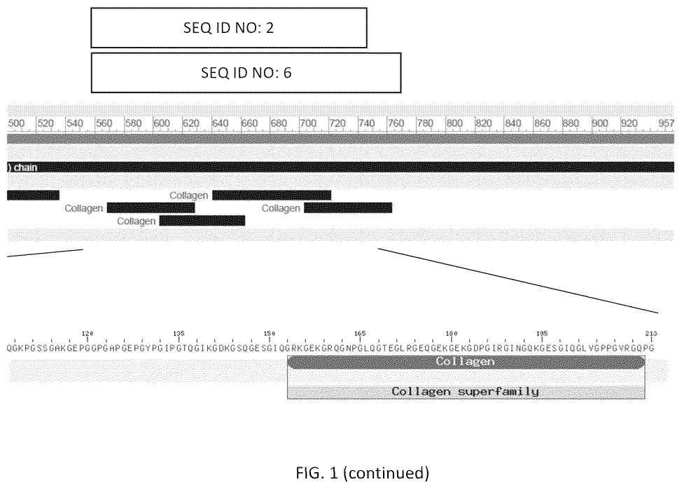

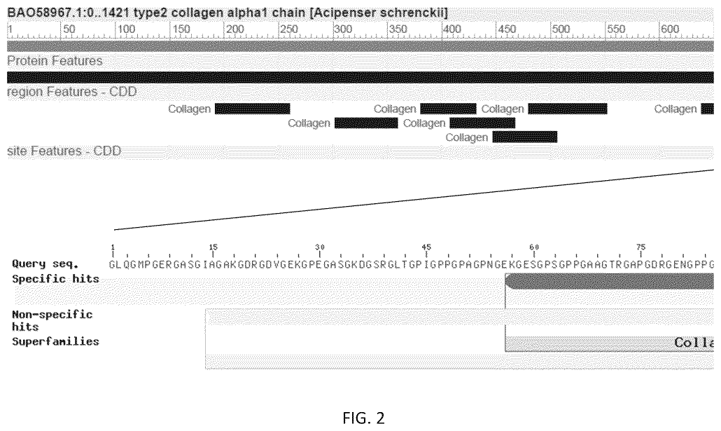

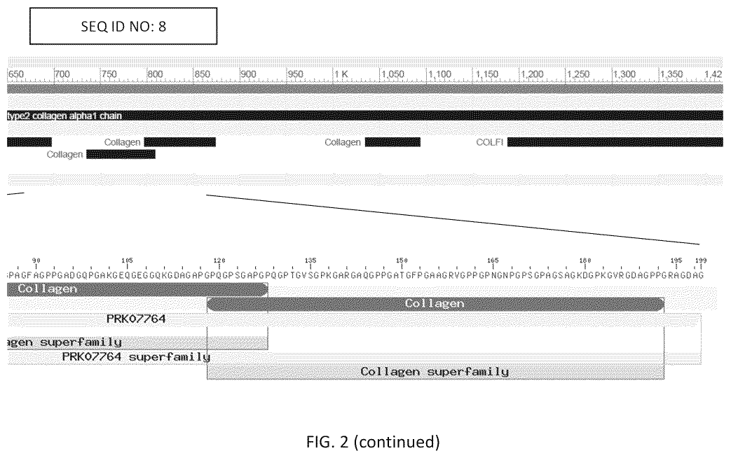

In some aspects, the non-naturally occurring polypeptides provided herein may or may not contain one or more domains from natural collagen. FIG. 1 and FIG. 2 depict an alignment of exemplary non-naturally occurring polypeptides (e.g., truncated collagens) of the disclosure with the corresponding naturally occurring collagen. FIG. 1 depicts an alignment of a non-naturally occurring polypeptide of SEQ ID NO: 2 and SEQ ID NO: 6 with Gallus gallus type 21 alpha 1 collagen. FIG. 2 depicts an alignment of a non-naturally occurring polypeptide of SEQ ID NO: 8 with Acipenser schrenckii type 2 alpha 1 collagen. FIG. 1 and FIG. 2 demonstrate that non-naturally occurring polypeptides may have one or more domains found in natural collagen (e.g., collagen triple helix repeat domains). FIG. 1 and FIG. 2 further demonstrate that non-naturally occurring polypeptides may lack one or more domains found in natural collagen (e.g., Von Willebrand factor type A (vWA) domain, laminin G domain, fibrillar collagen C-terminal domain). In some aspects, a non-naturally occurring polypeptide provided herein may contain one or more collagen triple helix repeat domains. In some aspects, a non-naturally occurring polypeptide provided herein may lack one or more of a Von Willebrand factor type A (vWA) domain, a laminin G domain, and a fibrillar collagen C-terminal domain).

In some embodiments, the non-naturally occurring polypeptide (e.g., recombinant polypeptide) includes a secretion signal sequence. Any suitable secretion signal sequence (e.g., hydrophobic signaling peptides, Sec signal peptides, Tat signal peptides, etc.) that can induce the non-naturally occurring polypeptide (e.g., recombinant polypeptide) to be secreted to the periplasmic and/or extracellular space (e.g., when produced in a recombinant host cell). Exemplary secretion signal sequences include a peptide having an amino acid sequence of any one of SEQ ID NOs: 10, 12, 14, 16, 18, 20, 22, and 24. Alternatively and/or additionally, the secretion signal sequence includes a peptide encoded by a nucleic acid sequence of any one of SEQ ID NOs: 9, 11, 13, 15, 17, 19, 21, and 23. The secretion signal sequence is preferably located at the N-terminus of the non-naturally occurring polypeptide (e.g., recombinant polypeptide). Yet, it is contemplated that the secretion signal sequence can be located at other than N-terminus where the secretion signal sequence remains functional.

The non-naturally occurring polypeptide (e.g., recombinant polypeptide) as described herein can be expressed or generated via a nucleic acid sequence encoding the non-naturally occurring polypeptide (e.g., recombinant polypeptide). Thus, another aspect of the disclosure includes an expression vector comprising a nucleic acid sequence encoding the non-naturally occurring polypeptide (e.g., recombinant polypeptide). In some embodiments, the expression vector is a bacterial expression vector. In some embodiments, the expression vector is a yeast expression vector. In some embodiments, the expression vector is an insect expression vector. Any suitable expression vector that can induce the protein expression from the inserted nucleic acid encoding the non-naturally occurring polypeptide (e.g., recombinant polypeptide). Exemplary bacterial expression vectors may include pGEX vectors where glutathione S-transferase is used as a fusion partner and gene expression is under the control of the tac promoter, or pET vectors (e.g., pET28 vector, etc.) which uses a T7 promoter. Exemplary yeast expression vectors may include pPIC vectors, which uses the AOX1 promoter inducible with methanol. In some embodiments, the expression vector is in a plasmid form (e.g., including bacterial artificial chromosome form, etc.) that are independently present in the host cell (e.g., cells expressing the recombinant polypeptide). In some embodiments, the expression vector is stably integrated into the chromosome of the host cell via random or targeted integration.

In some embodiments, the nucleic acid sequence encoding the non-naturally occurring polypeptide (e.g., recombinant polypeptide) is codon-optimized to be expressed in non-animal cells, preferably in bacterial cells. As used herein, “codon-optimized” means that the codon composition is improved for expression in the heterologous cells (e.g., microbial cells, bacterial cells, etc.) without altering the encoded amino acid sequences. Non-limiting examples of codon-optimized nucleic acid sequences (e.g., encoding a non-naturally occurring polypeptide as described herein) include SEQ ID NOs: 25-30, 36, and 38.

In some embodiments, the expression vector may include one or more selection agent. The selection agents include certain sugars including galactose containing sugars or antibiotics including ampicillin, hygromycin, G418 and others. Enzymes that are used to confer resistance to the selection agent include β-galactosidase or a β-lactamase. Alternatively and/or additionally, the expression vector includes an inducible promoter or a constitutive promoter (e.g., CMV promoter, etc.) such that the nucleic acid encoding the recombinant protein is operatively linked to the inducible promoter or the constitutive promoter. For example, the expression vector may include tetracycline-inducible promoter pTET, araC-ParaBAD inducible promoter, or IPTG inducible lac promoter. As used herein, “operatively linked” promoter and nucleic acid means that the expression of the nucleic acid (e.g., transcription, translation, etc.) is at least under partial control of the promoter.

In some embodiments, the nucleic acid encoding the non-naturally occurring polypeptide (e.g., recombinant polypeptide) (e.g., a nucleic acid of any one of SEQ ID NOs: 1, 3, 5, 7, 25-30, 36, and 38), and the expression vector may have an overlap of from 20 to 50 bp long, from 20 to 40 bp long, from 20 to 30 bp long, or from 30 to 40 bp long. Such overlap can be added using PCR with a DNA polymerase (e.g., PRIMESTAR® GXL polymerase (takarabio.com/products/pcr/gc-rich-pcr/primestar-gxl-dna-polymerase)). Opened expression vector and the insert nucleic acid encoding the non-naturally occurring polypeptide (e.g., recombinant polypeptide) can be assembled together into the final plasmid using any suitable cloning system (e.g., IN-FUSION® Cloning (takarabio.com/products/cloning/in-fusion-cloning) or SGI Gibson assembly (us.vwr.com/store/product/17613857/gibson-assembly-hifi-1-step-kit-synthetic-genomics-inc)).

Such prepared expression vector (or plasmid) can be used to generate genetically engineered or modified organisms, or a recombinant cell to produce the non-naturally occurring polypeptides described herein (e.g., collagens, truncated collagens, or collagen fragments). Preferably, the recombinant cells contain at least one copy of a plasmid or a stably integrated heterologous nucleic acid sequence encoding the non-naturally occurring polypeptide (e.g., collagens, truncated collagens, or collagen fragments, preferably collagens, truncated collagens, or collagen fragments of, or derived from, Gallus gallus collagen and/or Acipenser schrenckii collagen). In some embodiments, the recombinant cell is a microbial cell. For example, where the expression vector is bacterial expression vector, the expression vector can be inserted into (e.g., via any suitable transformation method) the bacterial cells for protein expression (e.g., Escherichia coli including BL-21 cells, etc.) to be independently present in the cytoplasm of the bacteria (e.g., as a plasmid form) or to be at least temporarily and/or stably integrated into the bacterial chromosome.

Consequently, the transformed cells can be cultivated in a suitable media. Preferably, the suitable media includes a minimal media and the cells are frozen in 1.5 aliquots with vegetable glycerin at a ratio of 50:50 of cells of cells to glycerin. For protein expression, one vial of the frozen cultured cells can be cultured in a suitable amount of bacteria culture media (e.g., minimal media, 50 mL, 100 mL, etc.) for at least 6 hours, at least 8 hours, at least 10 hours, at least 12 hours, at least overnight at least 36° C., preferably at about 37° C. by continuously shaking the culture (e.g., at least 100 rpm, at least 200 rpm, at least 250 rpm, etc.). Table 2 and Table 3 show the exemplary formulation of the minimal media that can be used for cell cultivation and culture.

TABLE 2
Minimal Media Formulation

| 1) Autoclave 5 L of 550 g/kg Glucose syrup at | |
|---|---|
| concentration in DI water. (VWR, product #97061-170). | |
| 2) Autoclave | 20 g (NH₄)₂HPO₄. (VWR, product # 97061-932). |
| in 3946 mL | 66.5 g KH₂PO₄. (VWR, product # 97062-348). |
| of DI water | 22.5 g H₃C₆H₅O₇. (VWR, product #BDH9228-2.5 |
| and add | KG). |
| | 8.85 g MgSO₄•7H₂O. (VWR, product # 97062-134). |
| | 10 mL of 1000x Trace metals formulation |

After autoclaving, add:
118 g of (1) to (2)
5 mL of 25 mg/mL Kanamycin Sulfate (VWR-V0408)
Use 28% NH₄OH (VWR, product #BDH3022) to adjust pH to 6.1.

TABLE 3
Trace metals formulation

| | |
|---|---|
| Ferrous Sulfate Heptahydrate | 27.8 g/L (Spectrum, 7782-63-0) |
| Zinc Sulfate heptahydrate | 2.88 g/L (Spectrum, 7446-20-0) |
| Calcium chloride dihydrate | 2.94 g/L (Spectrum, 2971347) |
| Sodium molybdate dihydrate | 0.48 g/L (Spectrum, 10102-40-6) |
| Manganese chloride tetrahydrate | 1.26 g/L (Spectrum, 13446-34-9) |
| Sodium selenite | 0.35 g/L (Spectrum, 10102-18-8) |
| Boric acid | 0.12 g/L (Spectrum, 10043-35-3) |

In some embodiments, transformed cells can then be transferred to a larger volume of growth media (e.g., minimal media) and grown for at least 4 hours, at least 5 hours, at least 6 hours, at least 7 hours, at least 8 hours, from 5 to 10 hours, from 5 to 9 hours, from 6 to 9 hours, and/or alternatively until the cell density in the media reaches optical density (OD) of 600.

Additionally, fermentation process can be performed at various temperature ranging from 22° C. to 33° C., from 29° C. to 33° C., from 30° C. to 32° C., from 23° C. to 29° C., or from 25° C. to 28° C. In some embodiments, the temperature of the fermentation can be maintained at a constant temperature and immediately upon completion of fermentation the non-naturally occurring polypeptide can be purified. Alternatively, the temperature of the fermentations can be maintained for a desired period of time and when cell densities of OD600 of 10-20 are reached, then the temperature can be reduced to induce protein production. In such embodiments, typically, the temperature is reduced from 28° C. to 25° C. During the fermentation, protein expression in the bacteria can be induced by adding induction reagent. For example, where the expression vector contains lac promoter and the nucleic acid encoding the non-naturally occurring polypeptide (e.g., truncated collagen, collagen fragments, or collagen) is under the control of the lac promoter, the expression of the nucleic acid can be induced by adding isopropyl β-d-1-thiogalactopyranoside (IPTG) at a concentration ranging from 0.1-1.5 mM, from 0.1-1.0 mM, or from 0.1-0.5 mM. Fermentation can be continued for 20-24 hours, or in some embodiments, for 40-60 hours.

It is contemplated that such generated recombinant cells (e.g., recombinant bacteria transformed with the expression vector) intracellularly express the non-naturally occurring polypeptides (e.g., truncated collagen, collagen fragments, or collagen) encoded by the nucleic acids in the expression vector. Such intracellularly expressed polypeptides (e.g., truncated collagen, collagen fragments, or collagen) can then be secreted (via a secretion signal sequence) to the extracellular space (e.g., into a culture media). Thus, in some embodiments, the culture media can contain secreted recombinant protein (e.g., truncated collagen, collagen fragments, or collagen) encoded by the nucleic acids.

Thus, another aspect of the disclosure includes a composition including the non-naturally occurring polypeptide (e.g., recombinant collagen, truncated collagen, collagen fragments, or collagen) encoded by the nucleic acids. In some embodiments, the composition may include the recombinant cell comprising an integrated heterologous nucleic acid sequence encoding a non-naturally occurring polypeptide (e.g., collagen, a truncated collagen, or fragment thereof), and/or the culture medium (e.g., growth media, cultivation media, etc.) for the recombinant cell.

Alternatively and/or additionally, the composition may include purified recombinant polypeptides from the recombinant cells and/or the culture medium. In some embodiments, the recombinant polypeptides are purified from the culture medium where the recombinant cells grow and secrete the recombinant polypeptides thereto. In some embodiments, the recombinant polypeptide is coupled with a tag (e.g., histidine tag, etc) such that the recombinant polypeptide can be purified using affinity purification is known as immobilized metal affinity chromatography (IMAC). Alternatively, the recombinant polypeptide can be purified via column chromatography. For example, the recombinant polypeptide can be purified by acid treatment of homogenized growth media. In such example, the pH of the growth media (e.g., fermentation broth) can be decreased to from 3 to 3.5 using 5-50% sulfuric acid. The recombinant cells are then separated using centrifugation. Supernatant of the acidified broth can be tested on a polyacrylamide gel and determined whether it contains the recombinant polypeptide in relatively high abundance compared to starting pellet. The recombinant polypeptide slurry obtained is generally high in salts. To obtain volume and salt reduction, concentration and diafiltration steps can be performed using filtration steps. For example, the filtration step can be performed using EMD Millipore Tangential Flow Filtration system with ultrafiltration cassettes of 0.1 m² each. Total area of filtration in this example can be 0.2 m² using two cassettes in parallel. A volume reduction of 5× and a salt reduction of 19× can be achieved in the TFF stage. Final slurry can be run on an SDS-PAGE gel to confirm presence of the recombinant polypeptide. The purified recombinant polypeptide can then be analyzed on an SDS-PAGE gel to identify a corresponding thick and clear band observed at the expected sizes for each respective polypeptide. Quantification of titers and purity can be further conducted using reverse phase and size exclusion HPLC chromatography. It is preferred that the purity of the purified recombinant polypeptides is at least about 80%, at least about 85%, at least about 90%, at least about 95%, or at least about 99%.

Provided herein are pharmaceutical compositions. The pharmaceutical compositions may, in some instances, comprise a polypeptide of the disclosure. In some cases, the polypeptide comprises or consists of an amino acid sequence having at least about 80% (e.g., at least about 85%, at least about 90%, at least about 95%, at least about 98%, or about 100%) sequence identity to SEQ ID NO: 32. In some cases, the polypeptide comprises or consists of an amino acid sequence having at least about 80% (at least about 85%, at least about 90%, at least about 95%, at least about 98%, or about 100%) sequence identity to a truncate of SEQ ID NO: 32 (e.g., comprising an N-terminal truncation, a C-terminal truncation, an internal truncation, or any combination thereof, as described herein).

In certain embodiments, the pharmaceutical composition may comprise one or more pharmaceutically acceptable excipients or carriers. In some cases, the pharmaceutically acceptable excipient or carrier is selected from the group consisting of: an antiadherent, a binder, a coating, a color, a disintegrant, a flavor, a glidant, a lubricant, a preservative, a sorbent, a vehicle, and any combination thereof.

Wound care in patients and animals is a common clinical challenge faced by the healthcare and veterinary industries. Wounds may include, for example, trauma wounds, burns, ulcers, lesions, abscesses, diabetic wounds, pressure sores or ulcers, and grafts or wounds resulting from surgical procedures and operations. Wounds may result from physical injury, surgical procedures and operations, heat or chemical burns, pressure on the skin, radiation, infections, immune system deficiencies, malnourishment, as well as various medical conditions such as vascular disorders and diabetes. Wounds also include more superficial wounds, for example, cuts, lacerations, punctures, grazes, scratches, abrasions, friction wounds (e.g., rash, friction blisters, and the like), boils, skin eruptions, blemishes, acne, psoriasis, eczema, oral wounds, and skin or corneal lesions.

Collagen is known to improve wound healing and stimulate tissue growth and is well-tolerated at the wound site. In particular, collagen is thought to aid in the migration of fibroblasts and keratinocytes to the wound site thereby improving tissue growth in the wound bed.

In some embodiments, the pharmaceutical composition including the non-naturally occurring polypeptides (e.g., recombinant polypeptides and/or purified recombinant polypeptides) can be formulated for topical application. The topical application can be for therapeutic purpose (e.g., for the treatment of a skin condition, as described herein). The topical formulation can be any type of topical formulation, including, but not limited to, a powder, a cream, a gel, a gel cream, a liquid, a lotion, an oil, a serum, a paste, an ointment, a medicated stick, a balm, a solution, a suspension, a foam, and the like. In further embodiments, the topical formulation may comprise the non-naturally occurring polypeptides in the form of a personal care product, such as a mask, a skin cleaner, a cleansing cream, a cleansing lotion, a facial lotion, a body lotion, a shower gel, an antiperspirant, a deodorant, a shave cream, a depilatory, a face oil, a lip oil, a body oil, a facial cleanser, a cleansing milk, a cleansing pad, a facial wash, a facial cream, a body cream, a facial moisturizer, a body moisturizer, a facial serum, a facial mask, a body mask, a facial toner, a facial mist, a foundation, a concealer, or a tinted multifunctional cream. In such embodiments, the composition may further include at least one of a carrier molecule (e.g., vehicle), a preservative, and/or additional ingredients. Any suitable carrier molecules are contemplated, and the exemplary carrier molecule may include water, oil, alcohol, propylene glycol, or emulsifiers. In addition, any suitable preservatives are contemplated, and the exemplary preservatives include zinc oxide, parabens, formaldehyde releasers, isothiazolinones, phenoxyethanol, or organic acids such as benzoic acid, sodium benzoate, or butylene glycol, hexanediol, or potassium sorbate.

In some embodiments, the pharmaceutical composition including the non-naturally occurring polypeptides (e.g., recombinant polypeptides and/or purified recombinant polypeptides) can be formulated as an injectable formulation (e.g., for administration by injection). The injectable formulation can be for therapeutic purpose (e.g., for the treatment of a skin condition, as described herein). The pharmaceutical compositions provided herein can be formulated for parental injection, including formulations suitable for bolus injection or continuous infusion. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the polypeptides disclosed herein in water soluble form. In additional embodiments, suspensions of the polypeptides are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the polypeptides to allow for the preparation of highly concentrated solutions. Alternatively, in other embodiments, the polypeptide is in powder form for constitution with a suitable vehicle, e.g., sterile pyrogen-free water, before use.

In certain embodiments, provided herein are methods of treating a wound in a subject. The methods may comprise administering a therapeutically effective amount of a polypeptide of the disclosure (e.g., in a pharmaceutical composition) to the subject to treat the wound. In some cases, the wound exhibits impaired wound healing. In some cases, the methods comprise administering the polypeptide (e.g., in a pharmaceutical composition, in a topical formulation) to the wound or to skin adjacent to the wound.

In some embodiments, the composition can be administered in and about connective tissue to add volume, add support, or otherwise treat a connective tissue condition, in addition to boosting collagen expression. The compositions described herein can be administered at multiple levels beneath the dermis. Generally, connective tissue fibers and matrix are synthesized by specialized cells called fibroblasts. There are three main groups of connective tissues: loose connective tissue, dense connective tissue, and specialized connective tissue. As used herein, the term “connective tissue” may refer to those tissues that connect, support, or surround other structures and organs of the body. For example, connective tissues described herein may include, without limitation, skin, dermal tissues, subdermal tissues, cutaneous tissues, subcutaneous tissues, intradural tissue, muscles, tendons, ligaments, cartilage, bone, fibrous tissues, adipose tissues, blood vessels and arteries, nerves, and synovial (intradermal) tissues.

A connective tissue condition refers to any condition that involves abnormalities in connective tissue in one or more parts of the body. Certain disorders are characterized by over-activity of the immune system with resulting inflammation and systemic damage to the tissues, usually with replacement of normal tissue (e.g., normal tissue of a certain organ) with connective tissue. Other disorders involve biochemical abnormalities or structural defects of the connective tissue itself. Some of these disorders are inherited, and some are of unknown etiology. When connective tissue diseases are of autoimmune origin they are classified as “rheumatic disorders”, “autoimmune rheumatic disorders” or “autoimmune collagen-vascular disorders”.

In some embodiments the compositions provided herein may be used to treat connective tissue conditions such as fibrosis or sclerosis. The term “fibrosis” as used herein refers to the accumulation of connective tissue or fibrous tissue (scar tissue, collagen) in a certain organ or part of the body. If fibrosis arises from a single cell line it is called a “fibroma”. Fibrosis occurs as the body attempts to repair and replace damaged cells, and thus can be a reactive, benign or a pathological state. Physiological fibrosis is similar to the process of scarring. A pathological state develops when the tissue in question is repeatedly and continuously damaged. A single episode of injury, even if severe, does not usually cause fibrosis. If injury is repeated or continuous (for instance as it occurs in chronic hepatitis) the body attempts to repair the damage, but the attempts result instead in excessive accumulation of scar tissue. Scar tissue starts to replace regular tissue of the organ which performs certain functions that the scar tissue is not able to perform; it can also interfere with blood flow and limit blood supply to other cells. As a result, these other functional cells start to die and more scar tissue is formed. The term “sclerosis” refers to the hardening or stiffening of tissue or a structure or organ that would normally be flexible, usually by replacement of normal organ specific tissue with connective tissue.

In certain embodiments, provided herein are methods of treating a proliferative disorder of the skin of a subject. The methods may comprise administering a therapeutically effective amount of a polypeptide of the disclosure (e.g., in a pharmaceutical composition) to the subject to treat the proliferative skin disorder. In some cases, the proliferative skin disorder is characterized by abnormal (e.g., reduced, decreased) proliferation, migration, and/or adhesion of skin cells (e.g., keratinocytes, fibroblasts). In some cases, the proliferative skin disorder is selected from the group consisting of: epidermal thinning, epidermal atrophy, dermal atrophy, epidermal degeneration, acantholysis, pemphigus foliaceus, pemphigus vulgaris, acantholytic dyskeratosis, Darier disease, Hailey-Hailey disease, Grover disease, lichen sclerosus, hyalinisation of collagen, or a combination thereof.

In some embodiments, the compositions provided herein may be used to treat conditions including, but not limited to, arthritis, skin lesion, acne vulgaris, cystic acne, psoriasis, ichthyoses (e.g., ichthyosis hystrix, epidermolytic hyperkeratosis, and lamellar ichthyosis), follicular disorders (e.g., pseudofolliculites, senile comedones, nevus comidonicas, and trichostatis spinulosa), benign epithelial tumors (e.g., flat warts, trichoepithelioma, and molluscum contagiosum), perforated dematoses (e.g., elastosis perforans seripiginosa and Kyrles disease), and disorders of keratinization (e.g., Dariers disease, keratoderma, hyperkeratosis plantaris, pityriasis rubra pilaris, lichen planus acanthosis nigricans, and psoriasis).

In various aspects, after administration of the compositions (e.g., pharmaceutical compositions) or formulations (e.g., to the skin of a subject), keratinocyte growth (e.g., proliferation) is increased. In some cases, keratinocyte growth (e.g., proliferation) is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), keratinocyte regeneration is increased. In some cases, keratinocyte regeneration is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), collagen production by fibroblasts is increased. In some cases, collagen production by fibroblasts is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), fibroblast migration is increased. In some cases, fibroblast migration is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), fibroblast proliferation is increased. In some cases, fibroblast proliferation is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), fibroblast adhesion is increased. In some cases, fibroblast adhesion is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), keratinocyte viability is increased. In some cases, keratinocyte viability is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In various aspects, after administration of the compositions (e.g., pharmaceutical compositions) or formulations (e.g., to the skin of a subject), expression of one or more genes (e.g., one or more genes involved in cell proliferation, cell migration, cell adhesion, etc.) (by a cell present in the skin, e.g., keratinocytes, fibroblasts) is increased. In some cases, expression of one or more genes (e.g., one or more genes involved in cell proliferation, cell migration, cell adhesion, etc.) (by a cell present in the skin, e.g., keratinocytes, fibroblasts) is increased by at least about 10%, at least about 20%, at least about 30%, at least about 40%, at least about 50%, at least about 60%, at least about 70%, at least about 80%, at least about 90%, at least about 100%, or greater, after the compositions (e.g., pharmaceutical compositions) or formulations are administered to a subject (e.g., as compared to prior to administration of the compositions or formulations).

In some embodiments, the one or more genes are involved in a signaling pathway (e.g., involved in cell proliferation, cell migration, cell adhesion). In some cases, the one or more genes are involved in a VEGFA/VEGFR2 signaling pathway. In some cases, the one or more genes involved in a VEGFA/VEGFR2 signaling pathway is selected from the group consisting of: MYOC1, FLII, ROCK1, ROCK2, CLTC, LIMK 1, EGR1, and any combination thereof.

In some cases, the one or more genes are involved in a focal adhesion signaling pathway. In some cases, the one or more genes involved in a focal adhesion signaling pathway is selected from the group consisting of: ITGA3, TNC, LAMC1, FLNA, TLN1, ZYX, DIAPH1, and any combination thereof.

In some cases, the one or more genes are involved in an endothelin signaling pathway. In some cases, the one or more genes involved in an endothelin signaling pathway is selected from the group consisting of: TRIOBP, WNK1, MMP2, VCAN, ACTA2, GNA12, EGR1, and any combination thereof.

In some cases, the one or more genes are involved in an EGF/EGFR signaling pathway. In some cases, the one or more genes involved in an EGF/EGFR signaling pathway is selected from the group consisting of: ATXN2, JAK1, RPS6KA2, ROCK1, SHC1, IQGAP1, PLCG1, and any combination thereof.

In some cases, the one or more genes are involved in a transforming growth factor-beta (TGF-beta) signaling pathway. In some cases, the one or more genes involved in a TGF-beta signaling pathway is selected from the group consisting of: SMURF1, SPTBN1, PAK2, ROCK1, SHC1, TGFBR3, TGFBR1, and any combination thereof.

Provided in certain embodiments herein are compositions (e.g., pharmaceutical compositions) and formulations (e.g., topical formulations) comprising one or more non-naturally occurring polypeptide provided herein (e.g., for therapeutic use). In some embodiments, the compositions and formulations provide a therapeutically effective amount of polypeptide provided herein (e.g., an amount suitable to provide a therapeutic benefit when administered to an individual or a cell). In some specific embodiments, the compositions (e.g., pharmaceutical compositions) and formulations (e.g., topical formulations) comprise an amount suitable to provide a therapeutic beneficial effect to the skin of an individual when administered to the skin of the individual. In specific embodiments, the compositions (e.g., pharmaceutical compositions) and/or formulations (e.g., topical formulations) comprise about 0.001% to about 30% w/w of a polypeptide (or non-naturally occurring collagen polypeptide) such as provided herein. In more specific embodiments, the compositions (e.g., pharmaceutical compositions) and/or formulations (e.g., topical formulations) comprise about 0.001% to about 20% w/w of a polypeptide (or non-naturally occurring collagen polypeptide) such as provided herein, about 0.001% to about 10% w/w of a polypeptide (or non-naturally occurring collagen polypeptide) such as provided herein, about 0.001% to about 5% w/w of a polypeptide (or non-naturally occurring collagen polypeptide) such as provided herein, about 0.001% to about 4% w/w of a polypeptide (or non-naturally occurring collagen polypeptide) such as provided herein, about 0.001% to about 3% w/w of a polypeptide (or non-naturally occurring collagen polypeptide) such as provided herein, about 0.001% to about 2% w/w of a polypeptide (or non-naturally occurring collagen polypeptide) such as provided herein, about 0.001% to about 1% w/w of a polypeptide (or non-naturally occurring collagen polypeptide) such as provided herein, about 0.001% to about 0.5% w/w of a polypeptide (or non-naturally occurring collagen polypeptide) such as provided herein, and about 0.001% to about 0.2% w/w of a polypeptide (or non-naturally occurring collagen polypeptide) such as provided herein.

In various embodiments, the concentration or amount of a non-naturally occurring polypeptide (e.g., recombinant protein) provided herein is in a composition (e.g., pharmaceutical composition) and/or formulation (e.g., topical formulation) provided herein in any suitable amount and may, e.g., vary depending on the use or formulation (e.g., gel, capsule, liquid, powder, etc.). Exemplary concentrations of the non-naturally occurring polypeptides (e.g., recombinant proteins) in the compositions (e.g., pharmaceutical compositions) and/or formulations (e.g., topical formulations) can be at least about 0.01%, at least about 0.05%, at least about 0.1%, at least about 0.2%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% (w/v or w/w). Alternatively and/or additionally, the exemplary concentration of the non-naturally occurring polypeptides (e.g., recombinant proteins) in the compositions (e.g., pharmaceutical composition) and/or formulations (e.g., topical formulations) can be about 0.01%, about 0.05%, about 0.1%, about 0.2%, about 0.5%, about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98% (w/v or w/w). Alternatively and/or additionally, the exemplary concentration of the non-naturally occurring polypeptides (e.g., recombinant proteins) in the compositions (e.g., pharmaceutical composition) and/or formulations (e.g., topical formulations) can range from about 0.01% to about 99%, from about 0.05% to about 99%, from about 0.1% to about 99%, from about 0.1% to about 99%, from about 0.5% to about 99%, from about 0.1% to about 10%, from about 1% to about 99%, from about 5% to about 99%, from about 10% to about 99%, from about 15% to about 99%, from about 20% to about 99%, from about 25% to about 99%, from about 30% to about 99%, from about 35% to about 99%, from about 40% to about 99%, from about 45% to about 99%, from about 50% to about 99%, from about 55% to about 99%, from about 60% to about 99%, from about 65% to about 99%, from about 70% to about 99%, from about 75% to about 99%, from about 80% to about 99%, from about 85% to about 99%, from about 90% to about 99%, from about 95% to about 99%, from about 0.1% to about 90%, from about 1% to about 90%, from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, from about 80% to about 90%, from about 85% to about 90%, from about 20% to about 80%, from about 25% to about 80%, from about 30% to about 80%, from about 35% to about 80%, from about 40% to about 80%, from about 45% to about 80%, from about 50% to about 80%, from about 55% to about 80%, from about 60% to about 80%, from about 65% to about 80%, from about 70% to about 80%, from about 75% to about 80%, from about 70% to about 99%, from about 75% to about 99%, from about 80% to about 99%, etc (w/w or w/v). Alternatively and/or additionally, the exemplary concentration of the non-naturally occurring polypeptides (e.g., recombinant proteins) in the in the compositions (e.g., pharmaceutical composition) and/or formulations (e.g., topical formulations) can be less than about 95%, about 90%, about 85%, about 80%, about 75%, about 70%, about 65%, about 60%, about 55%, about 50%, about 45%, about 40%, etc (w/w or w/v).

In some embodiments, the schedule of application varies depending on the purpose, gender, age, or health condition of the subject. For example, in some embodiments, the in the compositions (e.g., pharmaceutical composition) and/or formulations (e.g., topical formulations) are administered (e.g., topically) once a day, twice a day, three times a day, up to 6 times a day, every 2 days, every 3 days, every 4 days, every 5 days, every 6 days, etc. Alternatively and/or additionally, in some embodiments, the in the compositions (e.g., pharmaceutical composition) and/or formulations (e.g., topical formulations) are administered (e.g., topically) a plurality of times in an irregular interval, or increased interval, or decreased interval. In certain embodiments, in the compositions (e.g., pharmaceutical composition) and/or formulations (e.g., topical formulations) are (e.g., topically) administered in a dose and/or schedule sufficient or effective to provide a therapeutic benefit (e.g., as described herein).

### EXAMPLES

### Example 1. Generation of Non-Naturally Occurring Polypeptides of the Disclosure

This example shows the generation of a recombinant polypeptide of the disclosure by genetically engineered microorganisms and the purification process of such generated polypeptides.

The polynucleotides of SEQ ID NOs: 1, 3, 5, and 7 were synthesized and at least one of the polynucleotides were inserted into a pET vector. Overlaps between a pET vector and SEQ ID NOs: 1, 3, 5, and 7 were designed to be between 20 and 30 bp long and added using PCR with the enzyme PRIMESTAR® GXL polymerase (takarabio.com/products/pcr/gc-rich-pcr/primestar-gxl-dna-polymerase). The opened pET vector and insert DNA (e.g., polynucleotide of SEQ ID NO: 1) were assembled together into the final plasmid using IN-FUSION® Cloning (takarabio.com/products/cloning/in-fusion-cloning). In all cases, the nucleic acid sequences were preceded by a secretion signal sequence disclosed as SEQ ID NOs: 9, 11, 13, 15, 17, 19, 21, or 23. Plasmid sequences were verified through Sanger sequencing.

Cells were transformed with final plasmids and subsequently cultivated in minimal media and frozen in 1.5 aliquots with vegetable glycerin at a ratio of 50:50 of cells to glycerin. One vial of this frozen culture was revived in 50 ml of minimal media overnight at 37° C., 200 rpm. Formulations of the minimal media in this example are shown in Table 2 and Table 3. Cells were then transferred into 300 ml of minimal media and grown for 6-9 hours to reach an optical density (OD) 600 of 5-10.

The fermentations were performed at various temperature ranging from 25° to 28° C. For some fermentations, the temperature of the fermentation was maintained at a constant temperature and immediately upon completion of fermentation the polypeptide was purified. For other fermentations, the temperature of the fermentations was maintained for a desired period of time and when cell densities of OD600 of 10-20 were reached, the temperature was reduced to induce protein production. Typically, the temperature was reduced from 28° C. to 25° C. Induction was carried out by adding IPTG to the media at concentrations ranging from 0.1-0.5 mM. Fermentations were continued for 40-60 hours.

The recombinant polypeptide was purified as follows: The pH of the fermentation broth was decreased to between 3-3.5 using 5-50% sulfuric acid. The cells were then separated using centrifugation or centrifugation followed by microfiltration. Supernatant of the acidified broth was tested on a polyacrylamide gel and found to contain recombinant polypeptide in relatively high abundance compared to starting pellet. To obtain volume and salt reduction, concentration and diafiltration steps were performed ultrafiltration. Final polypeptide slurry was run on an SDS-PAGE gel to confirm presence of the recombinant polypeptide.

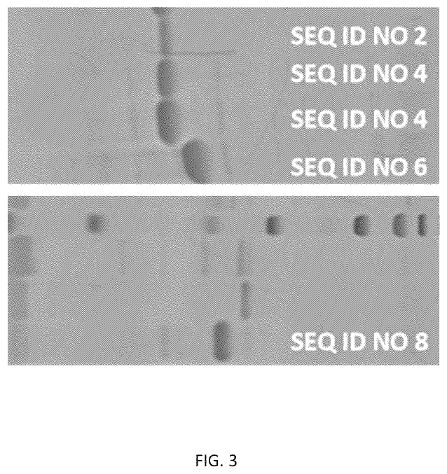

To verify that the desired proteins were produced, supernatants from cultures of microbes carrying SEQ ID NOs: 1, 3, 5, or 7 were collected and purified by decreasing their pH as described above. The acidified broth was analyzed by SDS-PAGE, and bands corresponding to the expected size protein were detected in relative purity. As shown in FIG. 3, a thick and clear band was observed at the expected sizes for each respective protein. Samples were subsequently analyzed for quantifying recombinant polypeptide titers and purity by reverse phase and size exclusion HPLC chromatography and mass spectrometry, which confirmed the correct identity of the respective proteins of interest.

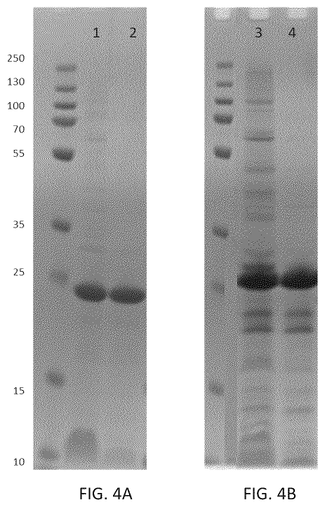

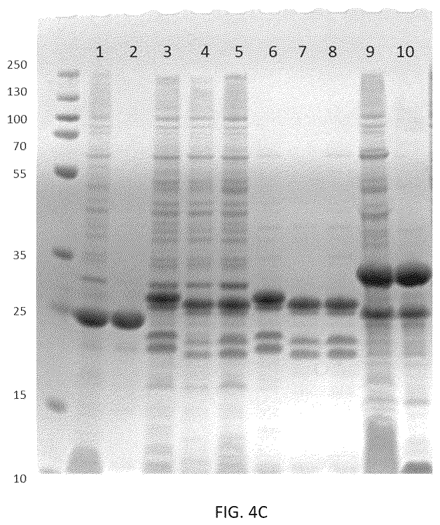

FIGS. 4A-4C depict SDS-PAGE gels of non-naturally occurring polypeptides of the disclosure before and after treatment at pH 3.0. FIG. 4A depicts an SDS-PAGE gel of fermentation supernatant containing a non-naturally occurring polypeptide having an amino acid sequence of SEQ ID NO: 2 before (Lane 1) and after (Lane 2) treatment at pH 3.0. The expected molecular weight of such polypeptide was about 17.9 kDa. The identity of the polypeptide was confirmed by mass spectrometry (data not shown). FIG. 4B depicts an SDS-PAGE gel of fermentation supernatant containing a non-naturally occurring polypeptide having an amino acid sequence of SEQ ID NO: 8 before (Lane 3) and after (Lane 4) treatment at pH 3.0. The expected molecular weight of such polypeptide was about 17.6 kDa. The identity of the polypeptide was confirmed by mass spectrometry (data not shown). FIG. 4C depicts an SDS-PAGE gel of fermentation supernatant containing a non-naturally occurring polypeptide produced in various bacterial host strains having an amino acid sequence of SEQ ID NO: 8 before (Lanes 3-5) and after (Lanes 6-8) treatment at pH 3.0.

### Example 2. Polypeptide Sequence Confirmation of Products and Lack of Hydroxyproline Residues

Mass spectrometry was used to confirm the sequence of a polypeptide of SEQ ID NO: 2 produced by methods according to this disclosure. Table 4 and Table 5 provide the results of peptide mapping of this polypeptide.

TABLE 4
Peptide mapping of the polypeptide of SEQ ID NO: 2.

| | SEQ | | | | Calculated | Observed | Mass | Retention |
|---|---|---|---|---|---|---|---|---|
| | ID | | | | Mass | Mass | Error | Time |
| Label | NO: | Sequence | Range | | (Da) | (Da) | (Da) | (min) |
| T1-Mox | 41 | DTGFPGMPGR | 1 | 10 | 1049.4501 | 1049.4598 | −0.000216 | 7.49 |
| T1-2-clipD | 42 | DTGFPGMPGR | 1 | 13 | 1292.5455 | 1292.5471 | 0.001373 | 9.13 |
| | | SD | | | | | | |
| T1-2 | 43 | DTGFPGMPGR | 1 | 16 | 1602.7209 | 1602.7206 | −0.00029 | 8.12 |
| | | SDPGR | | | | | | |
| T1-3 | 44 | DTGFPGMPGR | 1 | 19 | 1874.8694 | 1874.8634 | −0.005825 | 7.19 |
| | | SDPGRSGK | | | | | | |
| T1-4 | 45 | DTGFPGMPGR | 1 | 29 | 2830.8457 | 2830.8513 | 0.005645 | 8.6 |
| | | SDPGRSGKDG | | | | | | |
| | | LPGSPGFK | | | | | | |
| T1-clipT | 46 | TGFPGMPGR | 2 | 10 | 918.43817 | 918.438148 | 0.000225 | 8.38 |
| T1-3-clipG | 47 | GFPGMPGRSD | 3 | 19 | 1658.7947 | 1658.7977 | 0.003076 | 7.19 |
| | | PGRSGK | | | | | | |
| T1-3-clipP2 | 48 | PGMPGRSDPG | 5 | 19 | 1454.7048 | 1454.7067 | 0.001883 | 7.19 |
| | | RSGK | | | | | | |
| T1-3-clipP1 | 49 | PGRSDPGRSG | 8 | 19 | 1169.5801 | 1169.5885 | −0.001693 | 7.19 |
| | | K | | | | | | |
| T2 | 50 | SDPGR | 11 | 16 | 588.2736 | Not detected | | |
| T2-4 | 51 | SDPGRSGKDG | 11 | 29 | 1814.8911 | 1814.8878 | −0.003236 | 6.62 |
| | | LPGSPGFK | | | | | | |
| T3 | | SGK | 17 | 19 | 291.1663 | Not detected | | |
| T4 | 52 | DGLPGSPGFK | 20 | 29 | 974.4941 | Not detected | | |
| T4-5-clipE | 53 | DGLPGSPGFK | 20 | 31 | 1159.5509 | 1159.5509 | 3.40E−05 | 8.58 |
| | | GE | | | | | | |
| T4-5 | 54 | DGLPGSPGFK | 20 | 44 | 2431.188 | 2431.197 | 0.008649 | 8.48 |
| | | GEVGQPGSPG | | | | | | |
| | | LEGHR | | | | | | |
| T4-6 | 55 | DGLPGSPGFK | 20 | 59 | 3803.8979 | 3803.9045 | 0.00648 | 9.39 |
| | | GEVGQPGSPG | | | | | | |
| | | LEGHRGEPGI | | | | | | |
| | | PGIPGNDGAK | | | | | | |
| T4-7 | 56 | DGLPGSPGFK | 20 | 62 | 4117.0728 | 4117.0747 | 0.002786 | 8.83 |
| | | GEVGQPGSPG | | | | | | |
| | | LEGHRGEPGI | | | | | | |
| | | PGIPGNDGAK | | | | | | |
| | | GQK | | | | | | |
| T4-6-clipG | 57 | GLPGSPGFKG | 21 | 59 | 3688.8711 | 3688.8735 | 0.002824 | 9.01 |
| | | EVGQPGSPGL | | | | | | |
| | | EGHRGEPGIP | | | | | | |
| | | GIPGNDGAK | | | | | | |
| T4-6-clipP | 58 | FGSFGFKGEV | 29 | 44 | 2146.0557 | 2146.0562 | 0.000427 | 8.47 |
| | | GQPGSPGLEG | | | | | | |
| | | HR | | | | | | |
| T5 | 59 | GEVGQPGSPG | 30 | 44 | 1476.7189 | Not detected | | |
| | | LEGHR | | | | | | |
| T5-6 | 60 | GEVGQPGSPG | 30 | 59 | 2848.4216 | 2848.4216 | −5.61E−05 | 8.28 |
| | | LEGHRGEPGI | | | | | | |
| | | PGIPGNQGAK | | | | | | |
| T5-7 | 61 | GEVGQPGSPG | 30 | 62 | 3161.5967 | 3161.5986 | 0.00197 | 7.68 |
| | | LEGHRGEPGI | | | | | | |
| | | PGIPGNQGAK | | | | | | |
| | | GQK | | | | | | |
| T5-6-clipR | 62 | RGEPGIPGIP | 44 | 59 | 1946.8215 | 1946.822 | 0.00052 | 7.68 |
| | | GNQGAK | | | | | | |
| T6 | 63 | GEPGIPGIPG | 45 | 59 | 1891.7277 | Not | | |
| | | NQGAK | | | | detected | | |
| T6-7 | 64 | RGEPGIPGIP | 45 | 62 | 1708.8955 | 1708.8969 | 0.001454 | 7.58 |
| | | GNQGAKGQK | | | | | | |
| T6-8 | 65 | GEPGIPGIPG | 45 | 74 | 2777.4824 | 2777.48 | −0.002657 | 9.09 |
| | | NQGAKGQKGE | | | | | | |
| | | IGPPGLPGAK | | | | | | |
| T6-9 | 66 | GEPGIPGIPG | 45 | 98 | 4955.5239 | 4955.5317 | 0.008078 | 10.72 |
| | | NQGAKGQKGE | | | | | | |
| | | IGPPGLPGAK | | | | | | |
| | | GAPGFKGDKG | | | | | | |
| | | EPGLQGKFGS | | | | | | |
| | | SGAK | | | | | | |
| T6-7-clipP | 67 | PGNQGAKGQK | 53 | 62 | 983.51483 | 983.51324 | −0.001589 | 8.83 |
| T6-8-clipP | 68 | PGNQGAKGQK | 53 | 74 | 2057.1018 | 2057.1095 | 0.003865 | 9.41 |
| | | GEIGPPGLPG | | | | | | |
| | | AK | | | | | | |
| T7 | | GQK | 50 | 62 | 332.1928 | Not detected | | |
| T7-8-MetI | 69 | GQKGEIGPPG | 50 | 74 | 1418.7882 | 1418.79 | 0.002054 | 8.04 |
| | | LPGAK | | | | | | |
| T7-8-clipK | 70 | KGEIGPPGLP | 62 | 74 | 1219.6925 | 1219.691 | −0.001366 | 7.46 |
| | | GAK | | | | | | |
| T8 | 71 | GEIGPPGLPG | 63 | 74 | 1092.6047 | Not detected | | |
| | | AK | | | | | | |
| T8-11 | 72 | GEIGPPGLPG | 63 | 116 | 4920.4717 | 4920.4771 | 0.005323 | 9.79 |
| | | AKGSPGETGL | | | | | | |
| | | MGPEGSFGLP | | | | | | |
| | | GAPGPKGDKG | | | | | | |
| | | EPGLQGKPGS | | | | | | |
| | | SGAK | | | | | | |
| T8-clipI | 73 | IGPPGLPGAK | 65 | 74 | 905.53345 | 905.53308 | −0.00042 | 8.71 |
| T8-cation | 74 | GSPGETGLMG | 75 | 98 | 2234.0081 | 2233.9998 | −0.008257 | 11.12 |
| | | PEGSFGLPGA | | | | | | |
| | | PGPK | | | | | | |
| T9 | 75 | GSPGETGLMG | 75 | 98 | 2197.0593 | Not detected | | |
| | | PEGSFGLPGA | | | | | | |
| | | PGPK | | | | | | |
| T9-10-clipD | 76 | GSPGETGLMG | 75 | 100 | 2368.1006 | 2368.1003 | −0.000254 | 11.24 |
| | | PEGSFGLPGA | | | | | | |
| | | PGPKGD | | | | | | |
| T9-10 | 77 | GSPGETGLMG | 75 | 101 | 2496.1956 | 2496.1887 | −0.006757 | 10.11 |
| | | PEGSFGLPGA | | | | | | |
| | | PGPKGDK | | | | | | |
| T9-clipT | 78 | TGLMGPEGSF | 80 | 98 | 1768.8818 | 1768.88 | −0.001801 | 11.1 |
| | | GLPGAPGPK | | | | | | |
| T9-clipG | 79 | LMGPEGSFGL | 81 | 98 | 1667.8341 | 1667.8339 | −0.00081 | 11.1 |
| | | PGAPGPK | | | | | | |
| T9-clipS | 80 | SFGLPGAPGP | 88 | 98 | 1026.5498 | 1026.5485 | −0.00134 | 8.97 |
| | | K | | | | | | |
| T10-11 | 81 | GDKGEPGLQG | 99 | 116 | 1668.8431 | 1668.8383 | −0.004932 | 5.46 |
| | | KPGSSGAK | | | | | | |
| T11-11- | 82 | KGEPGLQGKP | 101 | 116 | 1496.7947 | 1496.7939 | −0.000828 | 5.39 |
| | | GSSGAK | | | | | | |
| T11 | 83 | GEPGLQGKPG | 102 | 116 | 1369.707 | Not detected | | |
| | | SSGAK | | | | | | |

TABLE 5
Peptide mapping of the polypeptide of SEQ ID NO: 2.

| | SEQ | | | | | Observed | Mass | | |
|---|---|---|---|---|---|---|---|---|---|
| | ID | | | | Calculated | Mass | Error | Time | Intensity |
| Label | NO: | Sequence | Range | | Mass (Da) | (Da) | (Da) | (min) | (counts) |
| T11-13 | 84 | GEPGLQGKPG | 102 | 145 | 3839.908 | 3839.9158 | 0.007516 | 7.99 | 15,528.34 |
| | | SSGAKGEPGG | | | | | | | |
| | | PGAPGEPGYP | | | | | | | |
| | | GIPGTQGIKG | | | | | | | |
| | | DK | | | | | | | |
| T12 | 85 | GEPGGPGAPG | 117 | 140 | 2189.0752 | 2189.0723 | −0.003083 | 9.65 | 145,576.50 |
| | | EPGYPGIPGT | | | | | | | |
| | | QGIKGDK | | | | | | | |
| T12-13 | 86 | GEPGGPGAPG | 117 | 143 | 2489.2188 | 2489.219 | 0.000101 | 8.74 | 45,482.75 |
| | | EPGYPGIPGT | | | | | | | |
| | | QGIKGDKGDK | | | | | | | |
| T12-14-clipP4 | 87 | PGEPGYPGIP | 125 | 154 | 2923.4424 | 2923.4473 | 0.005102 | 7.56 | 18,260.02 |
| | | GTQGIKGDKG | | | | | | | |
| | | SQGESGIQGR | | | | | | | |
| T12-14-CipP3 | 88 | PGYPGIPGTQ | 128 | 154 | 2648.3257 | 2648.6252 | 0.000495 | 7.58 | 16,118.55 |
| | | GIKGDKGSQG | | | | | | | |
| | | ESGIQGR | | | | | | | |
| T12-14-clipP2 | 89 | PGIPGTQGIK | 131 | 154 | 2323.188 | 2323.1851 | −0.0029 | 8.83 | 16,247.45 |
| | | GDKGSQGESG | | | | | | | |
| | | IQGR | | | | | | | |
| T12-14-ClipP1 | 90 | PGTQGIKGDK | 134 | 154 | 2056.0298 | 2056.0305 | 0.000825 | 7.56 | 24,994.64 |
| | | GSQGESGIQG | | | | | | | |
| | | R | | | | | | | |
| T12-15-ClipP | 91 | PGTQGIKGDK | 134 | 155 | 2056.0298 | 2056.0308 | 0.00072 | 8.33 | 13,644.56 |
| | | GSQGESGIQG | | | | | | | |
| | | RK | | | | | | | |
| T13 | | GDK | 141 | 143 | 319.1612 | Not detected | | | |
| T13-14 | 92 | GDKGSQGESG | 141 | 154 | 1374.6488 | 1374.6508 | 0.001375 | 3.23 | 66,051.58 |
| | | IQGR | | | | | | | |
| T13-15 | 93 | GDKGSQGESG | 141 | 155 | 1374.6488 | 1374.6508 | 0.003875 | 3.23 | 66,051.58 |
| | | IQGRK | | | | | | | |
| T13-16 | 94 | GDKGSQGESG | 141 | 158 | 1816.9027 | 1816.899 | −0.00382 | 2.88 | 2,494.46 |
| | | IQGRKGEK | | | | | | | |
| T13-17 | 95 | GDKGSQGESG | 141 | 160 | 2030.0253 | 2030.022 | −0.003288 | 2.72 | 2,147.75 |
| | | IQGRKGEKGR | | | | | | | |
| T13-14-clipK | 96 | KGSQGESGIQ | 143 | 154 | 1202.6003 | 1202.5985 | −0.00199 | 3.44 | 2,745.49 |
| | | GR | | | | | | | |
| T14 | 97 | GSQGESGIQG | 144 | 154 | 1075.5126 | Not detected | | | |
| | | R | | | | | | | |
| T14-15 | 98 | GSQGESGIQG | 144 | 155 | 1202.6003 | 1202.5985 | −0.00199 | 5.44 | 2,745.49 |
| | | RK | | | | | | | |
| T14-16 | 99 | GSQGESGIQG | 144 | 158 | 1516.7594 | 1516.7587 | −0.000831 | 3.02 | 1,968.24 |
| | | RKGEK | | | | | | | |
| T14-17 | 100 | GSQGESGIQG | 144 | 160 | 1729.882 | 1729.8776 | −0.00445 | 2.79 | 2,134.92 |
| | | RKGEKGR | | | | | | | |
| T15 | | K | 155 | 155 | 147.1128 | Not detected | | | |
| T15-18 | 101 | KGEKGRQGNP | 155 | 178 | 1981.0453 | 1981.0385 | −0.006898 | 5.56 | 19,503.56 |
| | | GLQGTEGLR | | | | | | | |
| T15-19 | 102 | KGEKGRQGNP | 155 | 179 | 2609.3269 | 2809.3287 | −0.000542 | 5.51 | 112,503.40 |
| | | GLQGTEGLRG | | | | | | | |
| | | EQGEK | | | | | | | |
| T16-18 | 103 | GEKGRQGNPG | 156 | 173 | 1852.9503 | 1852.9458 | 0.004281 | 6.76 | 1,738.51 |
| | | LQGTEGLR | | | | | | | |
| T16-20 | 104 | GEKGRQGNPG | 156 | 182 | 2795.3911 | 2795.5877 | −0.0033 | 5.51 | 8,967.10 |
| | | LQGTEGLRGE | | | | | | | |
| | | QGEKGEK | | | | | | | |
| T17 | | GR | 159 | 160 | 232.1484 | Not detected | | | |
| T17-18-clipL | 105 | GRQGNPGLQG | 159 | 172 | 1382.6902 | 1382.6887 | −0.001582 | 7.58 | 12,758.85 |
| | | TEGL | | | | | | | |
| T17-18 | 106 | GRQGNPGLQG | 159 | 173 | 1538.7914 | 1538.7919 | 0.000687 | 6.31 | 25,249.46 |
| | | TEGLR | | | | | | | |
| T17-19 | 107 | GRQGNPGLQG | 159 | 179 | 2167.073 | 2167.0701 | −0.002904 | 5.83 | 106,768.80 |
| | | TEGLRGEQGE | | | | | | | |
| | | K | | | | | | | |
| T17-20 | 108 | GRQGNPGLQG | 159 | 182 | 2481.2319 | 2481.2322 | 0.000921 | 5.59 | 36,064.79 |
| | | TEGLRGEQGE | | | | | | | |
| | | KGEK | | | | | | | |
| T17-21-clipD | 109 | GRQGNPGLQG | 159 | 184 | 2653.2865 | 2653.2291 | −0.001494 | 6.66 | 10,397.58 |
| | | TEGLRGEQGE | | | | | | | |
| | | KGEKGD | | | | | | | |
| T18 | 110 | QGNPGLQGTE | 161 | 175 | 1326.676 | Not detected | | | |
| | | GLR | | | | | | | |
| T18-19 | 111 | QGNPGLQGTE | 161 | 179 | 1936.9238 | 1936.9227 | −0.001056 | 7.68 | 14,110.98 |
| | | GLRGEQGEK | | | | | | | |
| T18-20 | 112 | QGNPGLQGTE | 161 | 182 | 2268.1094 | 2268.1101 | 0.000698 | 5.98 | 60,817.64 |
| | | GLRGEQGEKG | | | | | | | |
| | | EK | | | | | | | |
| T18-21-clipD | 113 | QGNPGLQGTE | 161 | 184 | 2440.158 | 2440.1602 | 0.002185 | 6.14 | 17,499.44 |
| | | GLRGEQGEKG | | | | | | | |
| | | EKGD | | | | | | | |
| T18-21-clipR | 114 | RGEQGEKGEK | 173 | 188 | 1711.8601 | 1711.8606 | 9.05E−05 | 5.06 | 7,889.07 |
| | | GDPGIR | | | | | | | |
| T19 | 115 | GEQGEK | 174 | 179 | 647.2995 | Not detected | | | |
| T19-21 | 116 | GEQGEKGEKG | 174 | 188 | 1555.759 | 1555.7548 | −0.00416 | 5.3 | 24,352.07 |
| | | DPGIR | | | | | | | |
| T20 | | GEK | 180 | 182 | 333.1768 | Not detected | | | |
| T20-21 | 117 | GEKGDPGIR | 180 | 188 | 927.47742 | 927.47589 | −0.001604 | 5.25 | 156,253.20 |

| | | | | | | |
|---|---|---|---|---|---|---|
| T21 | 118 | GDPGIR | 183 | 188 | 614.3256 | Not detected |

Analysis was also performed to evaluate any amino acid or peptide modifications present in the produced polypeptide of SEQ ID NO: 2 (Table 6). In a few instances, additional confirmatory analyses were performed to differential methionine oxidation from the presence of hydroxyproline residues. For example, based upon the fragmentation results from MS/MS scans, the tryptic peptide T1 (sequence DTGFPGMPGR (SEQ ID NO: 41)) was shown to contain a methionine oxidation rather than a proline hydroxylation. Based on such results, it was conclusively determined that tryptic peptide 1 (T1) has oxidation at methionine position 7 and no evidence of hydroxyproline at position 5 or 8. Similarly, where there is another methionine in position 83 in tryptic peptide 9 (T9), there were no detectable levels of methionine oxidation, hydroxyproline in positions 77, 85, 92, 95, and 97, or hydroxylysine at position 98 of the polypeptide. Accordingly, the truncated collagen polypeptides of the present disclosure also differ from naturally occurring collagen polypeptides in their lack of hydroxyproline residues.

TABLE 6
Analysis of amino acid and peptide modifications of the polypeptide of SEQ ID NO: 2.

| | | | | Relative |
|---|---|---|---|---|
| | | | Intensity | Instensity |
| Modifications | Label | Sequence | (counts) | (%) |
| Oxidation Met, Missed | T1 | Oxidation (M) | 84,835.78 | 3.29 |
| Cleavages, N and | | Clippped (T) | 4,768.16 | 0.18 |
| C terminal Clips | T1-2 | Missed Cleavage, Clippped (D) | 126,314.00 | 4.90 |
| | | Missed Cleavage | 577,210.30 | 22.38 |
| | T1-3 | 2 Missed Cleavages | 1,090,023.00 | 42.26 |
| | | 2 Missed Cleavages, Clipped (G) | 228,217.00 | 8.85 |
| | | 2 Missed Cleavages, Clipped (P1) | 436,082.90 | 16.91 |
| | | 2 Missed Cleavages, Clipped (P2) | 25,095.66 | 0.97 |
| | T1-4 | 3 Missed Cleavages | 6,936.58 | 0.27 |
| Missed Cleavages, | T4 | Unmodified | 233,635.20 | 23.83 |
| N and C Terminal | T4-5 | Missed Cleavgae, Clipped (E) | 3,738.19 | 0.38 |
| Clips | | Missed Cleavage | 112,201.00 | 11.45 |
| | T4-6 | 2 Missed Cleavages | 441,091.10 | 44.99 |
| | | 2 Missed Cleavages, Clipped (P) | 14,717.96 | 1.50 |
| | T4-7 | 3 Missed Cleavages | 114,096.20 | 11.64 |
| | | 3 Missed Cleavages, Clipped (G) | 60,853.30 | 6.21 |
| Missed Cleavages, | T5 | Unmodified | 4,628.28 | 1.63 |
| N Terminal Clip | T5-6 | Missed Cleavage | 71,435.91 | 25.11 |
| | | Missed Cleavage, Clipped (R) | 3,424.51 | 1.20 |
| | T6 | Unmodified | 177,768.80 | 62.48 |
| | T5-7 | 2 Missed Cleavages | 14,067.67 | 4.94 |
| | T6-7 | Missed Cleavage | 13,208.12 | 4.64 |
| Missed Clevages, N | T6-8 | 2 Missed Cleavages | 24,234.60 | 26.78 |
| Terminal Clips, Methyl Ile, | T6-9 | 3 Missed Cleavages | 9,805.94 | 10.83 |
| Dehydrated Gln | T7-8 | Missed Cleavage, Clipped (P) | 4,871.83 | 5.38 |
| | | Missed Cleavage, Methyl (I) | 3,103.51 | 3.43 |
| | | Missed Cleavage, Clipped (K) | 11,881.92 | 13.13 |
| | T7-9 | 2 Missed Cleavages, Clipped (P) | 1,757.15 | 1.94 |
| | T8 | Dehydrated (E) | 6,818.71 | 7.53 |
| | | Clipped (I) | 28,037.06 | 30.98 |
| Cation K, Missed | T9 | Cation K | 48,341.35 | 19.07 |
| Cleavgaes, N Terminal | | Clipped (T) | 5,679.79 | 2.24 |
| Clips | | Clipped (G) | 1,873.69 | 0.74 |
| | | Clipped (S) | 904.06 | 0.36 |
| | T9-10 | Missed Cleavage | 99,126.63 | 39.11 |
| | | Missed Cleavage, Clipped (D) | 10,231.43 | 4.04 |
| | T10-11 | Missed Cleavage, Clipped (K) | 5,921.06 | 2.34 |
| | T8-11 | 3 Missed Cleavages | 81,370.18 | 32.11 |
| Missed Cleavages | T11 | Unmodified | 40,875.16 | 72.47 |
| | T11-13 | 2 Missed Cleavages | 15,528.34 | 27.53 |
| Missed Cleavages, | T12 | Unmodified | 145,576.30 | 51.93 |
| N Terminal Clips | T12-13 | Missed Cleavage | 45,482.75 | 16.23 |
| | T12-14 | 2 Missed Cleavages, Clipped (P4) | 18,260.02 | 6.51 |
| | | 2 Missed Cleavages, Clipped (P3) | 16,118.65 | 5.75 |
| | | 2 Missed Cleavages, Clipped (P2) | 16,247.45 | 5.80 |
| | | 2 Missed Cleavages, | 24,994.64 | 8.92 |
| | T12-15 | 3 Missed Cleavages, Clipped (P) | 13,644.56 | 4.8 |
| Missed Cleavages | T13-14 | Missed Cleavage | 66,051.58 | 36.91 |
| | T13-16 | 3 Missed Cleavages | 2,494.46 | 1.39 |
| | T13-17 | 4 Missed Cleavages | 2,147.75 | 1.20 |
| | T14 | Unmodified | 101,400.00 | 56.67 |
| | T14-15 | Missed Cleavages | 2,745.49 | 1.53 |
| | T14-17 | 3 Missed Cleavages | 2,134.92 | 1.19 |
| | T14-16 | 2 Missed Cleavages | 1,968.24 | 1.10 |

### Example 3. A Non-Naturally Occurring Polypeptide Having an Amino Acid Sequence of SEQ ID NO: 8 is Non-Toxic to Human Fibroblasts and Keratinocytes

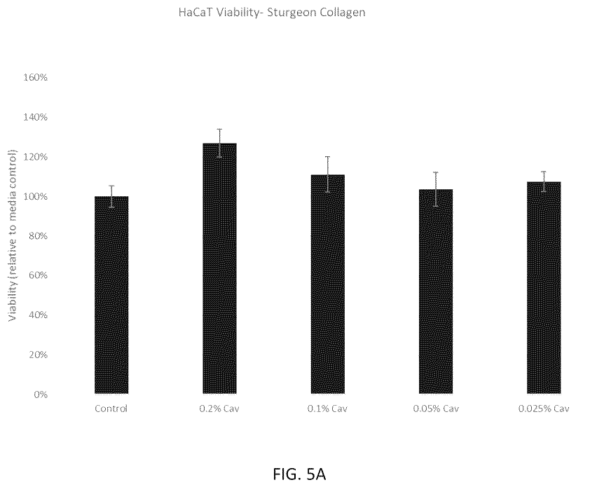

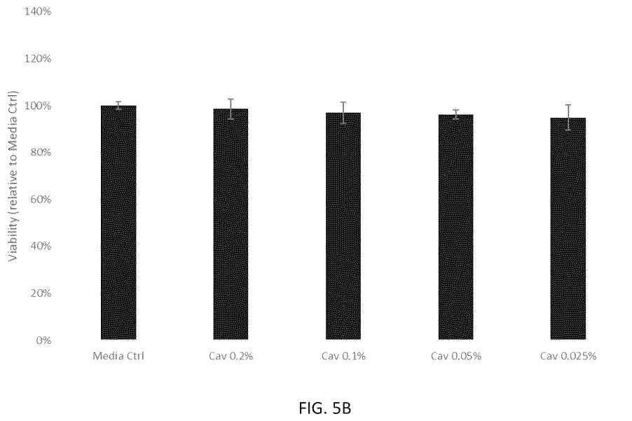

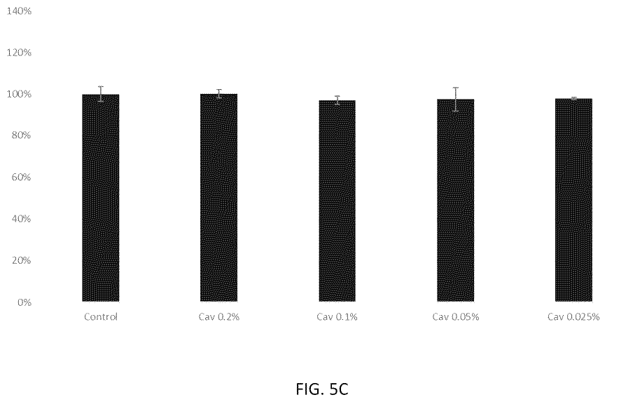

Human primary fibroblasts, HaCaT cells, and human primary keratinocytes treated with a non-naturally occurring polypeptide having an amino acid sequence of SEQ ID NO: 8 (indicated as “Cav” in FIGS. 5A-5C) in vitro showed no sign of toxicity, as shown in FIGS. 5A-5C, indicating the product is safe as a topical ingredient at the dosages tested.

### Protocol:

The cells were seeded at confluency in a 96-well plate. 24 hours later, the media was changed to low serum media (to avoid any effects due to serum). The cells were treated with a non-naturally occurring polypeptide having the amino acid sequence of SEQ ID NO: 8 in the same low serum media for 24 hours. Post treatment with the polypeptide, the supernatants were saved, and cells were incubated with MTT dye for 60 minutes at 37° C. MTT is metabolized to formazan salts by viable cells. These salts were dissolved using isopropanol and the color produced was quantified using a cell plate reader.

### Toxicology Analyses of a Formulation Comprising a Non-Naturally Occurring Polypeptide of the Disclosure

A variety of toxicology assays were performed in vitro to screen for any potential negative impact of formulations containing a non-naturally polypeptide having an amino acid sequence of SEQ ID NO: 8.

### 1) Bacterial Reverse Mutation Assay

The polypeptide was evaluated for the ability to induce a mutagenic response in four different strains of Salmonella typhimurium and an E. coli strain. Samples were screened at different dose levels by plating them with the tester strains both with and without Arocolor™ 1254 induced rat liver microsomes (S9). Samples are considered mutagenic if they cause an increase in revertant colonies above the spontaneous background level. The assay is known in the art and is performed compliant with OECD 4714 Guideline for Testing of Chemicals: Bacterial Reverse Mutation Assay.

A powder of a polypeptide having an amino acid sequence of SEQ ID NO: 8 was prepared in sterile deionized water at 5 concentrations: 5 mg/plate, 1 mg/plate, 0.5 mg/plate, 0.1 mg/plate, and 0.05 mg/plate. Testing was done with the appropriate solvent control and positive controls were plated with overnight cultures on selective minimal agar in the presence and absence of Aroclor-induced rate liver S9. All were plated in triplicate.

Results showed that test strains were sensitive to the positive control mutagens and showed the appropriate mutagenic response. The spontaneous reversion rate indicated that the strains were sensitive to the detection of potentially genotoxic agents. The formulations were not found to be cytotoxic to the test systems. The metabolic activation using the S9 activation mixture showed an active microsomal preparation. The formulations showed no detectable genotoxic activity at any concentration either in the presence or absence of S9 enzyme activation.

### 2) EpiDerm™ Skin Model In Vitro Toxicity Testing System

Sturgeon collagen was evaluated for irritancy potential utilizing the MatTek Corporation EpiDerm™ in vitro toxicity testing system as is known in the art. Briefly, normal, human-derived epidermal keratinocytes (NHEK) which have been cultured to form a multilayered, highly differentiated model of the human epidermis were tested with substances and evaluated for damage to mitochondrial enzyme succinate dehydrogenase, as monitored by a color reaction. The enzyme converts a water-soluble, yellow MTT to a purple, insoluble product, and the amount of MTT converted is proportional to the number of viable cells. Triton X-100 (1%) was used as a positive control. Results are depicted in Table 7 (GEL-CAV-A indicating the treatment with polypeptide having an amino acid sequence of SEQ ID NO: 8).

TABLE 7
Results of in vitro toxicity testing

| Article | | Percent | Percent |
|---|---|---|---|
| (% & Exposure) | System | Viability | Inhibition |
| GEL-CAV-A EpiDerm; Lot | | | |
| Number: PP6-CAV-20-342 | | | |
| (100%-20 hrs.) | EpiDerm | 80 | 20 |
| (100%-4.5 hr.) | EpiDerm | 98 | 2 |
| (100%-1 hr.) | EpiDerm | 101 | −1 |
| Triton X-100 | | | |
| (1%-20 brs.) | EpiDerm | 5 | 95 |
| (1%-4.5 hr.) | EpiDerm | 94 | 6 |
| (1%-1 hr.) | EpiDerm | 99 | 1 |

The time at which viability would be 50%, ET-50, for the polypeptide was determined to be greater than 24 hours, and the positive control at 9.4 hours. Standard ranges are shown in Table 8, according to the manufacturer.

TABLE 8
Standard ranges for EpiDerm ™ Skin Model in vitro Toxicity Testing System

| ET-50 (hrs) | Expected In vivo Irritancy | Example |
|---|---|---|
| <0.5 | Severe, probably corrosive | Conc. Nitric Acid |
| 0.5-4 | Moderate | 1% Sodium Dodecyl Sulfate |
| 4-12 | Moderate to Mild | 1% Triton X-100 |
| 12-24 | Very Mild | Baby Shampoo |
| 24 | Non-irritating | 10% Tween 20 |

Accordingly, the polypeptide has an expected in vivo dermal irritancy potential in the non-irritating range.

### 3) EpiOcular™ Tissue Model In Vitro Toxicity Testing System

The polypeptide was evaluated for irritancy potential utilizing the MatTek Corporation EpiOcular™ in vitro toxicity testing system as is known in the art. Briefly, normal, human-derived epidermal keratinocytes which have been cultured to form a stratified, squamous epithelium similar to that found in the cornea were tested with substances and evaluated for damage to mitochondrial enzyme succinate dehydrogenase, as monitored by a color reaction. The enzyme converts a water-soluble, yellow MTT to a purple, insoluble product, and the amount of MTT converted is proportional to the number of viable cells. Triton X-100 (0.3%) is used as a positive control. Results are depicted in Table 9 (GEL-CAV-A indicating the treatment with polypeptide having an amino acid sequence of SEQ ID NO: 8).

TABLE 9
Results from in vitro toxicity testing

| Article | | Percent | Percent |
|---|---|---|---|
| (% & Exposure) | System | Viability | Inhibition |
| GEL-CAV-A EpiOcular; | | | |
| Lot Number: PP6-CAV-20-342 | | | |
| (20%-4 hrs.) | BpiOcular | 101 | −1 |
| (20%-1 hr.) | EpiOcular | 98 | 2 |
| (20%-20 mins.) | EpiOcular | 111 | −11 |
| Triton X-100 | | | |
| (0.3%-1 hr.) | EpiOcular | 11 | 89 |
| (0.3%-20 mins.) | EpiOcular | 49 | 51 |
| (0.3%-5 mins.) | EpiOcular | 82 | 18 |

The time at which viability would be 50%, ET-50, was determined and then used to estimate the rabbit Draize eye score: Draize=−4.74+101.7/(ET−50)\hat{ }0.5 as is known in the art. The polypeptide was found to have an ET-50 greater than 256 minutes, and an estimated Draize ocular irritation score of 0 (the positive control at 19.6 minutes/Draize 18.2). Standard ranges are shown in Table 10 according to the manufacturer.

TABLE 10
Standard ranges for EpiOcular ™ Tissue Model in vitro toxicity testing system

| Draize Score | Irritancy Classification | Example | EpiOcular ET-50 (min) |
|---|---|---|---|
| 0-15 | Non-irritating, Minimal | PEG-75 Lanolin, Tween 20 | >256-26.5 |
| 15.1-25 | Mild | 3% Sodium Dodecyl Sulfate | <26.5-11.7 |
| 25.1-50 | Moderate | 5% Triton X-100 | <11.7-3.45 |
| 50.1-110 | Severe, Extreme | 5% Benzalkonium Chloride | <3.45 |

Accordingly, the polypeptide has a non-irritating irritancy classification.

### 4) Repeated Insult Patch Study

The polypeptide was evaluated to determine its ability to sensitize the skin of volunteer subjects with normal skin using an occlusive repeated insult patch study as is known in the art. Briefly, repeated insult patch evaluation is a modified predictive patch study that can detect weak sensitizers that require multiple applications to induce a cell-mediated (Type IV) immune response sufficient to cause an allergic reaction. Irritant reactions may also be detected using this evaluation method, although this is not the primary purpose of this procedure. Sodium laurylsulfate, 0.2% aqueous solution served as a positive control.

Ninety-five (95) subjects completed the study. Under the conditions employed in this study, there was no evidence of sensitization to the polypeptide formulation.

### Example 4. A Non-Naturally Occurring Polypeptide Having an Amino Acid Sequence of SEQ ID NO: 8 Promotes Keratinocyte Growth and Regeneration

Healthy skin is primarily composed of collagen types I and III, hyaluronans, fibronectin and elastin, and a basal lamina that includes other proteins such as laminins and collagen IV. Fibroblasts are the major cell type that produces these structural proteins, including collagen. Collectively the proteins are known as extra cellular matrix (ECM) and they support the skin's structure. Fibroblast output of collagen decreases with age, so fibroblasts are a primary target for the activity of cosmetics to try to rescue skin aging.

Keratinocytes are the major cell type forming the epidermis, or outer layers of the skin. HaCaT cells are an immortal keratinocyte cell line derived from adult skin. Both cell types were used to demonstrate the benefits of a non-naturally occurring polypeptide having an amino acid sequence of SEQ ID NO: 8 on skin (indicated as “Cav” in figures). These cells have a high turn-over and receive the brunt of everyday pollution and radiation. They are negatively affected by the environments they are subjected to, which leads to increased inflammation and damage to our natural skin barrier. Hallmarks used to assess keratinocyte health include inflammatory markers, cell turnover, and DNA integrity.

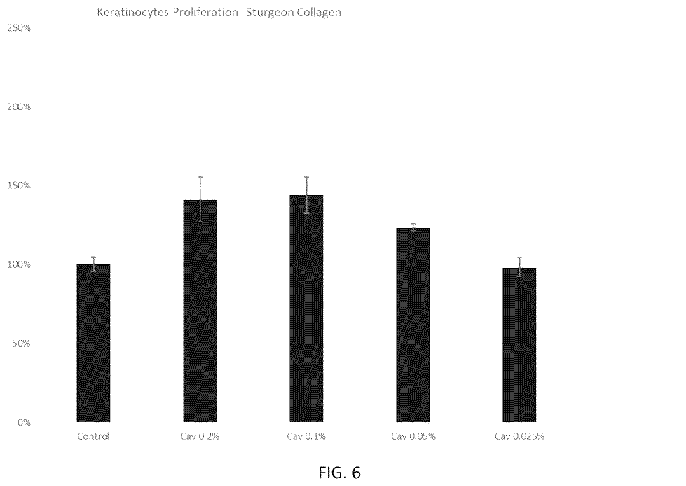

Keratinocytes treated with a non-naturally occurring polypeptide having an amino acid sequence of SEQ ID NO: 8 showed a dose-dependent increase in keratinocyte growth and regeneration. Similar results were seen immortal HaCaT keratinocytes. As shown in FIG. 6 (human primary keratinocytes), the polypeptide (indicated as “Cav” in FIG. 6) demonstrated a dose-dependent stimulation of cellular growth and regeneration in keratinocytes, with a 40% increase in cell numbers at 0.2% (w/w) and 0.1% (w/w) treatment, when compared with control cells.

### Example 5. A Non-Naturally Occurring Polypeptide Having an Amino Acid Sequence of SEQ ID NO: 8 Stimulates Collagen Production and Upregulation of Genes Involved in Cell Proliferation, Migration, and Adhesion

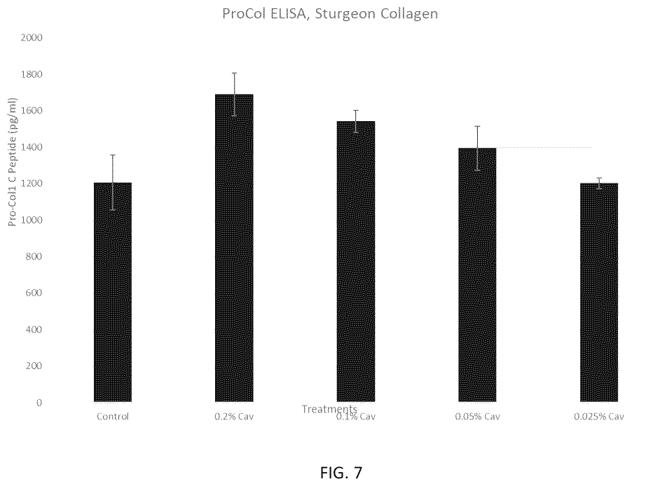

A non-naturally occurring polypeptide having an amino acid sequence of SEQ ID NO: 8 (indicated as “Cav” in FIG. 7) stimulated production of collagen I production by in vitro fibroblasts as shown in FIG. 7.

ELISA Protocol: Primary human fibroblasts were cultured in standard media DMEM/F12+10% FBS. Supernatants were used to determine the level of collagen type I present. The kit used was Takara Procollagen type I C-peptide detection ELISA kit. Manufacturer's protocol was followed to measure the quantity of collagen type I in the supernatants.

In a second method of analysis, microarray data reporting the levels of RNA for a variety of human collagens showed a 2.5-3-fold increase in expression of these collagens in fibroblasts treated with the polypeptide. Table 11 depicts the microarray data.

Microarray RNA analysis Protocol: The cells were seeded at confluency in 6-well plates. 24 hours later the media was changed to low serum media. The cells were treated with 0.05% (w/w) of the polypeptide and control. The QIAGEN RNeasy kit was used to extract the RNA and the extracted RNA for analysis.

TABLE 11
Microarray data

| ID | 7 Avg | 8 Avg | Fold | | FDR | Gene | | |
|---|---|---|---|---|---|---|---|---|
| count: 10 | (log2) | (log2) | Chan . . . | P-val | P-val | Symbol | Description | Group |
| TC1700011088.h . . . | 9.08 | 7.42 | 3.16 | 4.15E−06 | 0.0011 | COL1A1 | collagen, type I, alpha 1 | Multiple . . . |
| TC0700008358.h . . . | 15.3 | 13.86 | 2.71 | 7.01E−06 | 0.0011 | COL1A2 | collagen, type I, alpha 2 | Multiple . . . |
| TC0200016193.h . . . | 14.16 | 12.26 | 3.73 | 7.69E−06 | 0.0011 | COL6A3 | collagen, type VI, alpha 3 | Multiple . . . |
| TC0600012314.h . . . | 11.16 | 9.46 | 3.26 | 8.77E−06 | 0.0011 | COL12A1 | collagen, type XII, alpha 1 | Multiple . . . |
| TC2100007446.h . . . | 13.82 | 12.32 | 2.82 | 1.01E−05 | 0.0012 | COL6A1 | collagen, type VI, alpha 1 | Multiple . . . |
| TC0200010239.h . . . | 10.42 | 8.93 | 2.81 | 2.99E−05 | 0.0015 | COL3A1; . . . | collagen, type III, alpha 1; . . . | Multiple . . . |
| TC2100007451.h . . . | 9.11 | 7.91 | 2.3 | 9.13E−05 | 0.0022 | COL6A2 | collagen, type VI, alpha 2 | Multiple . . . |
| TC0500011163.h . . . | 10.42 | 9.22 | 2.29 | 0.0001 | 0.0023 | COL4A3BP | collagen, type IV, alpha 3 . . . | Multiple . . . |
| TC0900009127.h . . . | 8.32 | 6.39 | 3.8 | 0.0004 | 0.0052 | COL5A1 | collagen, type V, alpha 1 | Multiple . . . |
| TC1300008010h . . . | 5.98 | 4.98 | 2 | 0.0022 | 0.0155 | COL4A2 | collagen, type IV, alpha 2 | Multiple . . . |

In addition to the upregulation of collagens, the polypeptide was found to increase the levels of RNA for a variety of genes involved in several pathways responsible for proliferation, migration, and adhesion.

### Upregulated Pathways:

### VEGFA-VEGFR2 Signaling Pathway

Number of upregulated genes: 74
Number of down regulated genes: 12
Significance: 7.74

TABLE 12
Exemplary upregulated genes in the VEGFA-VEGFR2 signaling pathway

| Symbol | Fold change | P-value | Description |
|---|---|---|---|
| MYOC1 | 3.25 | 9.25E−05 | Myosin IC |
| FLII | 4.08 | 1.06E−05 | Flightless I actin |
| | | | binding protein |
| ROCK1 | 3.72 | 8.22E−07 | Rho-associated, coiled- |
| | | | coil containing protein |
| | | | kinase 1 |
| ROCK2 | 5.03 | 6.31E−05 | Rho-associated, coiled- |
| | | | coil containing protein |
| | | | kinase 2 |
| CLTC | 2.7 | 7.06E−06 | Clathrin, heavy chain |
| | | | (Hc) |
| LIMK 1 | 4.24 | 2.04E−06 | Serine/threonine |
| | | | Kinase |
| EGR1 | 3.28 | 2.27E−05 | Early growth response |

### Focal Adhesion Pathway

No of upregulated genes: 53
No of down regulated genes: 0
Significance: 9.93

TABLE 13
Exemplary upregulated genes in the Focal Adhesion Pathway

| Symbol | Fold change | P-value | Description |
|---|---|---|---|
| ITGA3 | 3.45 | 5.71E−07 | Integrin alpha 3 |
| TNC | 3.13 | 5.53E−05 | Tenascin |
| LAMC1 | 3.15 | 1.15E−05 | Laminin, gamma 1 |
| FLNA | 6.5 | 1.47E−07 | Actin-binding protein |
| | | | that regulates |
| | | | reorganization of |
| | | | actin cytoskeleton |
| TLN1 | 3.8 | 0.0001 | Cytoskeletal protein |
| ZYX | 3.8 | 6.81E−06 | Zinc-binding |
| | | | phosphoprotein and |
| | | | is found in mature |
| | | | adhesions |
| DIAPH1 | 3.78 | 8.79E−05 | Regulates cell |
| | | | morphology and |
| | | | cytoskeletal |
| | | | reorganization |

### Endothelin Pathway

No of upregulated genes: 48
No of down regulated genes: 4
Significance: 3.57

TABLE 14
Exemplary upregulated genes in the Endothelin Pathway

| Symbol | Fold change | P-value | Description |
|---|---|---|---|
| TRIOBP | 3.91 | 4.55E−06 | TRIO and F-actin |
| | | | binding protein; |
| | | | nucleolar protein 12 |
| WNK1 | 3.11 | 9.10E−05 | Serine/threonine |
| | | | protein kinase |
| MMP2 | 3.08 | 1.13E−06 | Matrix |
| | | | metallopeptidase 2 |
| VCAN | 3.31 | 3.10E−06 | Versican |
| ACTA2 | 2.52 | 0.0002 | Actin, alpha 2 |
| GNA12 | 3.88 | 5.99E−06 | Guanine nucleotide |
| | | | binding protein (G- |
| | | | protein) alpha 12 |
| EGR1 | 3.28 | 2.27E−05 | Early growth response |

### EGF/EGER Signaling Pathway

No of upregulated genes: 32
No of down regulated genes: 4
Significance: 4.83

TABLE 15
Exemplary upregulated genes in the EGF/EGFR Signaling Pathway

| Symbol | Fold change | P-value | Description |
|---|---|---|---|
| ATXN2 | 3.6 | 2.48E−05 | Interact with |
| | | | endoplasmic |
| | | | reticulum |
| JAK1 | 3.15 | 1.88E−05 | Involved in cell |
| | | | growth, survival, |
| | | | development, and |
| | | | differentiation |
| RPS6KA2 | 3.12 | 1.96E−06 | Ribosomal protein |
| ROCK1 | 3.72 | 8.22E−07 | Rho-associated, |
| | | | coiled-coil |
| | | | containing protein |
| | | | kinase 1 |
| SHC1 | 2.83 | 2.57E−06 | Regulation of |
| | | | apoptosis |
| IQGAP1 | 2.65 | 1.94E−05 | Scaffold protein, |
| | | | help in modulating |
| | | | several cellular |
| | | | activities. |
| PLCG1 | 2.59 | 0.0011 | Ribosomal protein |

### TGF-Beta Signaling Pathway

No of upregulated genes: 33
No of down regulated genes: 3
Significance: 6.92

TABLE 16
Exemplary upregulated genes in the TGF-beta signaling pathway

| Symbol | Fold change | P-value | Description |
|---|---|---|---|
| SMURF1 | 2.68 | 4.20E−06 | SMAD specific E3 |
| | | | ubiquitin protein |
| | | | kinase 1 |
| SPTBN1 | 3.41 | 4.83E−06 | Responsible for |
| | | | cellular shape, |
| | | | protection of |
| | | | membranes against |
| | | | stress |
| PAK2 | 2.71 | 1.68E−05 | Stimulates cell |
| | | | survival and cell |
| | | | growth |
| ROCK1 | 3.72 | 8.22E−07 | Rho-associated, |
| | | | coiled-coil |
| | | | containing protein |
| | | | kinase 1 |
| SHC1 | 2.83 | 2.57E−06 | Regulation of |
| | | | apoptosis |
| TGFBR3 | 2.6 | 2.05E−05 | Transforming |
| | | | growth factor beta |
| | | | receptor III |
| TGFBR1 | 2.6 | 0.0062 | Transforming |
| | | | growth factor beta |
| | | | receptor I |

### Example 6. A Non-Naturally Occurring Polypeptide Having an Amino Acid Sequence of SEQ ID NO: 8 Promotes Wound Healing Activity

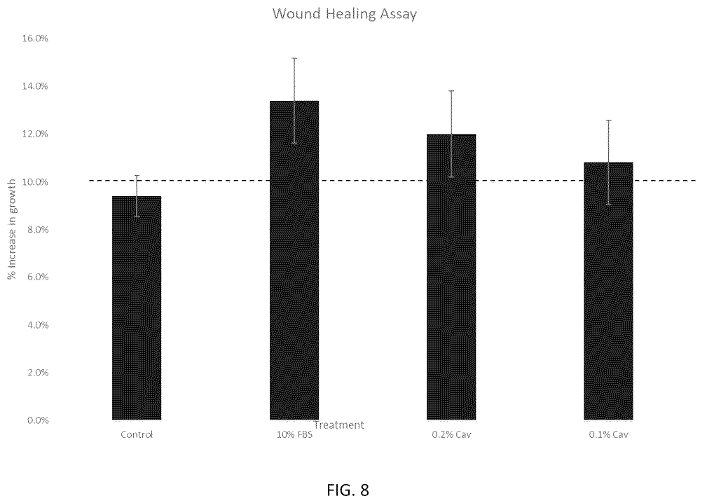

Wound healing is a dynamic process that includes a sequence of events, including cell proliferation and migration. Fibroblast migration and proliferation play a crucial role in wound closure by secreting various chemicals, including collagen and other matrix proteins. Treatment of in vitro human dermal fibroblasts with a non-naturally occurring polypeptide having an amino acid sequence of SEQ ID NO: 8 showed wound healing activity in an in vitro wound-healing model as shown in FIG. 8, as cells proliferated and closed a gap induced by scratching a confluent layer of fibroblasts. Additionally, microarray data was consistent with the polypeptide having a wound healing benefit. The data also showed upregulation of genes involved in several pathways responsible for cell proliferation, migration, and adhesion.

Protocol: The cells were seeded at confluency in 24 well plate. 24 hours later the media was changed to low serum media and the cells were starved for 6-8 hours. Post starvation, the wells containing cells were scratched and treated. Images were taken at this time (time 0 hours) and after 24 hours. Images were analyzed using Image J software.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

1. A method of treating a wound and/or a proliferative disorder in skin of a subject, the method comprising:
administering to the skin of the subject a therapeutically effective amount of a polypeptide comprising an amino acid sequence having at least 80% sequence identity to SEQ ID NO: 32, or a functional truncate of SEQ ID NO: 32;
thereby treating the wound and/or the proliferative disorder.

2. The method of claim 1,
wherein the proliferative disorder of the skin is characterized by abnormal proliferation, migration, and/or adhesion of skin cells (e.g., fibroblasts, keratinocytes).

3. The method of claim 1, wherein the wound exhibits impaired wound healing.

4. The method of claim 1, wherein the method comprises administering the polypeptide to the wound or to skin adjacent to the wound.

5. The method of claim 2, wherein the abnormal proliferation, migration, and/or adhesion is decreased or reduced proliferation, migration, and/or adhesion.

6. The method of claim 2, wherein the skin condition is epidermal thinning, epidermal atrophy, dermal atrophy, epidermal degeneration, acantholysis, pemphigus foliaceus, pemphigus vulgaris, acantholytic dyskeratosis, Darier disease, Hailey-Hailey disease, Grover disease, lichen sclerosus, hyalinisation of collagen, or a combination thereof.

7. The method of claim 1, wherein the polypeptide comprises an amino acid sequence having at least 85% sequence identity to SEQ ID NO: 32, or a functional truncate of SEQ ID NO: 32.

8. The method of claim 1, wherein the polypeptide comprises or consists of an amino acid sequence having at least 90% sequence identity to SEQ ID NO: 32, or a functional truncate of SEQ ID NO: 32.

9. The method of claim 1, wherein the polypeptide comprises an amino acid sequence having at least 95% sequence identity to SEQ ID NO: 32, or a functional truncate of SEQ ID NO: 32.

10-11. (canceled)

12. The method of claim 1, wherein the truncate of SEQ ID NO: 32 comprises an N-terminal truncation, a C-terminal truncation, or both, relative to SEQ ID NO: 32.

13. The method of claim 12, wherein the N-terminal truncation comprises a truncation of 50 amino acids to 700 amino acids relative to SEQ ID NO: 32.

14. The method of claim 12, wherein the C-terminal truncation comprises a truncation of 50 amino acids to 550 amino acids relative to SEQ ID NO: 32.

15. The method of claim 1, wherein the polypeptide comprises the amino acid sequence of SEQ ID NO: 8.

16-17. (canceled)

18. The method of claim 1, wherein the polypeptide is 50 amino acids to 250 amino acids in length.

19. The method of claim 1, wherein the polypeptide does not comprise one or more of: a laminin G domain, a Von Willebrand factor type A (vWA) domain, and/or a fibrillar collagen C-terminal domain.

20-21. (canceled)

22. The method of claim 1, wherein the polypeptide does not form a stable triple helix structure of a naturally occurring collagen.

23-24. (canceled)

25. The method of claim 1, wherein fewer than 10% of prolines present in the polypeptide are hydroxylated.

26-43. (canceled)

44. A pharmaceutical composition comprising the polypeptide of claim 1; and a pharmaceutically acceptable excipient.

45-66. (canceled)

67. The pharmaceutical composition of claim 44, wherein the pharmaceutically acceptable excipient is selected from the group consisting of: an antiadherent, a binder, a coating, a color, a disintegrant, a flavor, a glidant, a lubricant, a preservative, a sorbent, a vehicle, and any combination thereof.

68. (canceled)

69. The pharmaceutical composition of claim 44, wherein the pharmaceutical composition is formulated as a gel, a cream, a lotion, an oil, a foam, an ointment, a serum, and any combination thereof.


SEQUENCE LISTING (118 sequences)

SEQ ID NO: 1 - DNA, 564 bp, Gallus gallus
  source 1..564: mol_type = genomic DNA
gataccggttttccgggtatgcctggtcgtagcggtgatccgggtcgtagcggtaaagatggtctgcctggtagcccgggttttaaaggtgaagttggtcagccaggtagccctggtctggaaggtcatcgtggtgaaccgggtattccaggtattccgggtaatcagggtgcaaaaggtcagaaaggcgaaattggtcctccgggtctgccaggtgccaaaggttctccgggtgaaaccggtctgatgggtcctgaaggtagctttggcctgcctggtgcaccgggtccgaaaggtgacaaaggtgaacctggtctgcagggtaaaccgggtagcagcggtgcaaaaggcgaaccaggtggtccgggtgctccgggtgaaccaggctatccgggtattcctggtactcagggtattaaaggcgataaaggtagccagggtgaaagcggtattcagggtcgtaagggtgaaaaaggccgtcagggtaatccaggcctgcagggcaccgaaggtctgcgtggcgaacagggcgaaaaaggtgagaagggtgacccaggcattcgt

SEQ ID NO: 2 - protein, 188 aa, Gallus gallus
DTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIR

SEQ ID NO: 3 - DNA, 564 bp, Gallus gallus
  source 1..564: mol_type = genomic DNA
gatactggtttcccggggatgcctgggcgctcaggtgatccggggcgtagtggaaaagacggtctgccggggtccccgggctttaagggtgaggtgggtcagcccggtagtccaggtttagaaggtcaccgcggagagcccgggattccaggcattcctggcaaccagggtgccaagggacagaaaggcgaaattggtccgcccggcctaccgggcgcgaaaggttctcctggtgaaaccggtctcatgggtccggaaggtagcttcggcctgcccggcgcacctggtccgaagggcgataagggggagcctgggctgcaaggtaaaccgggtagttctggcgccaaaggtgaacccggcggtcccggtgcgccaggggaaccaggttatcctggtattcctggaacccaaggaattaaaggtgacaaaggctcacagggcgaaagtggtatacagggtcgcaagggcgaaaaaggacgtcagggcaatccaggcctgcagggtactgaaggcctgcgtggagaacagggtgagaaaggtgaaaaaggagatcctggtattcgc

SEQ ID NO: 4 - protein, 188 aa, Gallus gallus; identical to SEQ ID NO: 2

SEQ ID NO: 5 - DNA, 633 bp, Gallus gallus
  source 1..633: mol_type = genomic DNA
gatactggtttcccggggatgcctgggcgctcaggtgatccggggcgtagtggaaaagacggtctgccggggtccccgggctttaagggtgaggtgggtcagcccggtagtccaggtttagaaggtcaccgcggagagcccgggattccaggcattcctggcaaccagggtgccaagggacagaaaggcgaaattggtccgcccggcctaccgggcgcgaaaggttctcctggtgaaaccggtctcatgggtccggaaggtagcttcggcctgcccggcgcacctggtccgaagggcgataagggggagcctgggctgcaaggtaaaccgggtagttctggcgccaaaggtgaacccggcggtcccggtgcgccaggggaaccaggttatcctggtattcctggaacccaaggaattaaaggtgacaaaggctcacagggcgaaagtggtatacagggtcgcaagggcgaaaaaggacgtcagggcaatccaggcctgcagggtactgaaggcctgcgtggagaacagggtgagaaaggtgaaaaaggagatcctggtattcgcggcattaacggtcaaaagggtgaaagtgggatacaaggtcttgtcggtccgcccggagttagaggccag

SEQ ID NO: 6 - protein, 211 aa, Gallus gallus
DTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIRGINGQKGESGIQGLVGPPGVRGQ

SEQ ID NO: 7 - DNA, 596 bp, Acipenser schrenckii
  source 1..596: mol_type = genomic DNA
gtctgcagggtatgcctggtgaacgtggtgcaagcggtattgccggtgcaaaaggtgatcgtggtgatgttggtgaaaaaggtccggaaggtgccagcggtaaagatggtagccgtggtctgaccggtccgattggtccgcctggtccggcaggtccgaatggcgaaaaaggtgaaagcggtccgagcggtcctccgggtgcagcaggtactcgtggtgcaccgggtgatcgcggtgaaaatggtccaccgggtcctgccggttttgcaggtccgccaggtgcagatggtcagcctggtgccaaaggcgaacaaggcgaaggtggtcagaaaggtgatgcaggcgctccgggtccgcagggtccttctggtgcacctggtcctcagggtccgaccggtgtttctggtccgaaaggcgcacgtggtgcccagggtccacctggtgcgaccggttttcctggcgcagcaggtcgtgttggtcctccaggtcctaatggtaatccgggtccaagcggtcctgcaggtagcgcaggcaaagatggtcctaaaggtgtacgcggtgatgctggtcctcctggccgtgccggtgatgccggt

SEQ ID NO: 8 - protein, 199 aa, Acipenser schrenckii
GLQGMPGERGASGIAGAKGDRGDVGEKGPEGASGKDGSRGLTGPIGPPGPAGPNGEKGESGPSGPPGAAGTRGAPGDRGENGPPGPAGFAGPPGADGQPGAKGEQGEGGQKGDAGAPGPQGPSGAPGPQGPTGVSGPKGARGAQGPPGATGFPGAAGRVGPPGPNGNPGPSGPAGSAGKDGPKGVRGDAGPPGRAGDAG

SEQ ID NO: 9 - DNA, 57 bp, unidentified
  source 1..57: mol_type = unassigned DNA
atgaaaaagatttggctggcgctggctggtttagttttagcgtttagcgcatcggcg

SEQ ID NO: 10 - protein, 19 aa, unidentified
MKKIWLALAGLVLAFSASA

SEQ ID NO: 11 - DNA, 60 bp, unidentified
  source 1..60: mol_type = unassigned DNA
atgaaaaaaggtttcatgctgttcaccctcctcgctgcgttctctggtttcgcgcaggct

SEQ ID NO: 12 - protein, 20 aa, unidentified
MKKGFMLFTLLAAFSGFAQA

SEQ ID NO: 13 - DNA, 75 bp, unidentified
  source 1..75: mol_type = unassigned DNA
atgatgatcaccctgcgtaaactgccgctggctgttgctgttgctgctggtgttatgtctgctcaggctatggct

SEQ ID NO: 14 - protein, 25 aa, unidentified
MMITLRKLPLAVAVAAGVMSAQAMA

SEQ ID NO: 15 - DNA, 63 bp, unidentified
  source 1..63: mol_type = unassigned DNA
atgaaaaaaaccgctatcgctatcgctgttgctctggctggtttcgctaccgttgctcaggct

SEQ ID NO: 16 - protein, 21 aa, unidentified
MKKTAIAIAVALAGFATVAQA

SEQ ID NO: 17 - DNA, 63 bp, unidentified
  source 1..63: mol_type = unassigned DNA
atgaaagttaaagttctgtctctgctggttccggctctgctggttgctggtgctgctaacgct

SEQ ID NO: 18 - protein, 21 aa, unidentified
MKVKVLSLLVPALLVAGAANA

SEQ ID NO: 19 - DNA, 78 bp, unidentified
  source 1..78: mol_type = unassigned DNA
atgaaaaaaaacatcctgtctctgtctatggttgctctgtctctgtctctggctctgggttctgtttctgttaccgct

SEQ ID NO: 20 - protein, 26 aa, unidentified
MKKNILSLSMVALSLSLALGSVSVTA

SEQ ID NO: 21 - DNA, 69 bp, unidentified
  source 1..69: mol_type = unassigned DNA
atgctgaacccgaaagttgcttacatggtttggatgacctgcctgggtctgaccctgccgtctcaggct

SEQ ID NO: 22 - protein, 23 aa, unidentified
MLNPKVAYMVWMTCLGLTLPSQA

SEQ ID NO: 23 - DNA, 63 bp, unidentified
  source 1..63: mol_type = unassigned DNA
atgaaacaggctctgcgtgtagcgttcggtttcctgatactgtgggcttctgttctgcacgct

SEQ ID NO: 24 - protein, 21 aa, unidentified
MKQALRVAFGFLILWASVLHA

SEQ ID NO: 25 - DNA, 600 bp, synthetic construct
  source 1..600: mol_type = other DNA
ggtctgcagggtatgccgggtgaacgtggtgccagcggtattgcaggtgccaaaggtgatcgtggtgatgttggtgaaaaaggtccggaaggtgcaagcggtaaagatggtagccgtggtctgaccggtccgattggtccgccgggtccggccggtccgaatggtgaaaaaggtgaaagcggtccgagcggtccgccgggtgcagccggtacccgtggtgcaccgggtgatcgtggtgaaaatggtccgccgggtccggccggttttgcaggtccgccgggtgccgatggtcagccgggtgcaaaaggtgaacagggtgaaggtggtcagaaaggtgatgccggtgcaccgggtccgcagggtccgagcggtgccccgggtccgcagggtccgaccggtgttagcggtccgaaaggtgcacgtggtgcccagggtccgccgggtgcaaccggttttccgggtgccgcaggtcgtgttggtccgccgggtccgaatggtaatccgggtccgagcggtccggcaggtagcgccggtaaagatggtccgaaaggtgttcgtggtgatgcaggtccgccgggtcgtgccggtgatgcaggttaa

SEQ ID NO: 26 - DNA, 600 bp, synthetic construct
  source 1..600: mol_type = other DNA
ggcctgcaaggcatgccaggcgagcgcggcgcgtctggcatcgcgggcgcgaagggcgaccgcggcgacgtgggcgagaagggccctgagggcgcgtccggcaaggacggctctcgcggcctgacaggcccaatcggccctccaggccctgcgggcccaaacggcgagaagggcgagtccggcccttctggcccacctggcgcggcgggcacacgcggcgcgccaggcgaccgcggcgagaacggccctccaggccctgcgggcttcgcgggcccacctggcgcggacggccaaccaggcgcgaagggcgagcaaggcgagggcggccaaaagggcgacgcgggcgcgcctggcccacaaggcccttctggcgcgccaggccctcaaggcccaacaggcgtgtccggccctaagggcgcgcgcggcgcgcaaggcccacctggcgcgacaggcttcccaggcgcggcgggccgcgtgggccctccaggccctaacggcaacccaggcccttctggcccagcgggctccgcgggcaaggacggccctaagggcgtgcgcggcgacgcgggcccacctggccgcgcgggcgacgcgggctga

SEQ ID NO: 27 - DNA, 600 bp, synthetic construct
  source 1..600: mol_type = other DNA
ggtttgcaaggtatgccaggggaacggggtgcgtccgggatagccggggcaaaaggtgatcgaggcgatgtaggagaaaaaggcccagaaggggcgtcaggtaaggacggatctcgcggcttgacgggacctatcgggcctccaggtcccgccggccctaatggggaaaaaggcgagagtgggccgtctggtccgcccggcgccgctggcacacgtggagcgccgggcgatcgtggtgagaacggaccaccgggtcctgctggttttgcgggacctccgggagcagacggccagccgggcgctaaaggtgaacagggtgaaggtggccaaaaaggcgatgcaggcgcaccgggtccgcagggcccttcaggtgcaccgggtccacagggcccaactggcgtttcagggccgaaaggcgcaagaggtgctcagggtccgcccggggcaactgggtttcctggagcggccggccgtgttggacctccggggccgaacggaaaccctggaccgtctggaccagccggttcagcgggtaaggatggtcctaagggtgtaaggggtgacgcaggtccccctggacgtgcaggggatgcggggtag

SEQ ID NO: 28 - DNA, 600 bp, synthetic construct
  source 1..600: mol_type = other DNA
gggttacaaggtatgccgggagaacgtggagcgtcaggaattgctggggccaaaggtgatcgtggtgatgttggcgagaaagggcccgaaggcgcatctggtaaagatggctcacgcgggttaactggaccaatcggaccaccaggccccgctgggcctaatggtgaaaagggtgaaagtggcccttctggacccccaggagccgccggtacacgtggagcgccaggcgatcgtggcgaaaacggaccgcccggacctgcaggttttgcgggaccccctggagcagacggccaaccaggagcaaaaggtgagcaaggtgaaggtggacaaaagggagatgccggagcgccaggcccccaaggcccatcaggagctccaggacctcaaggtccaactggtgtatcagggcctaagggtgcgcgcggcgctcaaggaccgcctggcgcaactggctttccgggagctgctggtcgtgtgggcccgcctggcccaaacggaaatccaggcccttcaggcccggcgggctcagccggaaaagacggtccgaagggagtccgtggagatgcgggaccgccaggacgcgctggcgatgcaggctaa

SEQ ID NO: 29 - DNA, 600 bp, synthetic construct
  source 1..600: mol_type = other DNA
ggtttacagggaatgccaggggaacgcggcgcctcagggattgccggtgctaaaggagatcgtggcgacgtgggtgaaaagggtcccgagggagcatcaggtaaggatggttcccgtggtttgacgggacctattggacctccgggtcctgcaggtccgaacggcgaaaagggggaaagcgggcctagtggtccacccggcgccgcaggtacccgtggtgccccaggcgaccgcggggagaatggaccgcctggccctgccggttttgcgggtcctccaggagccgatgggcagcccggtgcaaaaggagagcagggagagggaggtcaaaagggagatgccggcgccccgggccctcagggaccaagcggtgcgccaggcccccagggtcctacgggtgttagcgggccgaaaggcgcacgcggagcgcagggcccacctggtgcaacaggcttcccaggagctgcggggcgcgtcggacctccgggacccaatggaaacccaggtccgtcagggccggcaggctccgcagggaaagatggtcccaaaggcgtgcgtggagacgcagggccccccggacgcgccggcgatgcgggataa

SEQ ID NO: 30 - DNA, 564 bp, synthetic construct; identical to SEQ ID NO: 3
  source 1..564: mol_type = other DNA

SEQ ID NO: 31 - protein, 957 aa, Gallus gallus
MAQLLRLFQTLLILLLRDYISAEDGETRASCRTAPADLVFILDGSYSVGPENFEIIKSWLVNITRNFDIGPKFIQVGVVQYSDYPVLEIPLGTHESTENLIKEMESIHYLGGNTKTGRAIQFAYDHLFAKSSRFLTKIAVVLTDGKSQDEVKDVAAEARKNKITLFAIGVGSEIEEDELKAIANKPSSTYVFYVEDYIAISRIKEVIKQKLCEESVCPTRIPVAARDEKGFDILVGLGVKKRVKKRIQIPTTNAKAYEVTSRVDLSELTRNVFPEGLPPSYVFVSTQRFKVKKTWDLWRVLSLDKRPQIAVTINGEEKTLSFTTTSLINGTQVITFAAPRVKTLFDEGWHQIRLLVTEDFVTLYIDDQEIETKPLHPVLGIYISGLTQIGKYSGKEETVQFDIQKLRIYCDPEQNNRETVCEIPGFNGECMNGPSDVGSTPAPCICPPGKQGPPGPKGDPGQPGNHGYPGQPGPDGKPGYQGSAGTPGIPGTPGVQGPRGLPGIKGEPGKDGTKGDRGLPGFPGLHGMPAPKGERGPKGDQGVPGIYGKKGSKGEKGDTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIRGINGQKGESGIQGLVGPPGVRGQPGDRGPPGPPGSDGKPAREFSEEFIRQVCSDVLRTQLPVILQSGRLQNCNHCQSQSASPGLPGPPGPRGPEGPRGFPGLPGNDGVPGLTGIPGRPGARGTRGLPGKNGAKGNQGIGVPGIQGPPGPPGPEGPPGMSKEGRPGERGQPGKDGDRGSPGMPGPVGPPGICDPSLCFSVIVGRDPFRKGPNY

SEQ ID NO: 32 - protein, 1420 aa, Acipenser schrenckii
MFSFVDSRTVLLLAAIQLCLLAVVKCQDVEVQQPGRKGQKGEPGDITDVVGPRGPGGPMGPPGEQGPRGERGDKGDKGGPGPRGRDGEPGTPGNPGPPGPPGPNGPPGLGGNFAAQMAGGFDEKAGGAQMGVMQGPMGPMGPRGPPGPTGAPGPQGFQGNPGEPGEPGAAGPLGPRGPPGPSGKPGEDGEAGKPGKSGERGSPGPQGARGFPGTPGLPGIKGHRGYPGLDGAKGEAGAAGSKGEAGSSGENGAPGPMGPRGLPGERGRNGPSGAAGARGNDGLPGPAGPPGPVGPAGAPGFPGSPGSKGEAGPTGARGPEGAQGPRGESGTPGSPGPSGASGNPGTDGIPGAKGSAGAPGIAGAPGFPGPRGPPGPQGATGPLGPKGQQGDPGIPGFKGEHGPKGEHGPAGPQGAPGPAGEEGKRGARGEPGAAGPLGPPGERGAPGNRGFPGQDGLAGPKGAPGERGQPGVGGPKGANGDPGRPGEPGLPGARGLTGRPGDAGPQGKGGPSGAAGEDGRPGPPGPQGARGQPGVMGFPGPKGANGEPGKAGEKGLVGPPGLRGLSGKDGETGAAGPPGPSGPAGERGEQGPPGPSGFQGLPGPPGPPGEGGKPGDQGVPGEAGAAGRAGPRGERGFPGERGSPGAQGLQGPRGLPGTPGTDGPKGATGPSGALGAQGPPGLQGMPGERGASGIAGAKGDRGDVGEKGPEGASGKDGSRGLTGPIGPPGPAGPNGEKGESGPSGPPGAAGTRGAPGDRGENGPPGPAGFAGPPGADGQPGAKGEQGEGGQKGDAGAPGPQGPSGAPGPQGPTGVSGPKGARGAQGPPGATGFPGAAGRVGPPGPNGNPGPSGPAGSAGKDGPKGVRGDAGPPGRAGDAGLQGAAGPPGEKGEPGEDGPPGPDGPSGPQGLGGNRGIVGLPGQRGERGFPGLPGPSGEPGKQGAPGGAGDRGPPGPVGPPGLSGPSGEPGREGNPGSDGPPGRDGSAGIKGDRGQTGPAGAPGAPGAPGSPGPVGPTGKQGDRGESGAQGPAGPSGPAGARGMAGPQGPRGDKGEAGETGERGQKGHRGFTGLQGLPGPPGTAGDQGAAGPAGPTGARGPPGPVGPHGKDGSNGQPGPIGPPGPRGRSGEVGPAGPPGNAGPPGPPGPPGPGIDMSAFAGLAAPEKAPDPMRYMRADEASSSLRQHDAEVDATLKSINNQIENIRSPEGSKKNPARTCRDLKLCHPDWKSGDYWIDPNQGCAVDAIKVFCNMESGETCVYPNPASIPRKNWWTSKSADCKHVWFGETMNGGFHFSYGDDSLAPNTASIQMTFLRLLSTEASQNLTYHCKNSIAYMDQSAGNLKKAVLLQGSNDVEIRAEGNSRFTYNVLEDGCTKHTDRWGKTVIEYKSQKTSRLPIVDIAPLDIGGSDQEFGVDIGPVCY

SEQ ID NO: 33 - protein, 213 aa, Gallus gallus
DTGFPGMPGRSGDPGRSGKDGLPGSPGFKGEVGQPGSPGLEGHRGEPGIPGIPGNQGAKGQKGEIGPPGLPGAKGSPGETGLMGPEGSFGLPGAPGPKGDKGEPGLQGKPGSSGAKGEPGGPGAPGEPGYPGIPGTQGIKGDKGSQGESGIQGRKGEKGRQGNPGLQGTEGLRGEQGEKGEKGDPGIRGINGQKGESGIQGLVGPPGVRGQPG

SEQ ID NO: 34 - protein, 199 aa, Acipenser schrenckii; identical to SEQ ID NO: 8

SEQ ID NO: 35 - protein, 189 aa, unidentified
GPQGVVGADGKDGTPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 36 - DNA, 570 bp, synthetic construct
  source 1..570: mol_type = other DNA
ggtccgcagggtgttgttggtgcagatggtaaagacggtaccccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 37 - protein, 187 aa, Homo sapiens
AGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVR

SEQ ID NO: 38 - DNA, 565 bp, synthetic construct
  source 1..565: mol_type = other DNA
tgcaggttttccgggtctgcctggtccggcaggcgaaccgggtcgtcatggtaaagatggtctgatgggtagtccgggttttaaaggtgaagcaggttcaccgggtgcacctggtcaggatggcacccgtggtgaaccgggtattccgggatttccgggtaatcgtggcctgatgggtcagaaaggtgaaattggtccgcctggtcagcagggtaaaaaaggcgcaccgggtatgccaggactgatgggttcaaatggcagtccgggtcagccaggcacaccgggttcaaaaggtagcaaaggcgaacctggtattcagggtatgcctggtgcaagcggtctgaaaggcgagccaggtgccaccggttctccgggtgaaccaggttatatgggtctgccaggtatccaaggcaaaaaaggtgataaaggtaatcagggcgaaaaaggcattcagggccagaaaggcgaaaatggccgtcagggtattccaggccagcagggcatccagggtcatcatggtgcaaaaggtgaacgtggtgaaaagggcgaaccaggtgttcgttaa

SEQ ID NO: 39 - protein, 429 aa, unidentified
GPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 40 - protein, 957 aa, Homo sapiens
MAHYITFLCMVLVLLLQNSVLAEDGEVRSSCRTAPTDLVFILDGSYSVGPENFEIVKKWLVNITKNFDIGPKFIQVGVVQYSDYPVLEIPLGSYDSGEHLTAAVESILYLGGNTKTGKAIQFALDYLFAKSSRFLTKIAVVLTDGKSQDDVKDAAQAARDSKITLFAIGVGSETEDAELRAIANKPSSTYVFYVEDYIAISKIREVMKQKLCEESVCPTRIPVAARDERGFDILLGLDVNKKVKKRIQLSPKKIKGYEVTSKVDLSELTSNVFPEGLPPSYVFVSTQRFKVKKIWDLWRILTIDGRPQIAVTLNGVDKILLFTTTSVINGSQVVTFANPQVKTLFDEGWHQIRLLVTEQDVTLYIDDQQIENKPLHPVLGILINGQTQIGKYSGKEETVQFDVQKLRIYCDPEQNNRETACEIPGFNGECLNGPSDVGSTPAPCICPPGKPGLQGPKGDPGLPGNPGYPGQPGQDGKPGYQGIAGTPGVPGSPGIQGARGLPGYKGEPGRDGDKGDRGLPGFPGLHGMPGSKGEMGAKGDKGSPGFYGKKGAKGEKGNAGFPGLPGPAGEPGRHGKDGLMGSPGFKGEAGSPGAPGQDGTRGEPGIPGFPGNRGLMGQKGEIGPPGQQGKKGAPGMPGLMGSNGSPGQPGTPGSKGSKGEPGIQGMPGASGLKGEPGATGSPGEPGYMGLPGIQGKKGDKGNQGEKGIQGQKGENGRQGIPGQQGIQGHHGAKGERGEKGEPGVRGAIGSKGESGVDGLMGPAGPKGQPGDPGPQGPPGLDGKPGREFSEQFIRQVCTDVIRAQLPVLLQSGRIRNCDHCLSQHGSPGIPGPPGPIGPEGPRGLPGLPGRDGVPGLVGVPGRPGVRGLKGLPGRNGEKGSQGFGYPGEQGPPGPPGPEGPPGISKEGPPGDPGLPGKDGDHGKPGIQGQPGPPGICDPSLCFSVIARRDPFRKGPNY

SEQ ID NO: 41 - protein, 10 aa, Gallus gallus
DTGFPGMPGR

SEQ ID NO: 42 - protein, 13 aa, Gallus gallus
DTGFPGMPGRSGD

SEQ ID NO: 43 - protein, 16 aa, Gallus gallus
DTGFPGMPGRSGDPGR

SEQ ID NO: 44 - protein, 19 aa, Gallus gallus
DTGFPGMPGRSGDPGRSGK

SEQ ID NO: 45 - protein, 29 aa, Gallus gallus
DTGFPGMPGRSGDPGRSGKDGLPGSPGFK

SEQ ID NO: 46 - protein, 9 aa, Gallus gallus
TGFPGMPGR

SEQ ID NO: 47 - protein, 17 aa, Gallus gallus
GFPGMPGRSGDPGRSGK

SEQ ID NO: 48 - protein, 15 aa, Gallus gallus
PGMPGRSGDPGRSGK

SEQ ID NO: 49 - protein, 12 aa, Gallus gallus
PGRSGDPGRSGK

SEQ ID NO: 50 - protein, 6 aa, Gallus gallus
SGDPGR

SEQ ID NO: 51 - protein, 19 aa, Gallus gallus
SGDPGRSGKDGLPGSPGFK

SEQ ID NO: 52 - protein, 10 aa, Gallus gallus
DGLPGSPGFK

SEQ ID NO: 53 - protein, 12 aa, Gallus gallus
DGLPGSPGFKGE

SEQ ID NO: 54 - protein, 25 aa, Gallus gallus
DGLPGSPGFKGEVGQPGSPGLEGHR

SEQ ID NO: 55 - protein, 40 aa, Gallus gallus
DGLPGSPGFKGEVGQPGSPGLEGHRGEPGIPGIPGNQGAK

SEQ ID NO: 56 - protein, 43 aa, Gallus gallus
DGLPGSPGFKGEVGQPGSPGLEGHRGEPGIPGIPGNQGAKGQK

SEQ ID NO: 57 - protein, 39 aa, Gallus gallus
GLPGSPGFKGEVGQPGSPGLEGHRGEPGIPGIPGNQGAK

SEQ ID NO: 58 - protein, 22 aa, Gallus gallus
PGSPGFKGEVGQPGSPGLEGHR

SEQ ID NO: 59 - protein, 15 aa, Gallus gallus
GEVGQPGSPGLEGHR

SEQ ID NO: 60 - protein, 30 aa, Gallus gallus
GEVGQPGSPGLEGHRGEPGIPGIPGNQGAK

SEQ ID NO: 61 - protein, 33 aa, Gallus gallus
GEVGQPGSPGLEGHRGEPGIPGIPGNQGAKGQK

SEQ ID NO: 62 - protein, 16 aa, Gallus gallus
RGEPGIPGIPGNQGAK

SEQ ID NO: 63 - protein, 15 aa, Gallus gallus
GEPGIPGIPGNQGAK

SEQ ID NO: 64 - protein, 18 aa, Gallus gallus
GEPGIPGIPGNQGAKGQK

SEQ ID NO: 65 - protein, 30 aa, Gallus gallus
GEPGIPGIPGNQGAKGQKGEIGPPGLPGAK

SEQ ID NO: 66 - protein, 54 aa, Gallus gallus
GEPGIPGIPGNQGAKGQKGEIGPPGLPGAKGSPGETGLMGPEGSFGLPGAPGPK

SEQ ID NO: 67 - protein, 10 aa, Gallus gallus
PGNQGAKGQK

SEQ ID NO: 68 - protein, 22 aa, Gallus gallus
PGNQGAKGQKGEIGPPGLPGAK

SEQ ID NO: 69 - protein, 15 aa, Gallus gallus
GQKGEIGPPGLPGAK

SEQ ID NO: 70 - protein, 13 aa, Gallus gallus
KGEIGPPGLPGAK

SEQ ID NO: 71 - protein, 12 aa, Gallus gallus
GEIGPPGLPGAK

SEQ ID NO: 72 - protein, 54 aa, Gallus gallus
GEIGPPGLPGAKGSPGETGLMGPEGSFGLPGAPGPKGDKGEPGLQGKPGSSGAK

SEQ ID NO: 73 - protein, 10 aa, Gallus gallus
IGPPGLPGAK

SEQ ID NO: 74 - protein, 24 aa, Gallus gallus
GSPGETGLMGPEGSFGLPGAPGPK

SEQ ID NO: 75 - protein, 24 aa, Gallus gallus
GSPGETGLMGPEGSFGLPGAPGPK

SEQ ID NO: 76 - protein, 26 aa, Gallus gallus
GSPGETGLMGPEGSFGLPGAPGPKGD

SEQ ID NO: 77 - protein, 27 aa, Gallus gallus
GSPGETGLMGPEGSFGLPGAPGPKGDK

SEQ ID NO: 78 - protein, 19 aa, Gallus gallus
TGLMGPEGSFGLPGAPGPK

SEQ ID NO: 79 - protein, 18 aa, Gallus gallus
GLMGPEGSFGLPGAPGPK

SEQ ID NO: 80 - protein, 11 aa, Gallus gallus
SFGLPGAPGPK

SEQ ID NO: 81 - protein, 18 aa, Gallus gallus
GDKGEPGLQGKPGSSGAK

SEQ ID NO: 82 - protein, 16 aa, Gallus gallus
KGEPGLQGKPGSSGAK

SEQ ID NO: 83 - protein, 15 aa, Gallus gallus
GEPGLQGKPGSSGAK

SEQ ID NO: 84 - protein, 42 aa, Gallus gallus
GEPGLQGKPGSSGAKGEPGGPGAPGEPGYPGIPGTQGIKGDK

SEQ ID NO: 85 - protein, 24 aa, Gallus gallus
GEPGGPGAPGEPGYPGIPGTQGIK

SEQ ID NO: 86 - protein, 27 aa, Gallus gallus
GEPGGPGAPGEPGYPGIPGTQGIKGDK

SEQ ID NO: 87 - protein, 30 aa, Gallus gallus
PGEPGYPGIPGTQGIKGDKGSQGESGIQGR

SEQ ID NO: 88 - protein, 27 aa, Gallus gallus
PGYPGIPGTQGIKGDKGSQGESGIQGR

SEQ ID NO: 89 - protein, 24 aa, Gallus gallus
PGIPGTQGIKGDKGSQGESGIQGR

SEQ ID NO: 90 - protein, 21 aa, Gallus gallus
PGTQGIKGDKGSQGESGIQGR

SEQ ID NO: 91 - protein, 22 aa, Gallus gallus
PGTQGIKGDKGSQGESGIQGRK

SEQ ID NO: 92 - protein, 14 aa, Gallus gallus
GDKGSQGESGIQGR

SEQ ID NO: 93 - protein, 15 aa, Gallus gallus
GDKGSQGESGIQGRK

SEQ ID NO: 94 - protein, 18 aa, Gallus gallus
GDKGSQGESGIQGRKGEK

SEQ ID NO: 95 - protein, 20 aa, Gallus gallus
GDKGSQGESGIQGRKGEKGR

SEQ ID NO: 96 - protein, 12 aa, Gallus gallus
KGSQGESGIQGR

SEQ ID NO: 97 - protein, 11 aa, Gallus gallus
GSQGESGIQGR

SEQ ID NO: 98 - protein, 12 aa, Gallus gallus
GSQGESGIQGRK

SEQ ID NO: 99 - protein, 15 aa, Gallus gallus
GSQGESGIQGRKGEK

SEQ ID NO: 100 - protein, 17 aa, Gallus gallus
GSQGESGIQGRKGEKGR

SEQ ID NO: 101 - protein, 19 aa, Gallus gallus
KGEKGRQGNPGLQGTEGLR

SEQ ID NO: 102 - protein, 25 aa, Gallus gallus
KGEKGRQGNPGLQGTEGLRGEQGEK

SEQ ID NO: 103 - protein, 18 aa, Gallus gallus
GEKGRQGNPGLQGTEGLR

SEQ ID NO: 104 - protein, 27 aa, Gallus gallus
GEKGRQGNPGLQGTEGLRGEQGEKGEK

SEQ ID NO: 105 - protein, 14 aa, Gallus gallus
GRQGNPGLQGTEGL

SEQ ID NO: 106 - protein, 15 aa, Gallus gallus
GRQGNPGLQGTEGLR

SEQ ID NO: 107 - protein, 21 aa, Gallus gallus
GRQGNPGLQGTEGLRGEQGEK

SEQ ID NO: 108 - protein, 24 aa, Gallus gallus
GRQGNPGLQGTEGLRGEQGEKGEK

SEQ ID NO: 109 - protein, 26 aa, Gallus gallus
GRQGNPGLQGTEGLRGEQGEKGEKGD

SEQ ID NO: 110 - protein, 13 aa, Gallus gallus
QGNPGLQGTEGLR

SEQ ID NO: 111 - protein, 19 aa, Gallus gallus
QGNPGLQGTEGLRGEQGEK

SEQ ID NO: 112 - protein, 22 aa, Gallus gallus
QGNPGLQGTEGLRGEQGEKGEK

SEQ ID NO: 113 - protein, 24 aa, Gallus gallus
QGNPGLQGTEGLRGEQGEKGEKGD

SEQ ID NO: 114 - protein, 16 aa, Gallus gallus
RGEQGEKGEKGDPGIR

SEQ ID NO: 115 - protein, 6 aa, Gallus gallus
GEQGEK

SEQ ID NO: 116 - protein, 15 aa, Gallus gallus
GEQGEKGEKGDPGIR

SEQ ID NO: 117 - protein, 9 aa, Gallus gallus
GEKGDPGIR

SEQ ID NO: 118 - protein, 6 aa, Gallus gallus
GDPGIR